(19) United States
(12) Patent Application Publication
(10) Pub. No.: US 2025/0157570 A1
(43) Pub. Date: May 15, 2025

(54) SYSTEMS AND METHODS FOR ANALYZING OMICS DATA

(71) Applicant: Seer, Inc., Redwood City, CA (US)

(72) Inventors: Theodore Platt, Danville, CA (US); Iman Mohtashemi, Mountain House, CA (US); Hugo Kitano, San Francisco, CA (US); Asim Siddiqui, San Francisco, CA (US); Amir ALAVI, San Francisco, CA (US); Harendra GUTURU, Oakland, CA (US)

(21) Appl. No.: 18/838,488

(22) PCT Filed: Feb. 15, 2023
(86) PCT No.: PCT/US2023/062684

§ 371 Date: Aug. 14, 2024

Related U.S. Application Data
(60) Provisional application No. 63/338,784, filed on May 5, 2022.
(60) Provisional application No. 63/310,516, filed on Feb. 15, 2022.

(51) Int. Cl. G16B 20/00; G16B 40/20; G16B 50/30

(52) U.S. Cl. CPC G16B 20/00; G16B 40/20; G16B 50/30

(57) ABSTRACT

In some aspects, the present disclosure provides a method for determining a polyamino acid descriptor associated with a biological state. The method can comprise removing technical variation from a proteomic dataset to generate a refined proteomic dataset, the technical variation arising from a predetermined non-biological factor, by training a neural network using a loss function. The loss function can be configured to increase a similarity between a first set of latent embeddings that are based on a first subset of polyamino acid descriptors in the proteomic dataset, wherein the first subset of polyamino acid descriptors are obtained from the same sample. The loss function can be configured to decrease the similarity between a second set of latent embeddings that are based on a second subset of polyamino acid descriptors in the proteomic dataset, wherein the second subset of polyamino acid descriptors are obtained from different samples.

### CROSS-REFERENCE

This application claims the benefit of U.S. Provisional Application No. 63/310,516, filed Feb. 15, 2022, and U.S. Provisional Application No. 63/338,784, filed May 5, 2022, each of which are incorporated herein by reference in their entirety.

### BACKGROUND

Biological samples contain a wide variety of proteins and nucleic acids. Computational methods are needed for elucidating the presence and concentration of proteins and nucleic acids as well as any correlations between proteins and nucleic acids that may be indicative of a biological state.

### SUMMARY

In some aspects, the present disclosure provides a method for determining a polyamino acid descriptor associated with a biological state, comprising: removing technical variation from a proteomic dataset to generate a refined proteomic dataset, the technical variation arising from a predetermined non-biological factor, by training a neural network to decrease a loss function configured to: increase a similarity between a first set of latent embeddings that are based on a first subset of polyamino acid descriptors in the proteomic dataset, wherein the first subset of polyamino acid descriptors are obtained from the same sample; and decrease the similarity between a second set of latent embeddings that are based on a second subset of polyamino acid descriptors in the proteomic dataset, wherein the second subset of polyamino acid descriptors are obtained from different samples; and identifying the polyamino acid descriptor that is associated with the biological state from the refined proteomic dataset.

In some embodiments, the proteomic dataset comprises a plurality of polyamino acid descriptors. In some embodiments, the plurality of polyamino acid descriptors comprises a plurality of polyamino acid intensities. In some embodiments, the plurality of polyamino acid intensities is based on a plurality of polyamino acid identifications, a plurality of surface types, or both. In some embodiments, the polyamino acid descriptor associated with the biological state comprises a polyamino acid identification. In some embodiments, the polyamino acid identification comprises a proteoform identification.

In some embodiments, the similarity is quantified using a similarity function comprising a distance-based similarity function, an angle-based similarity function, a set-based similarity function, or any combination thereof. In some embodiments, a local inverse Simpson's index (LISI) score of the refined proteomic dataset is less than 1.1, 1.2, 1.3, 1.4, 1.5, 1.6, 1.7, 1.8, 1.9, or 2 for a biological factor. In some embodiments, a local inverse Simpson's index (LISI) score of the refined proteomic dataset is greater than 1, 1.1, 1.2, 1.3, 1.4, 1.5, 1.6, 1.7, 1.8, 1.9, or 2 for a biological factor. In some embodiments, the biological factor comprises a biological sample type, a surface type, or both. In some embodiments, the surface type comprises a nanoparticle surface type. In some embodiments, a local inverse Simpson's index (LISI) score of the refined proteomic dataset is less than 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, or 4.0 for the predetermined non-biological factor. In some embodiments, a local inverse Simpson's index (LISI) score of the refined proteomic dataset is greater than 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, or 4.0 for the predetermined non-biological factor.

In some embodiments, the predetermined non-biological factor comprises using a different machine, using a different chromatography column, measuring at a different location, measuring at a different time, measuring by a different user, or any combination thereof. In some embodiments, the method further comprises receiving the plurality of polyamino acid descriptors measured from a plurality of mass spectrometers. In some embodiments, the method further comprises receiving the plurality of polyamino acid descriptors measured at different locations. In some embodiments, the method further comprises receiving the plurality of polyamino acid descriptors measured at different times. In some embodiments, the method further comprises receiving the plurality of polyamino acid descriptors measured by different users.

In some embodiments, the predetermined non-biological factor comprises collecting samples from a different location, collecting or processing samples by a different user, processing samples using different devices, transporting samples using a different condition, or any combination thereof. In some embodiments, the method further comprises receiving the plurality of polyamino acid descriptors measured from samples collected from different locations. In some embodiments, the method further comprises receiving the plurality of polyamino acid descriptors measured from samples collected or processed by different users. In some embodiments, the method further comprises receiving the plurality of polyamino acid descriptors measured from samples processed using different devices. In some embodiments, the method further comprises receiving the plurality of polyamino acid descriptors measured from samples transported under different conditions.

In some embodiments, the receiving is through the cloud. In some embodiments, the method further comprises: obtaining a plurality of mass spectrometry datasets obtained from a plurality of samples; normalizing, using a plurality of computing nodes, across the plurality of mass spectrometry datasets to generate a plurality of normalized mass spectrometry datasets, wherein the proteomic dataset comprises the plurality of normalized mass spectrometry datasets.

In some embodiments, the normalizing generates a set of aligned precursors for each mass spectrometry dataset in the plurality of mass spectrometry datasets. In some embodiments, the normalizing generates a set of relative abundances for each mass spectrometry dataset in the plurality of mass spectrometry datasets. In some embodiments, the normalizing comprises adjusting a set of polyamino acid intensities for each mass spectrometry dataset in the plurality of mass spectrometry datasets based on a plurality of feature values determined from the plurality of mass spectrometry datasets. In some embodiments, the normalizing comprises minimizing an objective function for a unique pair of mass spectrometry datasets in the plurality of mass spectrometry datasets for each computing node in the plurality of computing nodes. In some embodiments, the method further comprises generating a harmonized plurality of mass spectrometry datasets comprising a harmonized format based on the plurality of mass spectrometry datasets, wherein the harmonized format comprises (i) the plurality of mass spectrometry datasets in an indexed series and (ii) indices of the indexed series, such that the harmonized format is capable of being read in arbitrary slices in the indexed series and of inserting new datasets and/or being modified between arbitrary indices in the indexed series;

In some embodiments, the method further comprises: generating, based at least in part on a genomic dataset, a set of expressible proteoforms that can be expressed from a set of nucleic acids in the genomic dataset; and mapping the refined proteomic dataset to the set of expressible proteoforms, thereby determining a set of expressed proteoforms in the biological sample, wherein the polyamino acid descriptor is a proteoform in the set of expressed proteoforms.

In some aspects, the present disclosure provides a method of correcting batch effects in proteomic data, comprising: providing a neural network comprising: an input layer configured to receive at least one polyamino acid descriptor; a latent layer configured to output at least a latent descriptor, wherein the latent layer is operably connected to the input layer; and an output layer operably connected to the latent layer; wherein the latent layer and the output layer comprises one or more parameters; providing training data comprising a plurality of the at least one polyamino acid descriptors, wherein the plurality of polyamino acid descriptors comprises at least one value for a measured intensity of a given polyamino acid; training the neural network, by (i) inputting at least the plurality of polyamino acid descriptors at the input layer of the neural network, (ii) outputting a plurality of latent descriptors at the latent layer and a plurality of outputs at the output layer, and (iii) optimizing a loss function that is configured to guide the neural network towards learning a latent space comprising a plurality of embeddings for the plurality of polyamino acid descriptors by updating the one or more parameters, wherein the plurality of embeddings is invariant with respect to a predetermined non-biological factor.

In some embodiments, the method further comprises reconstructing, using a decoder neural network connected to the latent layer, a given plurality of polyamino acid descriptors based at least in part on a given plurality of embeddings to output a plurality of reconstructed polyamino acid descriptors, such that the plurality of reconstructed polyamino acid descriptors has a reduced variance with respect to the predetermined non-biological factor as compared to the plurality of polyamino acid descriptors. In some embodiments, the predetermined non-biological factor comprises at least one of: location of measurement, time of measurement, instrumentation component, or any combination thereof. In some embodiments, the instrumentation component comprises a mass spectrometry column. In some embodiments, the loss function comprises an adversarial triplet objective function comprising: L(a, p, n)=min Σᵢ₌₁^{N} max(d(aᵢ, p)−d(aᵢ, n)+α, 0), wherein a denotes a polyamino acid descriptor, wherein p denotes a positive reference for the polyamino acid descriptor, wherein n denotes a negative reference for the polyamino acid descriptor, and wherein a denotes a margin parameter.

In some embodiments, the loss function further comprises a classification loss function. In some embodiments, the classification loss function is configured to classify between distinct biological samples, distinct assay methods, or both. In some embodiments, the distinct assay methods comprises assays using distinct nanoparticles. In some embodiments, the loss function further comprises a reconstruction loss function. In some embodiments, the measured intensity comprises peptide intensity or protein group intensity. In some embodiments, the latent layer and the input layer are operably connected via one or more hidden layers. In some embodiments, the latent layer and the output layer are operably connected via one or more hidden layers.

In some aspects, the present disclosure provides a method of correcting batch effects in omic data, comprising: providing a neural network comprising: an input layer configured to receive at least one omic descriptor; a latent layer configured to output at least a latent descriptor, wherein the latent layer is operably connected to the input layer; and an output layer operably connected to the latent layer; wherein the latent layer and the output layer comprises one or more parameters; providing training data comprising a plurality of the at least one omic descriptors wherein the plurality of omic descriptors comprises at least one value for a measured intensity of a given omic signal; and training the neural network, by (i) inputting at least the plurality of omic descriptors at the input layer of the neural network, (ii) outputting a plurality of latent descriptors at the latent layer and a plurality of outputs at the output layer, and (iii) optimizing a loss function that is configured to guide the neural network towards learning a latent space comprising a plurality of embeddings for the plurality of omic descriptors by updating the one or more parameters, wherein the plurality of embeddings is invariant with respect to a predetermined non-biological factor.

In some aspects, the present disclosure provides a method for determining an omic descriptor associated with a biological state, comprising: removing technical variation from an omic dataset to generate a refined omic dataset, the technical variation arising from a predetermined non-biological factor, by training a neural network to decrease a loss function configured to: increase a similarity between a first set of latent embeddings that are based on a first subset of omic descriptors in the omic dataset, wherein the first subset of omic descriptors are obtained from the same sample; and decrease the similarity between a second set of latent embeddings that are based on a second subset of omic descriptors in the omic dataset, wherein the second subset of omic descriptors are obtained from different samples; and identifying the omic descriptor that is associated with the biological state from the refined omic dataset.

In some aspects, the present disclosure provides a computer-implemented method, implementing any one of the methods disclosed herein in a computer. In some aspects, the present disclosure provides a computer program product comprising a computer-readable medium having computer-executable code encoded therein, the computer-executable code adapted to be executed to implement any one of the methods disclosed herein. In some aspects, the present disclosure provides a non-transitory computer-readable storage media encoded with a computer program including instructions executable by one or more processors to implement any one of the methods disclosed herein. In some aspects, the present disclosure provides a computer-implemented system comprising: a digital processing device comprising: at least one processor, an operating system configured to perform executable instructions, a memory, and a computer program including instructions executable by the digital processing device to implement any one of the methods disclosed herein.

In some aspects, the present disclosure provides a method for identifying protein groups, comprising: obtaining a plurality of independently measured mass spectrometry data; subdividing each mass spectrometry data in the plurality of independently measured mass spectrometry data to provide a set of elements; distributing the set of elements onto a plurality of nodes; and generating, using the plurality of nodes, identifications of one or more biomolecules based at least in part on the set of elements.

In some embodiments, the obtaining comprises using an automated system to assay a plurality of biomolecules in one or more biological samples to produce the plurality of independently measured mass spectrometry data of the plurality of biomolecules.

In some embodiments, the automated system assays the plurality of biomolecules by (i) separating the plurality of biomolecules from the one or more biological samples using one or more surfaces and (ii) performing mass spectrometry on the plurality of biomolecules to produce the plurality of independently measured mass spectrometry data of the plurality of biomolecules.

In some embodiments, the separating comprises (i) contacting the one or more biological samples with the one or more surfaces to adsorb the plurality of biomolecules on the one or more surfaces and (ii) contacting the plurality of biomolecules on the one or more surfaces with a proteolytic enzyme to release the plurality of biomolecules from the one or more surfaces to produce an analyte for performing mass spectrometry, wherein the analyte comprises the released plurality of biomolecules.

In some embodiments, the one or more surfaces are disposed on one or more particles and the plurality of biomolecules comprises a plurality of proteins, such that the plurality of proteins form one or more protein coronas on the one or more particles when adsorbed on the one or more surfaces.

In some embodiments, the obtaining further comprises uploading the plurality of independently measured mass spectrometry data to a cloud-based computing system.

In some embodiments, the plurality of independently measured mass spectrometry data comprises mass spectrometry data obtained by performing mass spectrometry on a plurality of biological samples.

In some embodiments, the plurality of nodes comprises a distributed computing system.

In some embodiments, the set of elements comprise a set of mass spectrometry scans.

In some embodiments, a first node in the plurality of nodes is configured to transfer one or more annotations in a first mass spectrometry scan to a second node in the plurality of nodes.

In some embodiments, the identifications comprise one or more peptide spectral matches.

In some embodiments, the set of elements comprise a set of peptide identifications.

In some embodiments, a first node in the plurality of nodes is configured to transfer one or more probability values associated with a protein group assignment for one or more peptide identifications in the set of peptide identifications to a second node in the plurality of nodes.

In some embodiments, the identifications comprise one or more protein group identifications.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1C schematically illustrates a cloud scalable omics data analysis pipeline for processing MS datasets comprising a plurality of MS dataset filetypes, in accordance with some embodiments.

FIGS. 2A-2E schematically illustrate interfaces (i.e., an active programming interface (API), a graphical user interface (GUI), or both) for a cloud scalable omics data analysis pipeline, in accordance with some embodiments.

FIG. 3 shows a plot of total runtime as a function of the number of injections analyzed, in accordance with some embodiments.

FIG. 4 schematically illustrates a method for distributing a cached dataset and a task, in accordance with some embodiments.

FIG. 5 shows the computational costs for different processes in a label-free quantification analysis pipeline, in accordance with some embodiments.

FIGS. 6A-6B show the number of peptides identified using target-decoy and entrapment analysis, in accordance with some embodiments.

FIG. 7 schematically illustrates a process for performing alignment based on mass spectrometry datasets, in accordance with some embodiments.

FIG. 8 schematically illustrates a process for transmitting harmonized mass spectrometry datasets between computing nodes, in accordance with some embodiments.

FIG. 9 schematically illustrates a process for performing alignment based on harmonized mass spectrometry datasets, in accordance with some embodiments.

FIG. 10 schematically illustrates a computer system that is programmed or otherwise configured to implement methods provided herein.

FIG. 11 schematically illustrates a cloud-based distributed computing environment, in accordance with some embodiments.

FIG. 12 schematically illustrates a process for transmitting harmonized mass spectrometry datasets between computing nodes, in accordance with some embodiments.

FIG. 13A shows PCA embeddings of protein group log intensities of each run for four different covariates, in accordance with some embodiments. Qualitative and quantitative diagnostics using PCA reveal technical effects in the data. Each point is an LCMS run with its vector of protein group log intensities projected onto the first two principal components of the dataset. Points are colored by mass spectrometry machine, biosample, column, and nanoparticle. FIG. 13B shows principal components regression, showing that batch variables (LC column and MS instruments) add significant variance to the data over the analysis of the control plasma samples, in accordance with some embodiments. FIG. 13C shows Local Inverse Simpson's Index (LISI) score, which measures effective diversity of a label within small neighborhoods, which shows low levels of integration of batch variables, in accordance with some embodiments.

FIG. 14 shows dataset mixing and biological signal preservation of batch effect correction methods, in accordance with some embodiments. LISI scores are shown for various correction methods applied.

FIGS. 15A-15D shows quantitative and qualitative assessment of batch corrected representations for downstream tasks by comparing batch corrected representations for transfer learning, in accordance with some embodiments. FIG. 15A shows for MS Instruments (Machine), assessment of a KNN classifier trained on Orbitrap-1 and Orbitrap-2 data to classify amongst the two Biosamples with the test accuracy assessed on Orbitrap-3. This is repeated for testing on Orbitrap-1 and Orbitrap-2. FIG. 15B shows for MS Instruments (Machine), assessment of a KNN classifier trained on Orbitrap-1 and Orbitrap-2 data to classify amongst the three Nanoparticles with the test accuracy assessed on Orbitrap-3. This is repeated for testing on Orbitrap-1 and Orbitrap-2. Analogous procedure is repeated for Column. FIG. 15C shows PCA embeddings of the learned features for the DannClf model, in accordance with some embodiments. FIG. 15D shows embeddings of the learned features from the DannRecon model, in accordance with some embodiments.

FIGS. 16A-16B shows an adversarial neural network architecture for learning batch-invariant representations, in accordance with some embodiments. Protein group intensity data is fed forward through a fully connected ReLU encoder stage that is trained to perform poorly on a Triplet Loss which tries to discriminate technical batches. At the same time, this representation is trained to minimize either two classification tasks as in DannClf, FIG. 16A, and/or a reconstruction loss as in DannRecon, FIG. 16B.

### DETAILED DESCRIPTION

Though the human genome contains about 20,000 genes, some researchers estimate that the human proteome contains over 1 million proteins expressed from those genes. A number of different proteoforms can be expressed from a repertoire of various transcriptional, translational, and post-translational mechanisms (e.g., alternative splice forms, allelic variations, and protein modifications) that produce proteins that differ from those that comprise the canonical sequence expressed from the genes. Of the vast number of proteins estimated to exist in the human proteome, only a small fraction has thus been meaningfully identified and/or quantified in the human body.

Some of the challenges in identifying and quantifying the proteins is related to the rarity of certain proteins. For instance, human plasma contains protein species over a dynamic range that exceeds 12 magnitudes, where the top few proteins (e.g., albumin, transferrin, complement proteins, apolipoproteins, and alpha-2-macroglobulin) comprise 95% of the mass of protein in the plasma, and most of the protein species comprise the remaining 5%. Some of the protein species exist in the nanograms per milliliter ranges (e.g., transforming growth factor beta-1-induced transcript 1 protein at ˜10 ng/ml; fructose-bisphosphate aldolase A at ˜20 ng/ml; thioredoxin at ˜18 ng/ml; and L-selectin at ˜92 ng/ml), and some proteins are expected to be present at levels even beneath that range. Liquid chromatography coupled with mass spectrometry (LC-MS) or tandem mass spectrometry (LC-MS/MS) have grown into ubiquitous detection platforms due to their speed, sensitivity, and breadth of applications. LC-MS and LC-MS/MS can be used to identify protein species, however, due to the stochastic nature of the methods, only a fraction of ionic species that are generated at a time from a given sample may be selected for acquiring mass spectra. As a result, the presence of species that are highly abundant compared to the rare species can create an overwhelming amount of signals that make the rare species elusive.

Some aspects of the PROTEOGRAPH™ technology aims to solve some of these challenges by “compressing” the dynamic range of protein species in a sample. Some aspects of the PROTEOGRAPH™ technology operates based on non-specific binding of proteins to nanoparticle surfaces to form protein coronas. Without requiring a presence of a specific entity that is configured for binding to a singular specific protein (e.g., as in immunoassays), the non-specific binding can result in a dynamic range compression of proteins bound to the nanoparticle surfaces while capturing a wide variety of proteins. In other words, the relative abundance of proteins in the sample can be modified on the nanoparticle surfaces, such that the rare proteins are relatively more abundant, and the highly abundant proteins are relatively less abundant compared to the original sample. The proteins can then be separated from the sample and analyzed, for example, with mass spectrometry. The compressed dynamic range can allow rare proteins to comprise a higher fraction of ionic species, thereby allowing higher probability for detecting those rare proteins in a MS experiment. Though the above example is described in terms of proteins, other biomolecule classes (e.g., lipids, sugars, etc.) can be similarly targeted. Other aspects of the PROTEOGRAPH® technology include controlled automation of the PROTEOGRAPH™ workflow that increases speed/throughput and accuracy/reliability.

While the introduction of the PROTEOGRAPH™ technology increased the number of proteins that can be detected from samples, another challenge is presented, which is to find biomarkers and/or therapeutic targets among those proteins. As the number of proteins that can be considered for diagnostic or therapeutic potential increases, the sample size may also be increased in order to effectively screen for the relevant proteins. Due to individual differences in biology between humans, thousands of proteins can have varying levels in plasma samples between two individuals. Therefore, samples from hundreds or thousands of individuals may be experimented with to identify meaningful and systematic signals that have clinical relevance.

Currently available platforms, software, and data structures used for processing mass spectrometry dataset have numerous limitations that make it difficult to process hundreds and thousands of samples. When conducting large-scale cohort studies, technical confounding can be introduced as samples are acquired, processed, and analyzed across by different users, different machines, different locations, different times, and etc. For instance, technical confounding can be introduced when samples are analyzed using different MS instruments, LC columns, dates, and geographic locations. In order to integrate these samples across datasets for joint analyses, one may diagnose such batch effects and apply methods to correct them.

Some batch correction methods used in proteomics, transcriptomics, and other omics are non-parametric to reduce assumptions made on the data. Some examples are methods based on simple median normalization as in MSSTATS™; nearest neighbor matching like MNN™ and SCANORAMA™; and HARMONY™ which is aniterative clustering and vector translating algorithm. Parametric approaches include COMBAT™ which is based on empirical Bayes, and deep-learning based approaches such as SCVI™.

In some aspects, the present disclosure provides a method of using domain transfer, or domain adaptation. Domain adaptation can be applied to train a machine learning algorithm under a source domain, and then tasked with predicting in a target domain. The data in each case may come from different underlying distributions (domain shift). In some aspects, the present disclosure provides a method for characterizing and/or correcting batch effects in proteomics data. In some embodiments, a batch effect comprises technical variation. In some embodiments, a batch effect does not comprise biological relevant variation. In some embodiments, the method uses domain adaptation. Supervised adversarial neural network can be trained to learn batch-invariant representations of proteomics data. The method can remove at least a portion of the technical variation, which can lead to at least 20% improvement in dataset homogenization. Meanwhile, variation in the data due to clinically relevant biological differences can be preserved.

Using the method of the present disclosure, proteomic data from a large number of data sources can be better integrated to provide more accurate and reliable biological insights. There are a number of benefits of reducing data variation arising from factors which do not carry biological relevance (e.g., variation arising from the specific user that ran the experiment, the specific machine or instrumentation used to take the measurement, and sporadic differences in ambient conditions). First, larger studies can be carried out. Harmonizing data across different platforms, users, laboraties, and etc., can allow screening a larger number of proteomic signatures by leveraging the attainment of and analyses of proteomic data at scale. Second, amount of data required to detect a relevant signal, may be reduced. Sporadic and unimportant variations in data, when filtered out, can increase the visibility of biologically relevant signals and improve the confidence of detecting biologically relevant signals.

Another aspect of the present disclosure provides cloud scalable omics data analysis pipeline using serverless task infrastructure (i.e., introducing cloud scalable multi-omics pipelines using AWS Step functions and serverless task infrastructure), for instance, as disclosed in PCT/US2022/037003 which is incorporated by reference in its entirety herein. Some bioinformatic platforms use closed-source software and data structures, which make it difficult to cooperatively leverage mass spectrometry datasets across different users. For instance, some LC-MS and LC-MS/MS bioinformatic algorithms and software are built for desktop environments which are not easily leveraged for high-performance applications. Some LC-MS bioinformatic algorithms are closed-source “black-box” executables and cannot be distributed natively. Closed-source software can be difficult to leverage in distributed computing environments including cloud-based environments. Some software supporting a LC-MS instrument may output file formats that are different from another software supporting the LC-MS instrument. Dissonance between file formats obtained from different software or different mass spectrometry instruments can pose challenges in integrating data at scale. In some cases, differential proteomics data analysis of large datasets (‘group runs’) may require data aggregation (e.g., during chromatographic alignment or Protein Inference) of numerous and large datasets, which can be memory/disk limited in some environments, some existing applications are not designed for increasing compute and memory demands, and some software supporting a LC-MS instrument may not be designed optimally for computational speed or for efficiency in memory usage.

Improved computational platforms of the present disclosure can advantageously provide an ability to analyze mass spectrometry datasets from hundreds, thousands, or more mass spectrometry experiments. Some of the challenges addressed by the systems and methods of the present disclosure include harmonizing a large variety of mass spectrometry dataset formats so that the datasets can be processed together. Another aspect includes providing a number of mass spectrometry analysis algorithms on a singular platform. The harmonization employed by the computational platforms of the present disclosure can allow users of the platform to utilize mass spectrometry datasets from disparate sources (e.g., datasets from different machines, different locations, different times, etc.) using a variety of mass spectrometry analysis algorithms (some current algorithms may require a specific type of a dataset format—by harmonizing the datasets, algorithms can be used a harmonized dataset regardless of the source). The modularization can allow users of the platform to write new programs and computational protocols for processing or analyzing mass spectrometry datasets using the variety of mass spectrometry analysis algorithm. The computational platforms of the present disclosure can provide remote access to multiple users and entities over a network. Datasets can be shared between remote users in real-time in harmonized formats, regardless of the format that the datasets were originally generated by the users. The following paragraphs provide illustrative embodiments that detail various aspects of the computational platforms of the present disclosure.

Another aspect of the present disclosure provides methods and systems for performing fast, scalable, deep, and unbiased plasma proteomics. In some cases, the methods and systems may be used to identify known and/or novel biomarkers for diseases. In some cases, the methods and systems may be used to facilitate identification of disease-relevant protein variants, for instance, as disclosed in PCT/US2023/060271, which is incorporated by reference in its entirety herein. Important advances in characterizing the proteomic landscape of lung cancers such as non-small cell lung cancer (NSCLC) and squamous cell lung cancer have identified important protein biomarkers. However, relatively few proteoforms relevant to lung cancer have been identified. Readout technologies such as high resolution quantitative mass spectrometry (MS) can be employed to infer and to quantify peptides and proteins with high confidence (e.g., <1% false discovery rate (FDR)). However, large-scale LC-MS/MS-based proteomics studies can be challenging due to lengthy workflows required to achieve deep (e.g., broad detection of proteins across the dynamic range, from high to low abundance proteins) and unbiased (e.g., hypothesis-free detection) sampling of clinically relevant biospecimens with large dynamic ranges of protein abundances, such as blood plasma. While LC-MS and LC-MS/MS methodologies may offer the capability to infer proteoforms, peptide identification in LC-MS/MS-based proteomic data may rely on protein databases, such as UniProt, which may exclude proteoforms that may be present in an individual's proteome. In some cases, the methods and systems may be used to observe examples of alternative exon usage. In some cases, the methods and systems may be used to identify proteoforms arising from alternative splicing. In some cases, the methods and systems may be used to identify proteoforms arising from genetic variation. In some cases, the methods and systems may be used to identify proteoforms based at least partially on custom protein databases generated from subject-matched genotype data, such as whole exome sequencing (WES) data. In some cases, the methods and systems may be used to discover new proteoforms. In some cases, the methods and systems may be used to identify proteoforms that would otherwise not be identified using protein affinity-based targeted technologies. In some cases, the methods and systems disclosed herein may be used to support enhanced understanding of human health and disease by identifying proteoforms.

### Method for Learning Batch-Invariant Representations

In some aspects, the present disclosure provides a method for determining a polyamino acid descriptor associated with a biological state. In some embodiments, the method comprises removing technical variation from a proteomic dataset to generate a refined proteomic dataset. The technical variation can arise from a predetermined non-biological factor. Removing can be performed by training a neural network. The neural network can be trained to reduce a loss function configured to increase a similarity between a first set of latent embeddings that are based on a first subset of polyamino acid descriptors in the proteomic dataset. The first subset of polyamino acid descriptors can be obtained from the same sample. The neural network can be trained to reduce a loss function configured to decrease the similarity between a second set of latent embeddings that are based on a second subset of polyamino acid descriptors in the proteomic dataset. The second subset of polyamino acid descriptors can be obtained from different samples. The method can comprise identifying the polyamino acid descriptor that is associated with the biological state from the refined proteomic dataset.

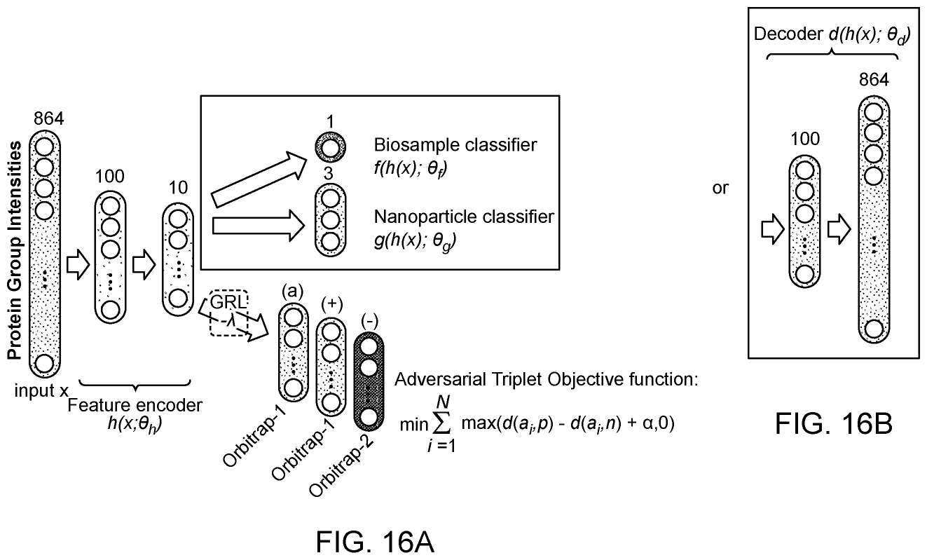

FIG. 16 shows an adversarial neural network architecture for learning batch-invariant representations, in accordance with some embodiments. The neural network can receive input data, x, at an input layer. The input data can be processed with one or more neural network layers (e.g., comprised in a feature encoder; h(x,θₕ)) to generate a latent embedding of the input data. The latent embedding can be further processed with a gradient reversal layer (“GRL”) which can act as an identity function during forward propagation, and can change the sign of the gradient during back propagation.

The neural network can be trained to optimize a loss function such that variance in the input data arising from non-biological factors is at least partially removed. In some embodiments, this can be performed by using the following loss function with the gradient reversal layer:

$$L ( a , p , n ) = min {\sum }_{i = 1}^{N} max ( d ( {a}_{i} , p ) - d ( {a}_{i} , n ) + α , 0 )$$

wherein L denotes the loss function, wherein a denotes a polyamino acid descriptor, wherein p denotes a positive reference for the polyamino acid descriptor, wherein n denotes a negative reference for the polyamino acid descriptor, wherein N denotes the number of polyamino acid descriptors to iterate over, wherein d denotes a distance function, and wherein a denotes a margin parameter.

In this example, d(aᵢ, n) can express a first objective for optimization, which can be the distance between an input polyamino acid descriptor selected from the training data and a negative reference. The negative reference can be from a different batch as the selected input, but obtained using the same biological sample, optionally using the same nanoparticle surface for biomolecule enrichment. To remove technical variation, this first objective can be reduced, such that the latent embeddings of two polyamino acid descriptors that arise from measurements of the same biological sample, optionally using the same nanoparticle surface, will be more similar (e.g., closer) in the latent space. For example, measurements of a standard plasma sample across different batches, may be embedded into the latent space to discard certain technical variation that arise from non-biological factors. The measurements of the standard plasma sample, can in theory, map to the same coordinate in the latent space, or at least be close to one another in the latent space.

Meanwhile, d(aᵢ, p) can express a second objective for optimization, which is the distance between a selected input polyamino acid descriptor and a positive reference. The positive reference can be from the same batch as the selected input, but obtained using a different biological sample, optionally using a different nanoparticle surface for biomolecule enrichment. To remove technical variation, this second objective can be increased, such that the latent embeddings of two polyamino acid descriptors that arise from measurements of different biological samples, optionally using different nanoparticle surfaces, will be more different (e.g., distant) in the latent space. For example, measurements of plasma samples, the plasma samples which are known to have clinically relevant differences, may be embedded into the latent space to preserve relevant variation that arise from biological (e.g., clinically-relevant) factors. The measurements of the plasma samples, can in theory, map to distant coordinates in the latent space.

During training, the neural network can be guided to update its parameters towards achieving the first, the second, or both objectives. In the example, the gradient reversal layer is used in the neural network to optimize a loss function, that is in effect:

$$L ( a , p , n ) = min [ - 1 * {\sum }_{i = 1}^{N} max ( d ( {a}_{i} , p ) - d ( {a}_{i} , n ) + α , 0 ) ]$$

Thus, the feature encoder of the neural network can update its parameters to embed polyamino acid descriptors that arise from measurements of different biological samples, optionally using different nanoparticle surfaces, to be more different; and polyamino acid descriptors that arise from measurements of the same biological sample, optionally using the same nanoparticle surface, to be more similar in the latent space.

Thus, the neural network can be used to process an input dataset of polyamino acid descriptors from different batches to output a refined dataset. The different batches may be measured from different machines (e.g, having different chromatography columns, different mass spectrometers, or different models of the PROTEOGRAPH™ machine), at different dates or times, different ambient conditions (e.g., ambient temperature, pressure, or humidity), by different users of a machine, different batches of surfaces for biomolecule enrichment (e.g., PROTEOGRAPH™ nanoparticles), or any combination thereof. The different batches may also include samples collected from different sites (e.g., blood collection sites), samples collected or processed by different people (e.g., different phlebotomists or lab technicians), samples processed using different devices (e.g., different centrifuges for plasma collection), different shipping conditions, or any combination thereof. The refined dataset can comprise reduced technical variation, e.g., arising from non-biological factors. The refined dataset can preserve biologically-relevant variation from the input dataset.

In some embodiments, the neural network can be trained using a classifer. As shown in FIG. 16, the classifer can be configured to output a vector that classifies whether an input polyamino descriptor was measured from a certain type of biological sample. In some embodiments, the classifer can be configured to output a vector that classifies whether an input polyamino descriptor was measured using a certain nanoparticle. The classifier can be a neural network that receives the latent embedding, from the feature encoder, as input in order to generate the output vector. The classifer can be trained in conjunction with the feature encoder, such that the feature encoder learns to preserve information relevant to the classification task. In this example, the preserved information can be the type of biological sample, the nanoparticle used, or both.

In some embodiments, the neural network can be trained using a feature decoder neural network. As shown in FIG. 16, the feature decoder (d(h(x);θ_{d})) can receive the latent embedding, from the feature encoder, as input to generate an output vector of the same shape and size as the input polyamino descriptor. The feature decoder can be trained in conjunction with the feature encoder, such that the feature encoder is encouraged to learn to preserve variance in the input dataset as much as possible.

While the above example has been described using polyamino acid descriptors, those skilled in the art will recognize that the neural network can be used to process an input dataset comprising other omic data. Omic data can comprise proteomic data, genomic data, transcriptomic data, or any combination thereof. Omic data can be obtained using next-generation sequencing, proximity-ligands, immunoassays, etc.

Those skilled in art will recognize that various values of α, the margin parameter, can be used. In some embodiments, the margin parameter can be at least 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, or 10 times the norm of the input dataset. In some embodiments, the margin parameter can be at most 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, or 10 times the norm of the input dataset. Similarly, those skilled in art will recognize that various alternative values can be used for the second operand in the max function, instead of 0.

Those skilled in the art will recognize that various functions may be used in place of the distance function to achieve the same or similar effects of removing technical variation while preserving biological-relevant variation in the input data. In some embodiments, a similarity function can be used. In some embodiments, the similarity function comprises a distance-based similarity function, an angle-based similarity function, a set-based similarity function, or any combination thereof. In some embodiments, the angle-based similarity function is a cosine similarity function. In some embodiments, the distance-based similarity function may be based at least in part on a Euclidean distance. In some embodiments, the set-based similarity function may be a clustering function. Those skilled in the art will recognize that the precise form of the similarity function can selected or varied based on the support for the latent space, for example, the latent space can be in a Euclidean coordinate system, cylindrical coordinate system, spherical coordinate system, among other systems.

### Storing and Processing Mass Spectrometry Datasets

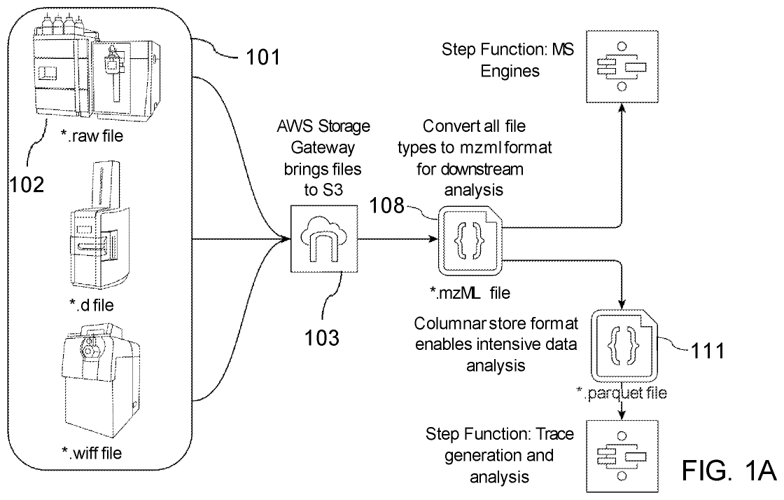

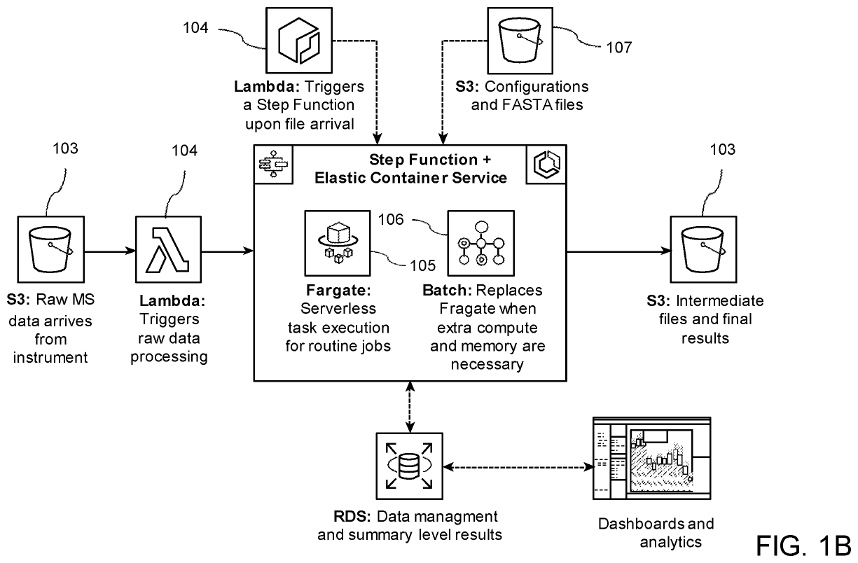

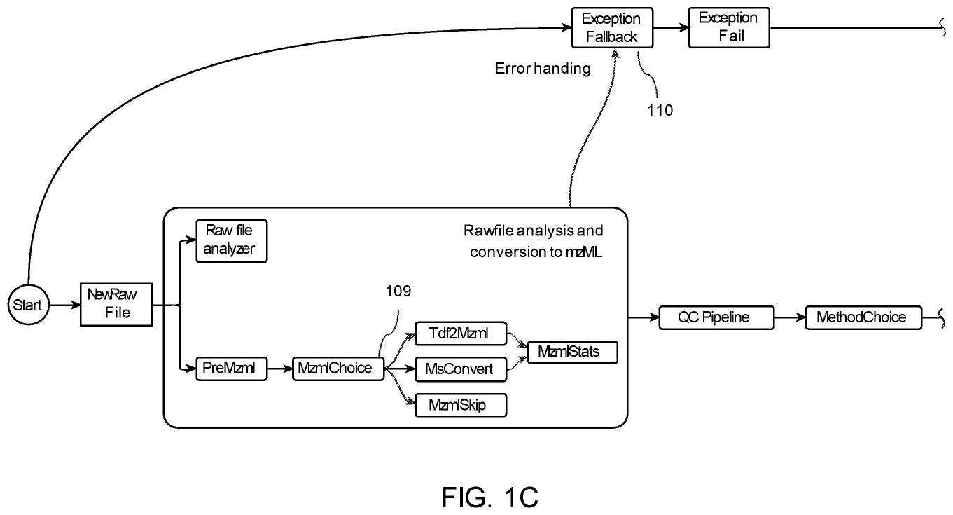

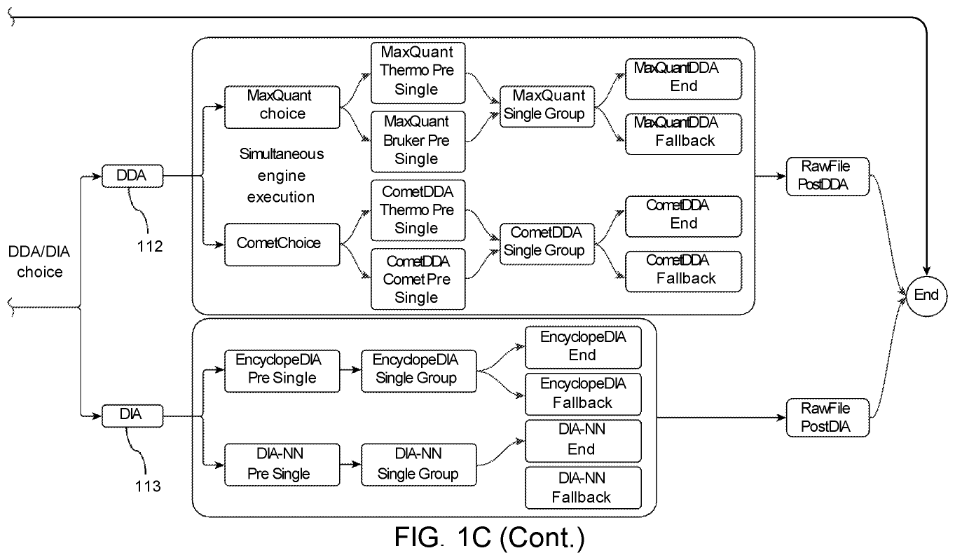

In some aspects, the present disclosure provides a computer-implemented method for storing and processing mass spectrometry datasets on a cloud platform. FIGS. 1A-1B schematically illustrate a cloud scalable mass spectrometry data analysis pipeline for processing outputs from a plurality of mass spectrometry (MS) instrument types, in accordance with some embodiments. The computer-implemented method can comprise transmitting a mass spectrometry dataset (101) to a computer system. The transmitting can be performed autonomously. The computer-implemented method can comprise receiving the mass spectrometry dataset at the computer system. The computer-implemented method can comprise transmitting a plurality of mass spectrometry datasets to the computer system. The computer-implemented method can comprise receiving the plurality of mass spectrometry datasets at the computer system.

The mass spectrometry dataset can be generated by a mass spectrometer (102). The mass spectrometry dataset can be generated by a plurality of mass spectrometers. The mass spectrometer can transmit the mass spectrometry dataset autonomously. The mass spectrometry dataset can comprise data from a set of experiments, a set of measurements (e.g., data from one or more injections in a tandem liquid chromatography-mass spectrometry experiment) in a single experiment, or both. The mass spectrometry dataset can be accompanied by a user-specified recipes or settings for processing the mass spectrometry dataset. The plurality of mass spectrometers can be at different locations. The plurality of mass spectrometers can generate the mass spectrometry datasets during the same time period or at different time periods from one another. The plurality of mass spectrometers may be operated by the same entity or different entities (e.g., customers, users, companies, labs, researchers, etc.). The mass spectrometer can comprise a plurality of mass spectrometer types or commercial models. The plurality of mass spectrometer types or commercial models can generate a plurality mass spectrometry datasets comprising a variety of data formats. The mass spectrometry dataset can comprise one of a plurality of mass spectrometry dataset formats. Mass spectrometry dataset formats can include *.raw format, *.d format, *.wiff format, *.txt format, or any other format used for storing or processing mass spectrometry data. The mass spectrometry dataset can be stored on a cloud-based storage system (103).

Upon receiving the mass spectrometry dataset, an event signal can be generated by the computer system. The event signal can be configured to trigger an event on the computer system. The event signal can be used as a trigger to create a serverless cloud computing instance for running a data processing routine. The event signal can be used as a trigger to create a container for running a data processing routine. The event signal can be used to trigger (104) the data processing routine to be performed on the mass spectrometry dataset using the serverless cloud computing instance (105). If the a serverless cloud computing instance cannot be instantiated (e.g., when resources for serverless cloud computing are limited), the data processing routine can be performed using a server cloud computing instance (106). The size of computational resources of the serverless cloud computing instance can be based on the mass spectrometry dataset. For instance, the size of the computational resources can be scaled autonomously based on the size and/or complexity of the mass spectrometry dataset. A computational resource can comprise memory, storage, number of processors, or any combination thereof. The computer-implemented method can comprise receiving a second mass spectrometry dataset. A second event signal can be generated based on the second mass spectrometry dataset. A second serverless cloud computing instance can be created based on the second event signal. A second data processing routine can be performed based on the second mass spectrometry dataset using the second serverless cloud computing instance. The data processing routine and the second data processing routine can be performed in parallel. In some embodiments, the computer-implemented method can process and/or store genomic datasets (107) on the cloud platform. For each new mass spectrometry dataset that is received, a new serverless cloud computing instance can be instantiated to perform the data processing routine on each mass spectrometry dataset.

The data processing routine can comprise generating a harmonized mass spectrometry dataset (108) comprising a harmonized data format based on the mass spectrometry dataset. A harmonized mass spectrometry dataset can refer to a mass spectrometry dataset that has a been transformed to have a consistent format with another mass spectrometry dataset. The harmonized mass spectrometry dataset can be an *.xml, *.h5, *.mzml, *.parquet, or any appropriate format. The harmonized mass spectrometry dataset can comprise headers, sections, indices, columns, rows, graphs and any other organizational structure for organizing MS data. An example of a data processing routine is schematically illustrated in FIG. 1C. The data processing routine can receive a MS dataset. Depending on the format of the MS dataset, different conversion algorithms (109) can be used to generate the harmonized MS dataset. The data processing routine can comprise error and/or exception handling routines (110). The error and/or exception handling routines can notify an entity (e.g., a user) of an error. The error and/or exception handling routines can provide suggestions for troubleshooting or solving the error. The data processing routine can comprise generating a plurality of harmonized mass spectrometry datasets comprising the harmonized data format based on a plurality of mass spectrometry datasets. In some embodiments, the harmonized mass spectrometry dataset comprises a columnar format (111), e.g., *.parquet format. The data processing routine can comprise storing the harmonized mass spectrometry dataset on storage system. The storage system can be an object-based storage system. The object-based storage system can be partitioned to create space for storing the harmonized mass spectrometry dataset. The space can be autonomously scaled based on the size of the harmonized mass spectrometry dataset. The data processing routine can comprise processing the harmonized mass spectrometry dataset after retrieving it from the storage system.

The data processing routine can comprise performing a polyamino acid search to generate a plurality of polyamino acid identifications. Polyamino acid can refer to a peptide, a protein, or any molecule or complex comprising two or more amino acids in a sequence. A polyamino acid search can refer to a process for determining an identity (e.g., a sequence, a protein group, an isoform in a protein group, etc.) of a polyamino acid based on information about the polyamino acid. The data processing routine can comprise performing a plurality of polyamino acid searches. The polyamino acid search can be based on the harmonized mass spectrometry dataset and a data acquisition mode of the mass spectrometry dataset. The data acquisition mode of the mass spectrometry dataset can be data dependent acquisition (DDA) or data independent acquisition (DIA). The polyamino acid search can be one or more of a plurality of search modes. The plurality of search modes can comprise a plurality of DDA search modes (112) or a plurality of DIA (113) search modes. For instance, a DDA search mode can be MaxQuant, CometDDA, or another search mode configured to process DDA datasets. A DIA search mode can be EncylopeDIA, DIA-NN, or another search mode configured to process DIA datasets. The data processing routine can comprise storing the plurality of polyamino acid identifications on the storage system. The storage system can be an object-based storage system. The storage system can be a distributed relational storage system. The storage system can be a non-relational storage system. The storage system can be a public storage system, a shared storage system between two or more entities, or a private storage system.

The data processing routine can comprise performing protein grouping based on the plurality of polyamino acid identifications to generate a plurality of protein groups. Performing the protein grouping can comprise subdividing the harmonized mass spectrometry dataset to generate a plurality of mass spectrometry scans. Performing the protein grouping can comprise distributing the plurality of mass spectrometry scans onto a plurality of computing nodes. Performing the protein grouping can comprise performing the plurality of polyamino acid searches, using the plurality of computing nodes, to generate the plurality of protein groups. The data processing routine can comprise normalizing the mass spectrometry dataset. The data processing routine can comprise alignment, quantification, or both.

In some embodiments, the computer-implemented method comprises processing a mass spectrometry (MS) dataset to store a trace in a distributed storage system. The computer-implemented method can comprise extracting a plurality of signals from the MS dataset. Each signal in the plurality of signals can comprise a mass-to-charge ratio (m/z), a retention time, and an intensity. The plurality of signals can be extracted when the m/z of a signal in the MS dataset is within a predetermined range from a reference m/z of a reference feature in the MS dataset. The trace comprising the plurality of signals in association with an identifier for the reference feature can be stored in the distributed storage system. The trace can be loaded into a cache memory for further processing, for example, visualizing the trace, determining a quality of the trace, quantifying the statistics of the trace, and etc.

In some aspects, the present disclosure provides a computer-implemented system for storing mass spectrometry datasets on a cloud platform. The computer-implemented system can comprise at least one digital processing device. The at least one digital processing device can comprise at least one processor, an operating system configured to perform executable instructions, a memory, and a computer program including instructions executable by the digital processing device. The instructions can comprise a first instruction configured to generate an event signal when a mass spectrometry dataset is received by the computer-implemented system. The mass spectrometry dataset can comprise at least one of a plurality of formats. The instructions can comprise a second instruction configured to be triggered by the event signal to instantiate a serverless cloud computing instance. The instructions can comprise a third instruction configured to perform a data processing routine using the serverless cloud computing instance. The data processing routine can comprise generating a harmonized mass spectrometry dataset comprising a harmonized data format based on the mass spectrometry dataset. The data processing routine can comprise storing the harmonized mass spectrometry dataset on an object-based storage system.

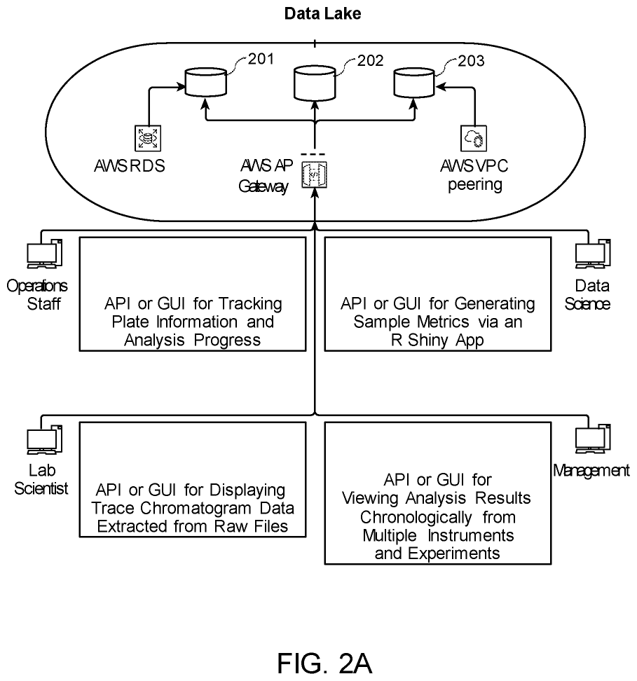

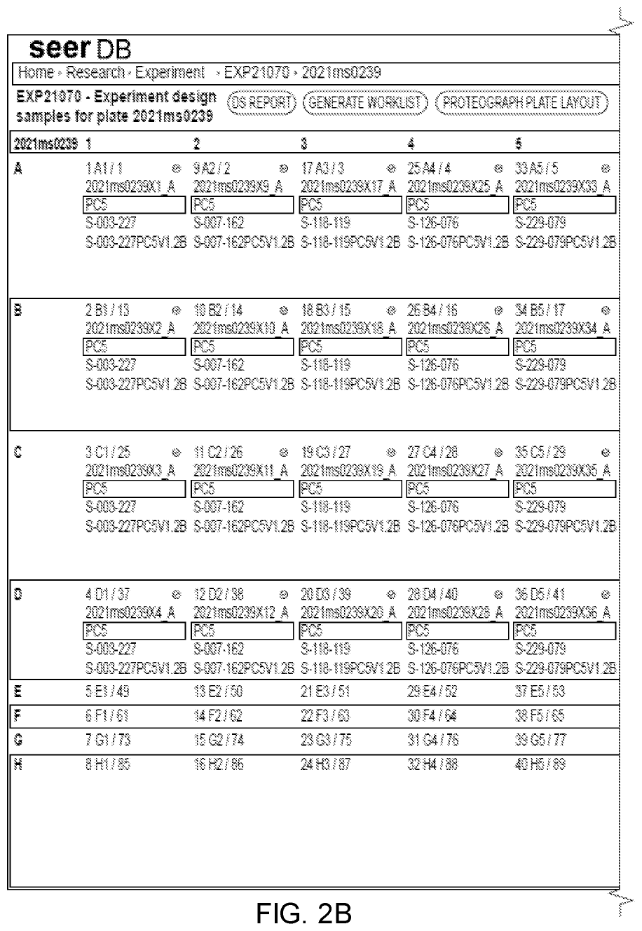

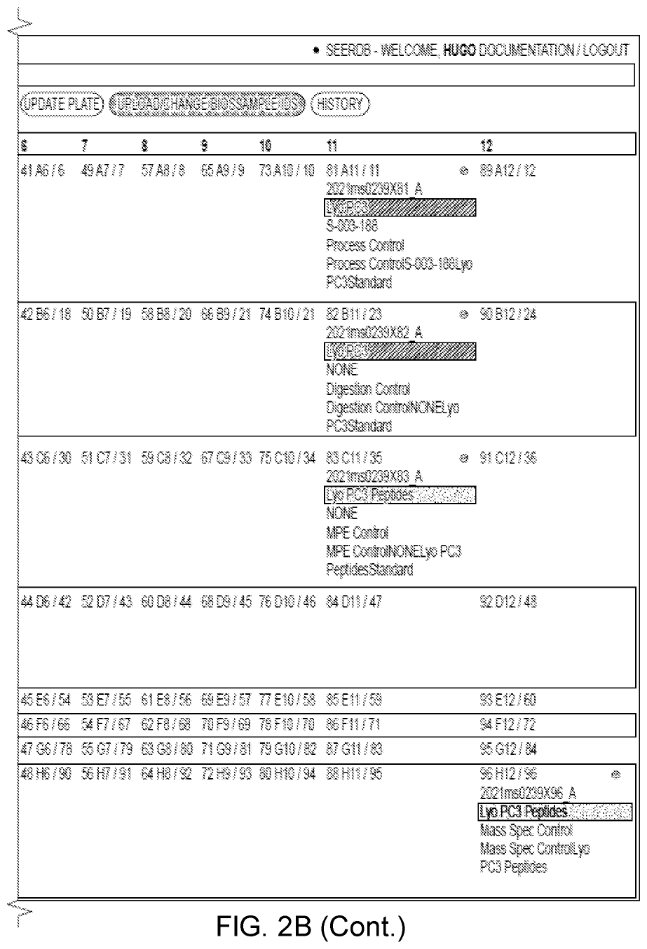

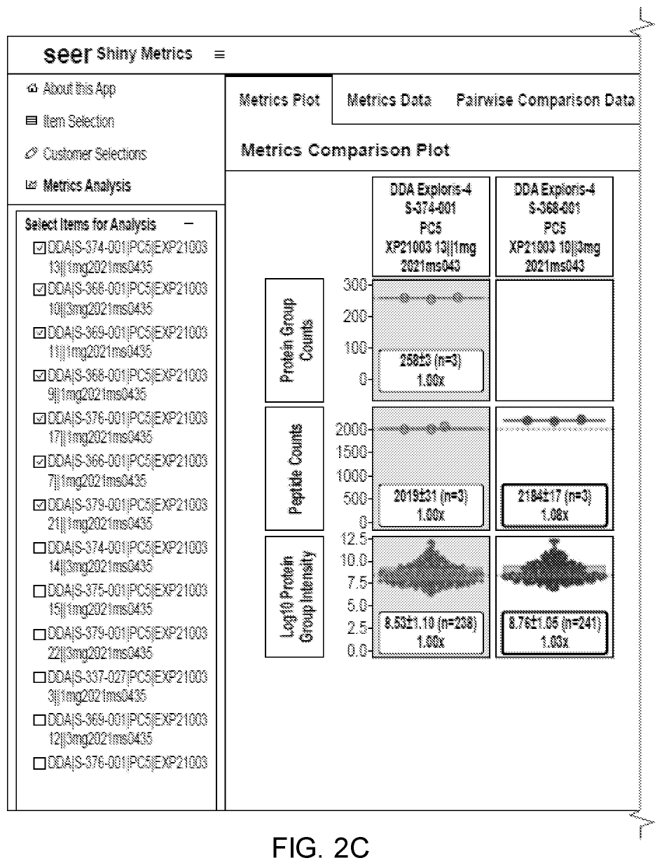

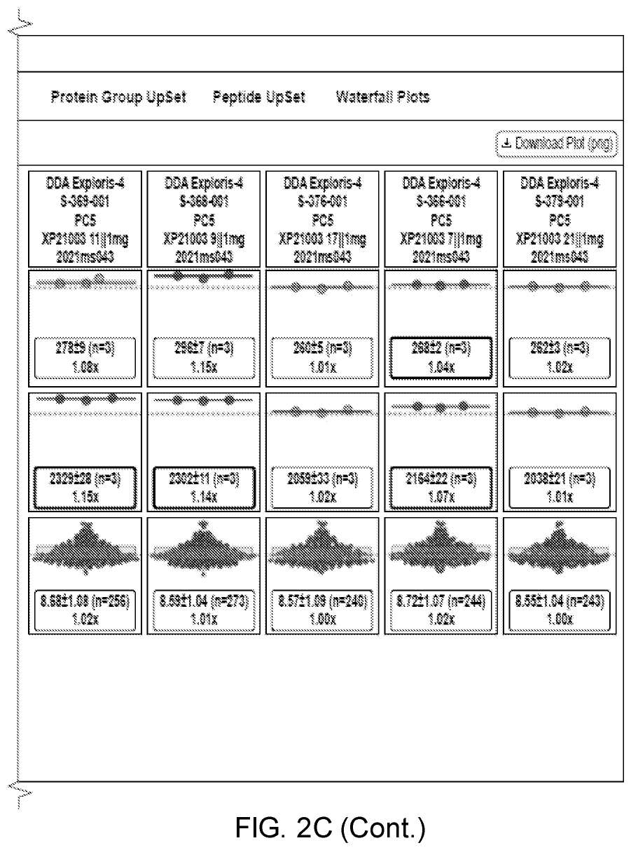

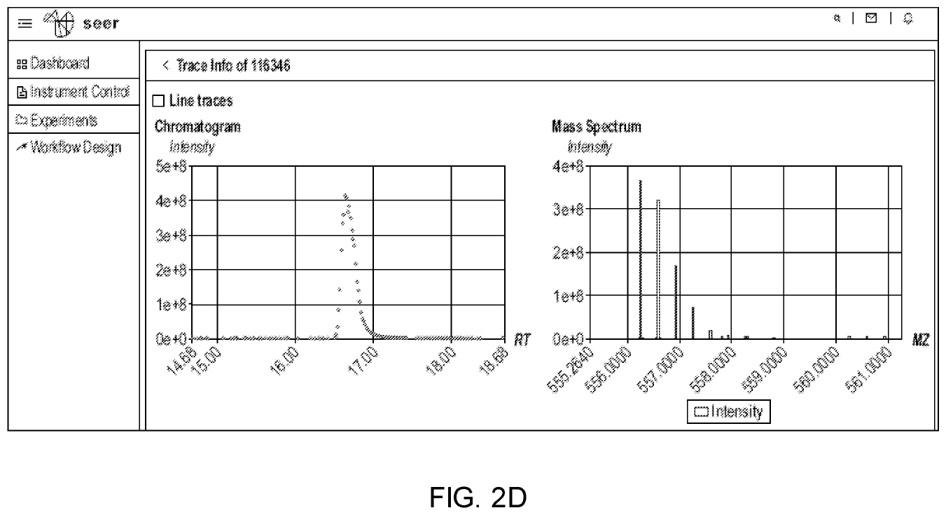

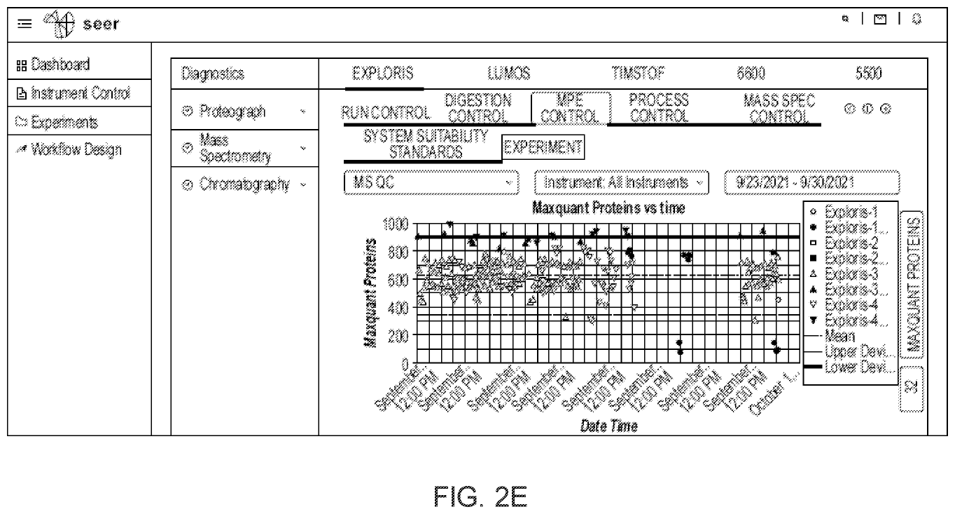

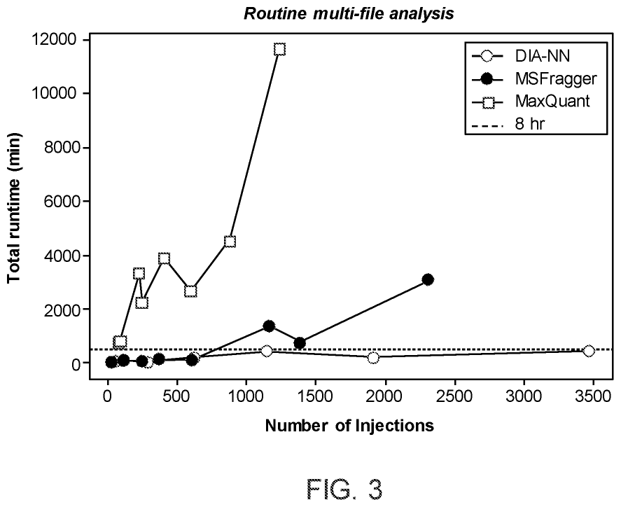

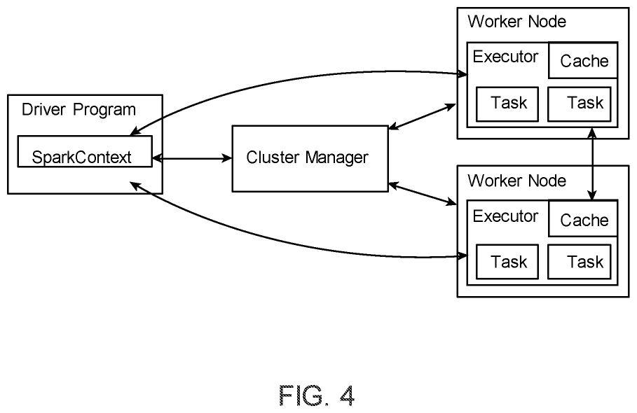

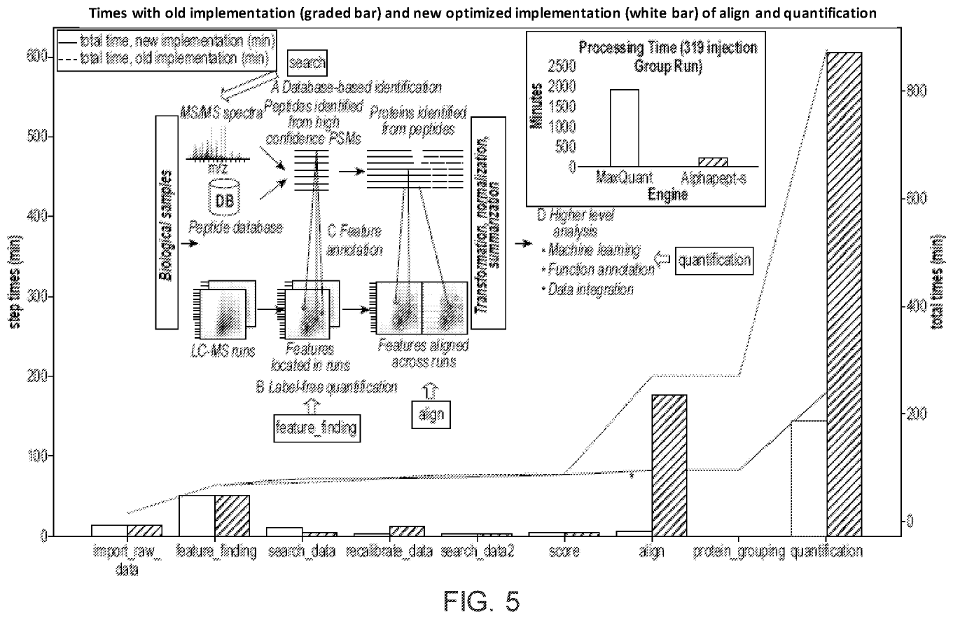

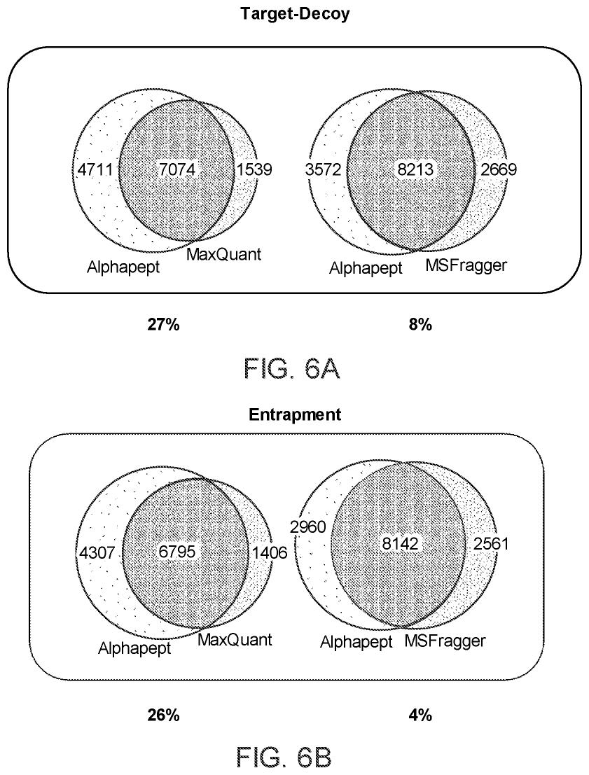

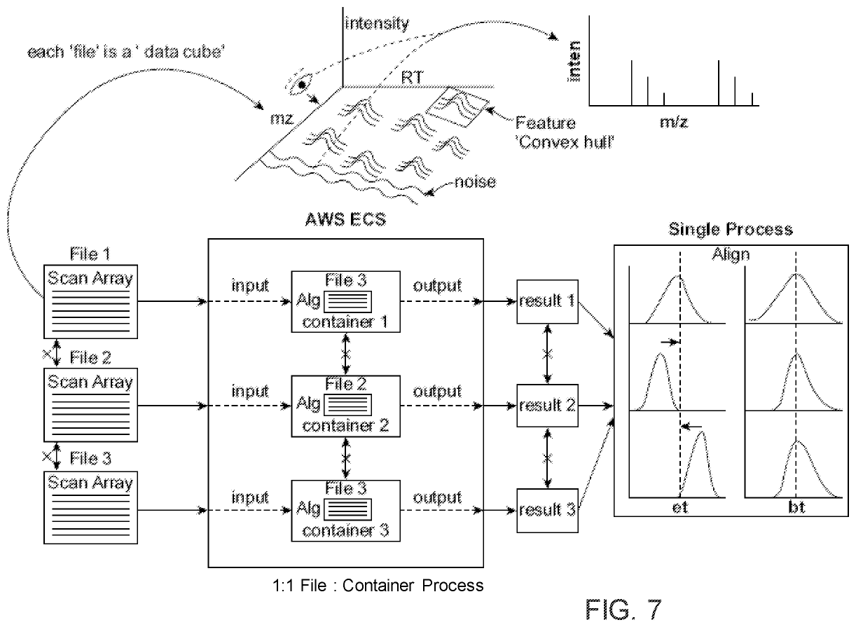

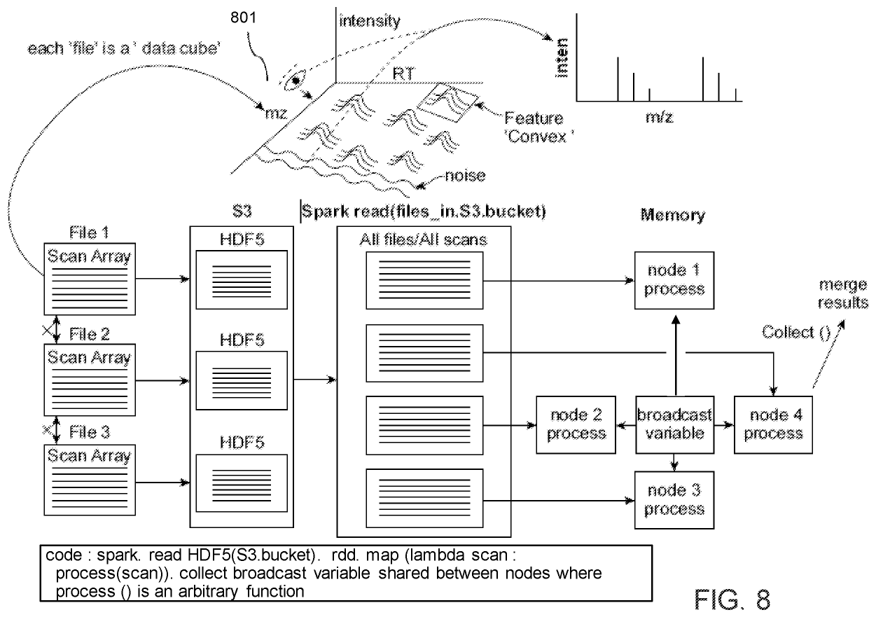

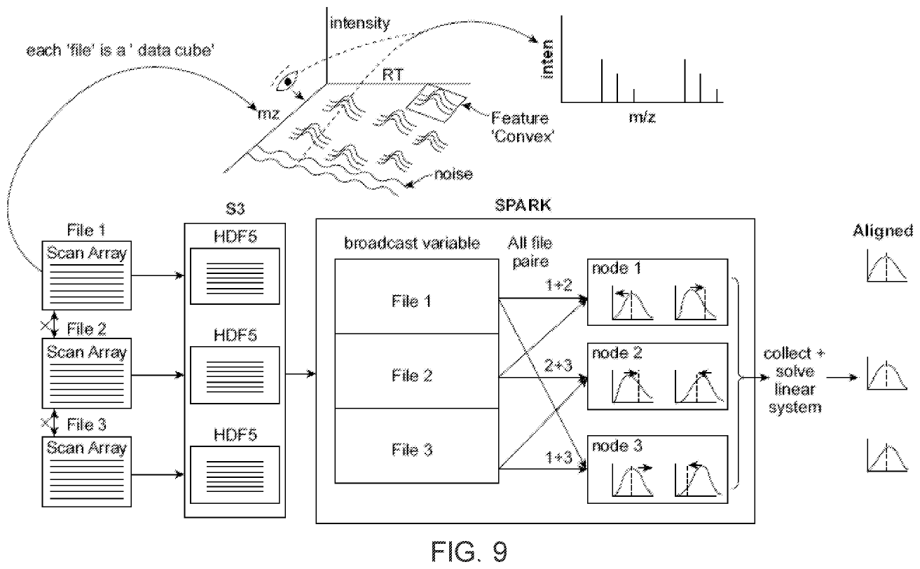

The computer-implemented system can comprise one or more databases. A database can be a distributed relational database (201). A database can be an object-based distributed database (202). A database can be on a server. A database can be a non-relational database (203). A database can be public database, a shared database between two or more entities, or a private database only accessible by one entity. The computer-implemented system can comprise an application programming interface (API) or a GUI. FIGS. 2A-2E schematically illustrates an GUI for a cloud scalable omics data analysis pipeline, in accordance with some embodiments. An API or GUI can track the progress of experiments (e.g., plate information) and data processing routines. For instance, FIG. 2B schematically illustrates a GUI for tracking plate information and analysis for an experiment, in accordance with some embodiments. An API or GUI can be used to generate or visualize metrics for experiments and data processing routines. FIG. 2C schematically illustrates a GUI for generating sample metrics for an experiment, in accordance with some embodiments. An API or GUI can be used to generate or visualize traces. FIG. 2D schematically illustrates a GUI for displaying a trace of an MS feature extracted from a MS dataset from an experiment, in accordance with some embodiments. An API or GUI can be used to generate or visualize metrics for experiment results from multiple instruments, experiments, or both. FIG. 2E schematically illustrates a GUI for viewing analysis results chronologically from multiple experiments conducted on multiple instruments, in accordance with some embodiments. The API or the GUI can be programmed de novo, reprogrammed, or reconfigured by a user to perform new functions.

In some embodiments, the processing further comprises identifying a biomarker in the plurality of harmonized mass spectrometry datasets. In some embodiments, the plurality of harmonized mass spectrometry datasets are differential in at least one clinically relevant dimension. In some embodiments, the biomarker is associated with the at least one clinically relevant dimension. In some embodiments, the processing further comprises performing a power curve analysis based on the plurality of harmonized mass spectrometry datasets. In some embodiments, the power curve analysis provides a statistical power for identifying a biomarker based on the plurality of harmonized mass spectrometry datasets. In some embodiments the power curve analysis provides a ratio between a number of samples to a number of potential biomarkers that can be found with a predetermined statistical significance value. In some embodiments, the processing further comprises training a machine learning model based on the plurality of harmonized mass spectrometry datasets. In some embodiments, the processing further comprises performing clustering analysis based on the plurality of harmonized mass spectrometry datasets. The biomarker can comprise a level of a signal for a biomolecule in a subset in a fraction of the plurality of harmonized mass spectrometry datasets. The biomarker can comprise levels for a plurality of signals for a plurality of biomolecules in a subset in a fraction of the plurality of harmonized mass spectrometry datasets.

### Normalizing Mass Spectrometry Datasets

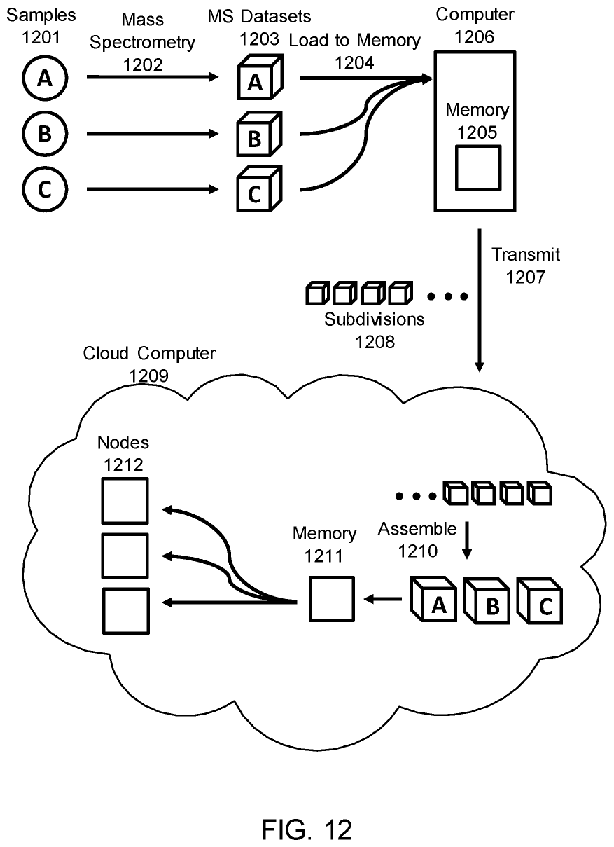

In some aspects, the present disclosure provides a computer-implemented method for normalizing and processing mass spectrometry datasets. FIG. 12 schematically illustrates a computer-implemented method for transmitting harmonized mass spectrometry datasets between computing nodes, in accordance with some embodiments. The computer-implemented method can comprise obtaining a plurality of mass spectrometry datasets (1203) obtained from a plurality of samples (1201). The plurality of mass spectrometry datasets can be obtained by performing mass spectrometry (1202) on the plurality of samples. The plurality of mass spectrometry datasets can comprise a plurality of harmonized mass spectrometry datasets. In some embodiments, the harmonized dataset are obtained through the method of storing and processing mass spectrometry datasets discussed above. For example, mass spectrometry datasets are converted to a plurality of harmonized mass spectrometry datasets as depicted FIG. 1A. In some embodiments, the computer-implemented method comprises loading (1204) the plurality of mass spectrometry datasets into a memory (1205) of a computing node (1206) to generate a cached dataset. The computer-implemented method can comprise transmitting (1207) a copy of the cached dataset (1208) to a plurality of cache memories of a plurality of computing nodes (1212). The transmitting can be performed using one or more of a variety of wired and/or wireless connections. In some embodiments, the computer-implemented method comprises determining, using the plurality of computing nodes, a plurality of feature values for the plurality of mass spectrometry datasets. The computer-implemented method can comprise normalizing, using the plurality of computing nodes, across the plurality of mass spectrometry datasets using the plurality of feature values to generate a plurality of normalized mass spectrometry datasets. In some embodiments, the computer-implemented method comprises processing the plurality of normalized mass spectrometry datasets to compare the plurality of samples.

In some embodiments, the plurality of mass spectrometry datasets (1203) comprises a set of precursors for each sample in the plurality of samples. In some embodiments, the set of precursors comprises a set of biomolecule precursors. In some embodiments, the set of biomolecule precursors comprises a set of polyamino acid precursors.

In some embodiments, the plurality of mass spectrometry datasets (1203) comprises information about a single cell, a tissue, an organ, a system of tissues and/or organs (such as cardiovascular, respiratory, digestive, or nervous systems), or an entire multicellular organism. In some embodiments, the plurality of mass spectrometry datasets comprises information about an individual (e.g., an individual human being or an individual bacterium), or a population of individuals (e.g., human beings with diagnosed with cancer or a colony of bacteria). The plurality of mass spectrometry datasets may comprise information from various forms of life, including forms of life from the Archaea, the Bacteria, the Eukarya, the Protozoa, the Chromista, the Plantae, the Fungi, or from the Animalia. In some embodiments, the plurality of mass spectrometry datasets may comprise information from viruses.

In some embodiments, the plurality of mass spectrometry datasets (1203) comprises a set of chemical identifications for each sample in the plurality of samples. In some embodiments, the set of chemical identifications comprises a set of biomolecule identifications. In some embodiments, the set of biomolecule identifications comprises a set of polyamino acid identifications. In some embodiments, the set of polyamino acid identifications comprises a set of tryptic or semi-tryptic peptide identifications. In some embodiments, the plurality of mass spectrometry datasets comprises a set of chemical intensities for each sample in the plurality of samples. In some embodiments, the set of chemical intensities comprises a set of biomolecule intensities. In some embodiments, the set of biomolecule intensities comprises a set of polyamino acid intensities. In some embodiments, the set of polyamino acid intensities comprises a set of tryptic or semi-tryptic peptide intensities. In some embodiments, the set of polyamino acid identifications comprises a set of protein group identifications. In some embodiments, the set of polyamino acid intensities comprises a set of protein group intensities.

In some embodiments, the plurality of mass spectrometry datasets (1203) comprises a data independent acquisition (DIA) mass spectrometry dataset, a data dependent acquisition (DDA) mass spectrometry dataset, or both. In some embodiments, the plurality of mass spectrometry datasets comprises a LC-MS dataset, a LC-MS/MS dataset, or both. The mass spectrometry (1202) can comprise a LC-MS dataset, a LC-MS/MS dataset, or both. The mass spectrometry can be performed with DIA, DDA, or both.

As discussed further below, the plurality of mass spectrometry datasets (1203) may be derived, for example, from biological samples (e.g., plasma, etc.). In addition, the plurality of mass spectrometry datasets (1203) may be derived, for example, from samples where biomolecules, such as peptides or proteins, have been selectively enriched. In addition, the plurality of mass spectrometry datasets (1203) may be derived, for example, from samples where non-specific binding to surfaces (e.g., to two or more different nanoparticles have different physicochemical properties) has been used to compress the dynamic range of the sample.

In some embodiments, the computing node (1206) is a local computing node. In some embodiments, the local computing node comprises a computing device interfacing with a user. In some embodiments, a desktop computer, a laptop computer, or a mobile device comprises the local computing node. In some embodiments, an instrument comprises the local computing node. In some embodiments, a mass spectrometry or a sequencing instrument comprises the local computing node. In some embodiments, the computing node comprises a cloud-computing node.

In some embodiments, the plurality of computing nodes (1212) comprises a plurality of cloud-computing nodes. In some embodiments, a cloud-computing cluster comprises one or more cloud-computing nodes. In some embodiments, an instance comprises one or more cloud-computing clusters. In some embodiments, a plurality of computing nodes comprises the computing node. In some embodiments, the plurality of computing nodes comprises at least 2, 5, 10, 100, 1000, 10000, or 100000 computing nodes. In some embodiments, the plurality of computing nodes comprises at most 10, 100, 1000, 10000, 100000, or 1000000 computing nodes. In some embodiments, a cloud computing node comprises a virtual machine instance. The number of nodes in the plurality of nodes can be autonomously scaled based on the size or amount of the mass spectrometry datasets, the complexity of the task to be performed using the mass spectrometry datasets, or both.

In some embodiments, the memory (1205) comprises a random access memory (RAM). In some embodiments, the memory comprises a cache memory. In some embodiments, the cache memory may comprise a level 1, level 2, level 3, level 4 cache memory, or any combination thereof. In some embodiments, the cache memory may comprise at least 32 kilobytes (KB), 64 KB, 128 KB, 256 KB, 512 KB, 1 megabyte (MB), 2 MB, 4 MB, 8 MB, 16 MB, 32 MB, 64 MB, 128 MB, 256 MB, 512 MB, 1 gigabyte (GB), 2 GB, 4 GB, 8 GB, 16 GB, 32 GB, 64 GB, 128 GB, 256 GB, or 512 GB. In some embodiments, the cache memory may comprise at most 32 kilobytes (KB), 64 KB, 128 KB, 256 KB, 512 KB, 1 megabyte (MB), 2 MB, 4 MB, 8 MB, 16 MB, 32 MB, 64 MB, 128 MB, 256 MB, 512 MB, 1 gigabyte (GB), 2 GB, 4 GB, 8 GB, 16 GB, 32 GB, 64 GB, 128 GB, 256 GB, or 512 GB. In some embodiments, a plurality of cache memories comprises the cache memory. In some embodiments, a plurality of computing nodes may comprise the plurality of cache memories. In some embodiments, the plurality of cache memories can be in operable communication with a plurality of buses for transmitting or receiving data. The transmitting or receiving can be performed using one or more of a variety of wired and/or wireless connections. The plurality of buses can comprise various protocols and technologies, including Modem, LTE, GSM, DOCSIS, OC, Ethernet, Infiniband, IEEE 802.11, Bluetooth, for example. The plurality of buses can comprise a bit rate of at least 32 kilobytes (KB), 64 KB, 128 KB, 256 KB, 512 KB, 1 megabyte (MB), 2 MB, 4 MB, 8 MB, 16 MB, 32 MB, 64 MB, 128 MB, 256 MB, 512 MB, 1 gigabyte (GB), 2 GB, 4 GB, 8 GB, 16 GB, 32 GB, 64 GB, 128 GB, 256 GB, or 512 GB per second. The plurality of buses can comprise a bit rate of at most 32 kilobytes (KB), 64 KB, 128 KB, 256 KB, 512 KB, 1 megabyte (MB), 2 MB, 4 MB, 8 MB, 16 MB, 32 MB, 64 MB, 128 MB, 256 MB, 512 MB, 1 gigabyte (GB), 2 GB, 4 GB, 8 GB, 16 GB, 32 GB, 64 GB, 128 GB, 256 GB, or 512 GB per second.

In some embodiments, the cached dataset is an unserialized cached dataset. In some embodiments, the unserialized cached dataset is serialized to generate a serialized cached dataset. In some embodiments, the serialized cached dataset comprises a series of bytes. In some embodiments, the serialized cached dataset is subdivided to generate a subdivided cached dataset. In some embodiments, the subdivided cached dataset may comprise a plurality of subdivisions. In some embodiments, a subdivision may comprise at least 8 bytes (B), 16 B, 32 B, 64 B, 128 B, 256 B, 512 B, 1 kB, 2 kB, 4 kB, 8 kB, 16 kB, 32 kB, 64 kB, 128 kB, 256 kB, 512 kB, 1 MB, 2 MB, 4 MB, 8 MB, 16 MB, 32 MB, 64 MB, 128 MB, 256 MB, 512 MB, or 1 GB.

In some embodiments, the transmitting (1207) comprises transmitting the plurality of subdivisions of the subdivided cached dataset. In some embodiments, the plurality of subdivisions are transmitted one subdivision at a time. In some embodiments, the plurality of subdivisions are transmitted more than one subdivision at a time. In some embodiments, the transmitting comprises assembling a copy of the serialized cached dataset from the copy of the subdivided cache. In some embodiments, the copy of the serialized cached dataset is assembled at a computing node in the plurality of computing nodes.

The plurality of mass spectrometry datasets (1203) can be a plurality of harmonized mass spectrometry datasets. The plurality of mass spectrometry datasets can comprise a columnar format. The plurality of mass spectrometry datasets can be stored on a distributed storage system. The plurality of mass spectrometry datasets can be stored on an object-based storage system. The plurality of mass spectrometry datasets can be stored on a distributed relational storage system. The plurality of mass spectrometry datasets can be stored on a non-relational storage system. The plurality of mass spectrometry datasets can be stored on a public storage system, a shared storage system between two or more entities, or a private storage system.

The amount of time that it takes to process a mass spectrometry dataset can be significantly reduced. In some embodiments, a processing time for one or more processes of the computer-implemented method may be substantially linear as a function of a number of mass spectrometry datasets in the plurality of mass spectrometry datasets. In some embodiments, performing for one or more processes of the computer-implemented method may take less than ax^{1.8}, ax^{1.6}, ax^{1.4}, or ax^{1.2} amount of compute time, wherein x is a number of mass spectrometry datasets in the plurality of mass spectrometry datasets, and wherein a is a constant. In some embodiments, performing for one or more processes of the computer-implemented method may take less than ax^{1.8}, ax^{1.6}, ax^{1.4}, or ax^{1.2} amount of real time, wherein x is a number of mass spectrometry datasets in the plurality of mass spectrometry datasets, and wherein a is a constant.

In some embodiments, the processing further comprises determining a biomarker in the plurality of mass spectrometry datasets. In some embodiments, the processing further comprises determining a biomarker based on the plurality of normalized mass spectrometry datasets. In some embodiments, the plurality of samples are differential in at least one clinically relevant dimension. In some embodiments, the biomarker is associated with the at least one clinically relevant dimension. In some embodiments, the processing further comprises performing a power curve analysis based on the plurality of normalized mass spectrometry datasets. In some embodiments, the power curve analysis provides a statistical power for identifying a biomarker based on the plurality of normalized mass spectrometry datasets. In some embodiments the power curve analysis provides a ratio between a number of samples to a number of potential biomarkers that can be found with a predetermined statistical significance value. In some embodiments, the processing further comprises training a machine learning model based on the plurality of normalized mass spectrometry datasets. In some embodiments, the processing further comprises performing clustering analysis based on the plurality of normalized mass spectrometry datasets. The biomarker can comprise a level of a signal for a biomolecule in a subset in a fraction of the plurality of mass spectrometry datasets. The biomarker can comprise levels for a plurality of signals for a plurality of biomolecules in a subset in a fraction of the plurality of mass spectrometry datasets.

### Alignment

In some embodiments, a method of the present disclosure may comprise normalizing, using a plurality of computing nodes, across a plurality of mass spectrometry datasets using a plurality of feature values to generate a plurality of normalized mass spectrometry datasets. In some embodiments, the plurality of mass spectrometry datasets may be normalized such that a chemical identification from one mass spectrometry dataset in the plurality of mass spectrometry datasets may be used to identify another chemical in another mass spectrometry dataset in the plurality of mass spectrometry datasets. In some embodiments, a feature value may be applied to a mass spectrometry dataset in a relative fashion (i.e., applied to mass-to-charge ratio and mobility) or in an absolute fashion (i.e., applied to retention time).

In some embodiments, the aligning may be based on a plurality of feature values. In some embodiments, the plurality of feature values comprises a feature value for the set of precursors of each mass spectrometry dataset in the plurality of mass spectrometry datasets. In some embodiments, the feature value is configured for normalizing retention time, mass-to-charge ratio, ion mobility, or a combination thereof. In some embodiments, the feature value is a shifting value. In some embodiments, the shifting value is added to the retention time, mass-to-charge ratio, or ion mobility for a mass spectrometry dataset in the plurality of mass spectrometry datasets.

In some embodiments, the feature values are based on isotopic clusters. In some embodiments, the feature values comprise retention time, mass-to-charge ratio, aggregate peak area of the isotope cluster, ion mobility, or any combination thereof. In some embodiments, the normalizing generates a set of aligned precursors for each mass spectrometry dataset in the plurality of mass spectrometry datasets. In some embodiments, the normalizing further comprises identifying a first chemical from a first mass spectrometry dataset in the plurality of mass spectrometry datasets based on an aligned precursor in the set of aligned precursors of a second mass spectrometry dataset.

In some embodiments, the determining comprises minimizing an objective function, using a computing node in the plurality of computing nodes, based on a pair of mass spectrometry datasets in the plurality of mass spectrometry datasets. In some embodiments, the determining comprises minimizing the objective function for a unique pair of mass spectrometry datasets in the plurality of mass spectrometry datasets for each computing node in the plurality of computing nodes.

### Quantification

In some embodiments, a method of the present disclosure may comprise normalizing, using a plurality of computing nodes, across a plurality of mass spectrometry datasets using a plurality of feature values to generate a plurality of normalized mass spectrometry datasets. In some embodiments, the normalizing may be performed to determine intensities of chemicals in the plurality of mass spectrometry datasets. In some embodiments, the intensities of chemicals may be determined such that comparisons can be made between individual mass spectrometry datasets in the plurality of mass spectrometry datasets. In some embodiments, the normalizing comprises label-free quantification. In some embodiments, the normalizing generates a set of relative abundances for each mass spectrometry dataset in the plurality of mass spectrometry datasets.

In some embodiments, a feature value in the plurality of feature values may be determined by minimizing an objective function, using a computing node in the plurality of computing nodes, based on a pair of mass spectrometry datasets in the plurality of mass spectrometry datasets. In some embodiments, the objective function is minimized for a unique pair of mass spectrometry datasets in the plurality of mass spectrometry datasets for each computing node in the plurality of computing nodes.

In some embodiments, the objective function comprises:

$$L = {\sum }_{p}^{N} \vert \frac{I ( {Norm}_{A} , p )}{I ( {Norm}_{B} , p )} \vert ,$$

wherein N is a number of chemical identifications in the set of chemical identifications, wherein p is a chemical in the set of chemical identifications, wherein I is an intensity value for the set of chemical intensities, wherein Norm_{A} is a first feature value for a first mass spectrometry dataset in the pair of mass spectrometry datasets, and wherein Norm_{B} is a second feature value for a second mass spectrometry dataset in the pair of mass spectrometry datasets.

In some embodiments, the objective function comprises:

$$L = {\sum }_{A , B}^{M} {\sum }_{p}^{N} \vert \frac{I ( {Norm}_{A} , p , A )}{I ( {Norm}_{B} , p , B )} \vert ,$$

wherein M is a number of unique pairs of mass spectrometry datasets in the plurality of mass spectrometry datasets, and wherein A,B is the unique pair of mass spectrometry datasets in the plurality of mass spectrometry datasets.

In some embodiments, the set of relative abundances comprises a set of chemical relative abundances. In some embodiments, the set of chemical relative abundances comprises a set of biomolecule relative abundances. In some embodiments, the set of biomolecule relative abundances comprises a set of polyamino acid relative abundances. In some embodiments, the set of relative abundances represent relative abundances of chemicals between the plurality of mass spectrometry datasets. In some embodiments, the set of relative abundances represent relative abundances of polyamino acids between the plurality of mass spectrometry datasets. In some embodiments, the plurality of feature values comprises a feature value for the set of chemical intensities of each mass spectrometry dataset in the plurality of mass spectrometry datasets. In some embodiments, the normalizing comprises adjusting the set of chemical intensities for each mass spectrometry dataset in the plurality of mass spectrometry datasets based on the plurality of feature values.

### Method for Determining Expressed Proteoforms

In some aspects, the present disclosure describes a method for assaying a biological sample. In some cases, the method comprises assaying a set of nucleic acids from the biological sample to obtain genotypic information of the biological sample. In some cases, the method comprises generating, based at least in part on the genotypic information, a set of expressible proteoforms that can be expressed from the set of nucleic acids. In some cases, the method comprises assaying a set of polyamino acids from the biological sample to generate proteomic information of the biological sample. In some cases, the method comprises mapping the set of identifications to the set of expressible proteoforms, thereby determining a set of expressed proteoforms in the biological sample. In some cases, the proteomic information comprises a set of identifications for the set of polyamino acids.

In some cases, a biological sample may comprise various biomolecules, including proteins, nucleic acids, lipids, carbohydrates, any combination thereof, and more. In some cases, the presence or absence and/or concentration of various biomolecules, as well as correlations between various subsets of biomolecules (e.g., proteins and nucleic acids), may be indicative of the biological state of a given biological sample (e.g., a healthy or a disease state). In some cases, the method may be performed with a plurality of biological samples. In some cases, a biological sample may be obtained from a subject. In some cases, a biological sample may be obtained from a plurality of subjects.

In some cases, a nucleic acid may comprise any one of various species or type of nucleic acids. In some cases, a nucleic acid may be single-stranded, double-stranded. In some cases, a nucleic acid may comprise a single-stranded portion and a double-stranded portion. In some cases, a nucleic acid may be linear, branched, or cyclic. In some cases, a nucleic acid may comprise various secondary structures, tertiary structures, or quaternary structures. In some cases, a nucleic acid may comprise a deoxyribonucleic acid (DNA) or ribonucleic acid (RNA). In some case, a nucleic acid may comprise a coding sequence, a non-coding sequence, or both. In some cases, a nucleic acid may comprise a coding or non-coding region of a gene or gene fragment, or any combination thereof. In some cases, a nucleic acid may comprise a messenger ribonucleic acid (mRNA), a DNA, a micro ribonucleic acid (miRNA), a transfer ribonucleic acid (tRNA), a long non-coding RNA (lncRNA), a ribosomal ribonucleic acid (rRNA), a small nuclear RNA (snRNA), a piwi-interacting RNA (piRNA), a small nucleolar RNA (snoRNA), an extracellular RNA(exRNA), a small cajal body-specific RNA (scaRNA), a silencing ribonucleic acid (siRNA), self-amplifying RNA (saRNA), a YRNA (small noncoding RNA), a heterogeneous nuclear RNA (HnRNA), complementary DNA (cDNA), a short-hairpin RNA (shRNA), a ribozyme, a recombinant nucleic acid, a plasmid, a vector, an isolated DNA, an isolated RNA, or any combination thereof.

In some cases, the set of polyamino acids comprises a set of proteins expressed in the biological sample. In some cases, the set of polyamino acids comprises a set of peptide fragments derived from a set of proteins expressed in the biological sample. In some cases, the set of peptide fragments is derived by trypsinization. In some cases, the set of peptide fragments comprise tryptic peptide fragments, semi-tryptic peptide fragments, or both. In some cases, the set of peptide fragments is derived by lysinization. In some cases, the proteomic information comprises a set of peptide intensities detected from assaying the set of polyamino acids. In some cases, the set of identifications comprises protein group identifications for the set of polyamino acids. In some cases, the set of identifications comprises amino acid sequences for the set of polyamino acids. In some cases, the set of identifications comprises mass spectrometry signals for the set of polyamino acids. In some cases, the set of identifications comprises mass spectrometry signals for the set of protein sequencing reads. In some cases, the set of identifications comprises post-translational modifications for the set of polyamino acids. In some cases, the mapping comprises matching an identification in the set of identifications to an expressible proteoform in the set of expressible proteoforms, thereby determining that the matched expressible proteoform is an expressed proteoform in the biological sample. In some cases, the set of expressed proteoforms comprises at least about 10, 20, 30, 40, 50, 60, 70, 80, 90, 100, 200, 300, 400, 500, 600, 700, 800, 900, or 1,000 expressed proteoforms. In some cases, the set of expressed proteoforms comprises at most about 10, 20, 30, 40, 50, 60, 70, 80, 90, 100, 200, 300, 400, 500, 600, 700, 800, 900, or 1,000 expressed proteoforms. In some cases, the set of expressed proteoforms comprises about 10-1000, 20-900, 30-800, 40-700, 50-600, 60-500, 70-400, 80-300, 90-200, or 100-150 expressed proteoforms.

In some cases, the method for assaying a biological sample comprises associating the set of expressed proteoforms with a biological state of the biological sample. In some cases, the associating is based at least partially on the expression levels of each proteoform in the set of expressed proteoforms. In some cases, the associating is based at least partially on the relative abundances of each proteoform in the set of expressed proteoforms. In some cases, the method for assaying a biological sample further comprises associating the genotypic information with the biological state of the biological sample. In some cases, the set of polyamino acids are derived from the biological sample using at least one untargeted assay. In some cases, the set of polyamino acids comprise a dynamic range of at least 5, 6, 7, 8, 9, 10, 11, or 12 orders of magnitude in the biological sample. In some cases, the proteomic information comprises a dynamic range of at least 5, 6, 7, 8, 9, 10, 11, or 12 orders of magnitude in the biological sample. In some cases, the proteomic information comprises a dynamic range of at most 5, 6, 7, 8, 9, 10, 11, 12, 13, 14, or 15 orders of magnitude in the biological sample.

In some cases, the method may further comprise at least one untargeted assay. In some cases, the at least one untargeted assay comprises providing a plurality of surface regions comprising a plurality of surface types. In some cases, the at least one untargeted assay comprises contacting the biological sample with the plurality of surface regions to yield a set of adsorbed biomolecules on the plurality of surface regions. In some cases, the at least one untargeted assay comprises desorbing, from the plurality of surface regions, at least a portion of the set of adsorbed biomolecules to yield the set of polyamino acids. In some cases, the plurality of surface regions is disposed on a single continuous surface. In some cases, the plurality of surface regions is disposed on a plurality of discrete surfaces. In some cases, the plurality of discrete surfaces is surfaces of a plurality of particles. In some cases, the at least one untargeted assay has a false discovery rate of at most about 5%, 4%, 3%, 2%, 1%, 0.9%, 0.8%, 0.7%, 0.6%, 0.5%, 0.4%, 0.3%, 0.2%, or 0.1%. In some cases, the at least one untargeted assay has a false discovery rate of about 5%-0.1%, 4%-0.2%, 3%-0.3%, 2%-0.4%, 1%-0.5%, 0.9%-0.6%, or 0.8%-0.7%. In some cases, the at least one untargeted assay has a false discovery rate of no more than about 5%, 4%, 3%, 2%, 1%, 0.9%, 0.8%, 0.7%, 0.6%, 0.5% 0.4%, 0.3%, 0.2%, or 0.1%.

In some cases, the plurality of surface types may comprise a surface that is configured to capture or interact with a biomolecule from a sample. For example, the surface may be functionalized with nucleic acid binding moieties, such as single stranded nucleic acids, which are capable of binding nucleic acids from a sample.

In some cases, the plurality of surface types may comprise a surface of a particle. In some cases, the particle is a nanoparticle. In some cases, the particle is a microparticle. In some cases, the particle is a bead. In some cases, a particle may be surface functionalized.

In some aspects, the present disclosure describes a method for assaying a biological sample. In some cases, the method comprises assaying a set of peptides from the biological sample using spectral data to generate proteomic information of the biological sample. In some cases, the method comprises identifying a set of protein groups based at least in part on the spectral data of the set of peptides. In some cases, the method comprises identifying one or more sets of peptides that are correlated in abundance for a given protein group in the set of protein groups. In some cases, the method comprises mapping the set of peptides a database of human genes with isoform information, thereby determining a set of proteoforms that result in the set of peptides. In some cases, biological samples may be complex mixtures of various biomolecules, including proteins, nucleic acids, lipids, polysaccharides, and more. In some cases, the one or more samples may comprise one or more biological samples. In some cases, the one or more samples may be obtained from a subject. In some cases, the one or more samples may be obtained from a plurality of subjects. In some cases, the proteomic information comprises a set of identifications for the set of peptides.

In some cases, the spectral data comprises mass spectrometry data. In some cases, the mass spectral data are obtained from the biological sample contacting a plurality of surface types. In some cases, the plurality of surface types may comprise a surface that is configured to capture or interact with a biomolecule from a sample. For example, the surface may be functionalized with nucleic acid binding moieties, such as single stranded nucleic acids, which are capable of binding nucleic acids from a sample. In some cases, the plurality of surface types may comprise a surface of a particle. In some cases, the particle is a nanoparticle. In some aspects, the particle is a microparticle. In some cases, the particle is a bead. In some cases, a particle may be surface functionalized.

In some cases, the identifying the one or more sets of peptides comprises computing a correlation of abundances of each peptide across the biological sample. In some cases, the identifying the one or more sets of peptides comprises computing a correlation of abundances of each peptide across a plurality of biological samples or clustering based on peptides' correlations. In some cases, the method for assaying a biological sample further comprises, subsequent to (c), identifying a first set of peptides that are correlated in abundance; identifying a second set of peptides that are correlated in abundance; and applying a filtering step to confirm that the set of peptides are distinct from each other. In some cases, the method further comprises identifying more than two sets of peptides that are correlated in abundance, and applying a filtering step to confirm that the more than two sets of peptides are distinct from each other. In some cases, the first set of peptides comprise a first proteoform, and the second set of peptides comprise a second proteoform, wherein the first proteoform and the second proteoform are expressed from a same locus of exons. In some cases, the first set of peptides comprise a first proteoform, and the second set of peptides comprise a second proteoform, wherein the first proteoform and the second proteoform are expressed from a same locus of exons. In some cases, the biological sample comprises a plasma sample derived from a subject afflicted with a non-small cell lung cancer. In some cases, an identified proteoform is associated with a disease. In some cases, the set of proteoforms comprise peptide variants, protein variants, or both. In some cases, the set of proteoforms comprise splicing variants, allelic variants, post-translation modification variants, or any combination thereof. In some cases, the database of human genes comprises an ENSEMBL database with isoform information.

In some cases, the methods described herein include identifying proteins with distinct proteoforms. In some cases, proteoform detection in deep plasma preteomics is performed by peptide expression correlation method and genomic mapping. In some cases, the peptide abundances are calculated by the correlation method within each protein group. In some cases, the correlation method is selected from the group consisting of, but is not limited to, the Pearson pairwise correlation, the Kendall rank correlation, the Spearman correlation, the chatterjee correlation, the Point-Biserial correlation, and the like. In some cases, for the identification of clusters of similar abundant peptides, an optimal number of clusters is determined. In some cases, a silhouette method is applied to obtain an optimal number of clusters and K-means clustering on the correlation of peptide abundances is used. In some cases, the method for determining an optimal number of clusters is used in combination with clustering algorithms that requires the specification of number of clusters. In some cases, the method of determining optimal number of clusters is selected from the group consisting of, but is not limited to, Gap statistics, the Elbow Method, Calinski-Harabasz Index, Davies-Bouldin Index, the use of Dendrogram, Bayesian information criterion, and the like. In some cases, the clustering method is selected from the group consisting of, but is not limited to, any centroid-based clustering like K-means, K-medoid, k-modes, k-median, and the like. In some cases, clustering algorithm that requires no specification of number of clusters is used to cluster peptides. In some cases, the method to cluster peptides into groups for proteoform identification is selected from the group consisting of, but is not limited to, Density-based Clustering like DBSCAN and DENCAST, Distribution-based Clustering like Gaussian Mixed Models and DBCLASD, and hierarchical clustering like DIANA and AGNES.

In some cases, a filtering step is applied to ensure that the quantitative profile of peptides from different clusters are distinct. In some cases, the filtering step comprises calculating inter-cluster correlations between peptides within a cluster and peptides outside of a cluster. In some cases, the average of all inter-cluster correlations is lower than a certain threshold for the protein to be designated as a protein with distinct clusters. In some cases, the threshold is calculated based on the distribution of correlation of all proteins in the cohort, one standard deviation lower than the mean of the distribution can be used as the threshold. In some cases, peptides are mapped to protein isoforms from the ENSEMBL database as a separate process. In some cases, the presence of a proteoform is inferred if the known protein isoform explains the results of the peptide clustering.

### Method for Determining Expression Patterns of Genes

In some aspects, the present disclosure describes a method for assaying a biological sample. In some cases, the method comprises assaying a set of polyamino acids from the biological sample to generate proteomic information of the biological sample. In some cases, the proteomic information comprises a set of identifications for the set of polyamino acids. In some cases, the method comprises assaying a set of nucleic acids from the biological sample to obtain genotypic information of the biological sample. In some cases, the genotypic information comprises one or more nucleic acid sequences. In some cases, the method comprises determining an expression pattern of one or more regions in the one or more nucleic acid sequences. In some cases, the determining is based at least partially on the set of identifications.

In some cases, an expression pattern may comprise expression levels of polyamino acids associated with the one or more regions in the one or more nucleic acid sequences. In some cases, an expression pattern may comprise expression levels of polyamino acids associated with DNA associated with the one or more regions in the one or more nucleic acid sequences. In some cases, an expression pattern may comprise expression levels of polyamino acids associated with pre-mRNA associated with the one or more regions in the one or more nucleic acid sequences. In some cases, an expression pattern may comprise expression levels of polyamino acids associated with mRNA associated with the one or more regions in the one or more nucleic acid sequences. In some cases, an expression pattern may comprise expression levels of pre-mRNA associated with the one or more regions in the one or more nucleic acid sequences. In some cases, an expression pattern may comprise expression levels of mRNA associated with the one or more regions in the one or more nucleic acid sequences. In some cases, an expression pattern may comprise expression levels of at least 1, 2, 3, 4, 5, 6, 7, 8, 9, 10, 20, 30, 40, 50, 60, 70, 80, 90, or 100 polyamino acids. In some cases, an expression pattern may comprise usage patterns of one or more exons in the one or more nucleic acid sequences.

In some cases, an expression pattern may be associated with a disease state. In some cases, an expression pattern may be associated with a prognostic state. In some cases, an expression pattern may be useful as a biomarker. In some cases, an expression pattern may indicate what proteoforms may be expressed from at least a subset of the one or more nucleic acid sequences. In some cases, an expression pattern may indicate regulatory mechanisms that control transcription of at least a subset of the one or more nucleic acid sequences or translation thereof.

In some cases, the proteomic information comprises a set of identifications for the set of polyamino acids. In some cases, the genotypic information comprises one or more nucleic acid sequences. In some cases, the set of polyamino acids comprises a set of peptide fragments derived from a set of proteins expressed in the biological sample. In some cases, the set of identifications comprises protein group identifications or amino acid sequences for the set of polyamino acids. In some cases, the set of nucleic acids is an exome of the biological sample and the genotypic information comprises an exome sequence of the biological sample. In some cases, the one or more regions are one or more exons in the exome sequence. In some cases, the method may comprise determining a nucleic acid sequence with lower error rate based at least partially on the set of identifications of the polyamino acids. In some cases, the method may comprise determining an identification of a polyamino acid with lower error rate based at least partially on a nucleic acid sequence.

In some cases, the set of polyamino acids are derived from the biological sample using at least one untargeted assay. In some cases, the set of polyamino acids comprise a dynamic range of at least 5 orders of magnitude in the biological sample. In some cases, the set of polyamino acids comprise a dynamic range of at least 5, 6, 7, 8, 9, 10, 11, or 12 orders of magnitude in the biological sample. In some cases, the set of polyamino acids comprise a dynamic range of at most 5, 6, 7, 8, 9, 10, 11, or 12 orders of magnitude in the biological sample.

In some cases, the at least one untargeted assay comprises providing a plurality of surface regions comprising a plurality of surface types. In some cases, the at least one untargeted assay comprises contacting the biological sample with the plurality of surface regions to yield a set of adsorbed biomolecules on the plurality of surface regions. In some cases, the at least one untargeted assay comprises desorbing, from the plurality of surface regions, at least a portion of the set of adsorbed biomolecules to yield the set of polyamino acids. In some cases, the plurality of surface regions are disposed on a single continuous surface. In some cases, the plurality of surface regions are disposed on a plurality of discrete surfaces. In some cases, the plurality of discrete surfaces are surfaces of a plurality of particles.

In some cases, the plurality of surface types may comprise a surface that is configured to capture or interact with a biomolecule from a sample. For example, the surface may be functionalized with nucleic acid binding moieties, such as single stranded nucleic acids, which are capable of binding nucleic acids from a sample. In some aspects, the plurality of surface types may comprise a surface of a particle. In some aspects, the particle is a nanoparticle. In some aspects, the particle is a microparticle. In some aspects, the particle is a bead. In other cases, the particle is a synthesized particle. In some cases, a particle may be surface functionalized.

In some cases, the set of polyamino acids comprises a set of proteins expressed in the biological sample. In some cases, the set of polyamino acids comprises a set of peptide fragments derived from a set of proteins expressed in the biological sample. In some cases, the set of peptide fragments is derived by trypsinization. In some cases, the set of peptide fragments is derived by lysinization. In some cases, the proteomic information comprises a set of peptide intensities detected from assaying the set of polyamino acids. In some cases, the set of identifications comprises protein group identifications for the set of polyamino acids. In some cases, the set of identifications comprises amino acid sequences for the set of polyamino acids. In some cases, the set of identifications comprises mass spectrometry signals for the set of polyamino acids. In some cases, the set of identifications comprises post-translational modifications for the set of polyamino acids. In some cases, the mapping comprises matching an identification in the set of identifications to an expressible proteoform in the set of expressible proteoforms, thereby determining that the matched expressible proteoform is an expressed proteoform in the biological sample. In some cases, the set of expressed proteoforms comprises at least about 10, 20, 30, 40, 50, 60, 70, 80, 90, 100, 200, 300, 400, 500, 600, 700, 800, 900, or 1,000 expressed proteoforms. In some cases, the set of expressed proteoforms comprises at most about 10, 20, 30, 40, 50, 60, 70, 80, 90, 100, 200, 300, 400, 500, 600, 700, 800, 900, or 1,000 expressed proteoforms. In some cases, the set of expressed proteoforms comprises about 10-1000, 20-900, 30-800, 40-700, 50-600, 60-500, 70-400, 80-300, 90-200, or 100-150 expressed proteoforms.

In some cases, the method comprises associating the expression pattern with a biological state of the biological sample. In some cases, the associating is based at least partially on the expression levels of each proteoform in the set of expressed proteoforms. In some cases, the associating is based at least partially on the transcription levels of each nucleic acid sequence in the one or more nucleic acid sequences. In some cases, the associating is based at least partially on the relative abundances of each proteoform in the set of expressed proteoforms. In some cases, the method for assaying a biological sample further comprises associating the genotypic information with the biological state of the biological sample. In some cases, the set of polyamino acids are derived from the biological sample using at least one untargeted assay. In some cases, the set of polyamino acids comprise a dynamic range of at least 5, 6, 7, 8, 9, 10, 11, or 12 orders of magnitude in the biological sample. In some cases, the proteomic information comprises a dynamic range of at least 5, 6, 7, 8, 9, 10, 11, or 12 orders of magnitude in the biological sample. In some cases, the proteomic information comprises a dynamic range of at most 5, 6, 7, 8, 9, 10, 11, 12, 13, 14, or 15 orders of magnitude in the biological sample.

In some cases, the method may further comprise at least one untargeted assay. In some cases, the at least one untargeted assay comprises providing a plurality of surface regions comprising a plurality of surface types. In some cases, the at least one untargeted assay comprises contacting the biological sample with the plurality of surface regions to yield a set of adsorbed biomolecules on the plurality of surface regions. In some cases, the at least one untargeted assay comprises desorbing, from the plurality of surface regions, at least a portion of the set of adsorbed biomolecules to yield the set of polyamino acids. In some cases, the plurality of surface regions is disposed on a single continuous surface. In some cases, the plurality of surface regions is disposed on a plurality of discrete surfaces. In some cases, the plurality of discrete surfaces is surfaces of a plurality of particles.

In some cases, the plurality of surface types may comprise a surface that is configured to capture or interact with a biomolecule from a sample. For example, the surface may be functionalized with nucleic acid binding moieties, such as single stranded nucleic acids, which are capable of binding nucleic acids from a sample.

In some cases, the plurality of surface types may comprise a surface of a particle. In some cases, the particle is a nanoparticle. In some cases, the particle is a microparticle. In some cases, the particle is a bead. In some cases, a particle may be surface functionalized.

### Method for Identifying a Differentially Expressed Polyamino Acids

In some aspects, the present disclosure describes a method for identifying a differentially expressed polyamino acid. In some cases, the method comprises obtaining a plurality of polyamino acids from a plurality of biological samples. In some cases, the method comprises assaying the plurality of polyamino acids, using at least one untargeted assay, to generate a plurality of identifications for the plurality of polyamino acids. In some cases, the method comprises identifying at least one polyamino acid in the plurality of polyamino acids that is differentially expressed in the at least one clinically relevant dimension. In some cases, the plurality of biological samples are differential in at least one clinically relevant dimension. In some cases, the plurality of polyamino acids comprises one or more peptide fragments derived from proteins expressed in the plurality of biological samples. In some cases, the at least one clinically relevant dimension is a disease state. In some cases, the disease state is a presence of cancer or an absence of cancer. In some cases, the disease state is a stage of cancer. In some cases, the differentially expressed polyamino acid is upregulated when it is indicative of the disease state. In some cases, the differentially expressed polyamino acid is downregulated when it is indicative of the disease state.

In some cases, the clinically relevant dimension may be a disease state. In some cases, the clinically relevant dimension may comprise a presence or an absence of a disease. In some cases, the clinically relevant dimension may comprise severity of a disease. In some cases, the clinically relevant dimension may comprise a progression of a disease. In some cases, the clinically relevant dimension may comprise a likelihood of recovery by a patient. In some cases, the clinically relevant dimension may comprise a likelihood of success of a therapy or procedure on a patient. In some cases, the clinically relevant dimension may comprise a likelihood of a presence or an absence of a side-effect associated with a therapeutic.

In some cases, the plurality of biological samples may comprise biological samples from a population of individuals. In some cases, the population of individual may comprise a subset of individuals afflicted or suspected of being afflicted with a disease. In some cases, the population of individual may comprise a subset of healthy individuals. In some cases, the population of individuals may comprise individuals at various stages in a disease. In some cases, the population of individuals may comprise males, females, age groups, or any combination thereof. In some cases, the population of individuals may comprise individuals with various diets.

In some cases, the plurality of polyamino acids are peptide fragments derived from proteins expressed in the plurality of biological samples. In some cases, the set of polyamino acids comprise a dynamic range of at least 5 orders of magnitude in the biological sample. In some cases, the set of polyamino acids comprise a dynamic range of at least 6 orders of magnitude in the biological sample. In some cases, the set of polyamino acids comprise a dynamic range of at least 7 orders of magnitude in the biological sample. In some cases, the set of polyamino acids comprise a dynamic range of at least 8 orders of magnitude in the biological sample. In some cases, the set of polyamino acids comprise a dynamic range of at least 9 orders of magnitude in the biological sample. In some cases, the set of polyamino acids comprise a dynamic range of at least 10 orders of magnitude in the biological sample. In some cases, the set of polyamino acids comprise a dynamic range of at least 11 orders of magnitude in the biological sample. In some cases, the set of polyamino acids comprise a dynamic range of at least 12 orders of magnitude in the biological sample. In some cases, the set of polyamino acids comprise a dynamic range of at least 5, 6, 7, 8, 9, 10, 11, or 12 orders of magnitude in the biological sample.

In some cases, the at least one untargeted assay comprises providing a plurality of surface regions comprising a plurality of surface types. In some cases, the at least one untargeted assay comprises contacting the biological sample with the plurality of surface regions to yield a set of adsorbed biomolecules on the plurality of surface regions. In some cases, the at least one untargeted assay comprises desorbing, from the plurality of surface regions, at least a portion of the set of adsorbed biomolecules to yield the set of polyamino acids. In some cases, the plurality of surface regions are disposed on a single continuous surface. In some cases, the plurality of surface regions are disposed on a plurality of discrete surfaces. In some cases, the plurality of discrete surfaces are surfaces of a plurality of particles.

In some cases, the plurality of surface types may comprise a surface that is configured to capture or interact with a biomolecule from a sample. For example, the surface may be functionalized with nucleic acid binding moieties, such as single stranded nucleic acids, which are capable of binding nucleic acids from a sample. In some aspects, the plurality of surface types may comprise a surface of a particle. In some aspects, the particle is a nanoparticle. In some aspects, the particle is a microparticle. In some aspects, the particle is a bead. In other cases, the particle is a synthesized particle. In some cases, a particle may be surface functionalized.

In some cases, the determining comprises identifying one or more base positions in the one or more nucleic acid sequences that covaries with at least one element in the proteomic information. In some cases, the one or more base positions comprise a single nucleotide polymorphism. In some cases, the one or more base positions comprise a deletion or an insertion. In some cases, the one or more base positions comprise a methylation. In some cases, the at least one element comprises a polyamino acid identification in the set of polyamino acid identifications and a polyamino acid intensity measured using the untargeted assay. In some cases, the polyamino acid intensity is measured using mass spectrometry. In some cases, the determining further comprises filtering the one or more base positions when a statistical significance value for the one or more base pair positions is less than a threshold statistical significance value. In some cases, the statistical significance value is a p-value. In some cases, the threshold statistical significance value is equal to, greater than, or less than 1e⁻², 1e⁻³, 1e⁻⁴, 1e⁻⁵, 1e⁻⁶, 1e⁻⁷, or 1e⁻⁸.

In some cases, the determining further comprises filtering the one or more base positions when a false discovery rate for the one or more base pair positions is less than a threshold false discovery rate. In some cases, the false discovery rate is determined by: (a) shuffling the proteomic data to generate a shuffled proteomic data; (b) identifying one or more decoy base positions in a shuffled proteomic data that covaries with at least one element in the proteomic information; and (c) normalizing the number of the one or more decoy base positions by the number of the one or more base positions. In some cases, the one or more decoy base positions may be identified in multiple runs. In some cases, the number of the one or more decoy base positions may be normalized by a mean number of decoy base positions identified in multiple runs.

In some cases, the method further comprises classifying the one or more base positions as a cis-pQTL or a trans-pQTL based on a distance between the one or more base positions and a gene that encodes a polyamino acid comprising the polyamino acid identification. In some cases, the one or more base positions are classified as a cis-pQTL when the distance is less than 1 megabases (Mbp) of a transcription start site of the gene. In some cases, the one or more base positions are classified as a cis-pQTL when the distance is less than 10, 9, 8, 7, 6, 5, 4, 3, 2, 1, 0.9, 0.8, 0.7, 0.6, 0.5, 0.4, 0.3, 0.2, or 0.1 megabases (Mbp) of a transcription start site of the gene. In some cases, the distance is greater than 5 kilobases (kb) upstream. In some cases, the distance is greater than 1, 2, 3, 4, 5, 6, 7, 8, 9, 10, 20, 30, 40, or 50 kb upstream. In some cases, the distance is less than 1 kb downstream. In some cases, the distance is less than 10, 9, 8, 7, 6, 5, 4, 3, 2, 1, 0.9, 0.8, 0.7, 0.6, 0.5, 0.4, 0.3, 0.2, or 0.1 kb downstream. Otherwise, a pQTL is considered to be a trans-pQTL. In some cases, the one or more regions in the one or more nucleic acid sequences comprises the gene that encodes a polyamino acid comprising the polyamino acid identification. In some cases, a pQTL may be a biomarker for a disease.

### Method for Identifying Similarly Expressed Proteoforms in Subject Groups

In some aspects, the present disclosure describes a method for assaying a biological sample. In some cases, the method comprises assaying a set of peptides from a plurality of biological samples to obtain a set of peptide identifications. In some cases, the method comprises identifying a set of protein groups based at least in part on the set of peptide identifications. In some cases, the method comprises determining, for a given protein group in the set of protein groups, a set of correlated peptides that are correlated in abundance across the plurality of biological samples. In some cases, the method comprises mapping the set of correlated peptides to a set of expressible proteoforms. In some cases, the method comprises identifying at least one proteoform common in the plurality of biological samples.

In some cases, the plurality of biological samples may comprise biological samples from a population of individuals. In some cases, the population of individual may comprise individuals afflicted or suspected of being afflicted with a disease. In some cases, the population of individual may comprise healthy individuals. In some cases, the population of individuals may comprise individuals at a certain stage of a disease. In some cases, the population of individuals may comprise males, females, age groups, or any combination thereof. In some cases, the population of individuals may comprise individuals with a similar diet.

In some cases, the set of correlated peptides may be associated with a characteristic of the plurality of biological samples. In some cases, the set of correlated peptides may be associated with a presence or an absence of a disease. In some cases, the set of correlated peptides may be associated with a severity of a disease. In some cases, the set of correlated peptides may be associated with a stage of a disease. In some cases, the set of correlated peptides may be associated with a likelihood of recovery by a patient. In some cases, the set of correlated peptides may be associated with a likelihood of success of a therapy or procedure on a patient. In some cases, the set of correlated peptides may be associated with a likelihood of a presence or an absence of a side-effect associated with a therapeutic.

In some cases, the proteoform may be associated with a characteristic of the plurality of biological samples. In some cases, the proteoform may be associated with a presence or an absence of a disease. In some cases, the proteoform may be associated with a severity of a disease. In some cases, the proteoform may be associated with a stage of a disease. In some cases, the proteoform may be associated with a likelihood of recovery by a patient. In some cases, the proteoform may be associated with a likelihood of success of a therapy or procedure on a patient. In some cases, the proteoform may be associated with a likelihood of a presence or an absence of a side-effect associated with a therapeutic.

In some cases, the set of peptides are peptide fragments derived from proteins expressed in the plurality of biological samples. In some cases, the set of peptides comprises a dynamic range of at least 5 orders of magnitude in the biological sample. In some cases, the set of peptides comprises a dynamic range of at least 6 orders of magnitude in the biological sample. In some cases, the set of peptides comprises a dynamic range of at least 7 orders of magnitude in the biological sample. In some cases, the set of peptides comprises a dynamic range of at least 8 orders of magnitude in the biological sample. In some cases, the set of peptides comprises a dynamic range of at least 9 orders of magnitude in the biological sample. In some cases, the set of peptides comprises a dynamic range of at least 10 orders of magnitude in the biological sample. In some cases, the set of peptides comprises a dynamic range of at least 11 orders of magnitude in the biological sample. In some cases, the set of peptides comprises a dynamic range of at least 12 orders of magnitude in the biological sample. In some cases, the set of peptides comprises a dynamic range of at least 5, 6, 7, 8, 9, 10, 11, or 12 orders of magnitude in the biological sample.

In some cases, the at least one untargeted assay comprises providing a plurality of surface regions comprising a plurality of surface types. In some cases, the at least one untargeted assay comprises contacting the biological sample with the plurality of surface regions to yield a set of adsorbed biomolecules on the plurality of surface regions. In some cases, the at least one untargeted assay comprises desorbing, from the plurality of surface regions, at least a portion of the set of adsorbed biomolecules to yield the set of polyamino acids. In some cases, the plurality of surface regions are disposed on a single continuous surface. In some cases, the plurality of surface regions are disposed on a plurality of discrete surfaces. In some cases, the plurality of discrete surfaces are surfaces of a plurality of particles.

In some cases, the plurality of surface types may comprise a surface that is configured to capture or interact with a biomolecule from a sample. For example, the surface may be functionalized with nucleic acid binding moieties, such as single stranded nucleic acids, which are capable of binding nucleic acids from a sample. In some aspects, the plurality of surface types may comprise a surface of a particle. In some aspects, the particle is a nanoparticle. In some aspects, the particle is a microparticle. In some aspects, the particle is a bead. In other cases, the particle is a synthesized particle. In some cases, a particle may be surface functionalized.

### Biological Sample

The present disclosure systems and methods for assaying a biological sample. In some cases, a biological sample may comprise a cell or be cell-free. In some cases, a biological sample may comprise a biofluid, such as blood, serum, plasma, urine, or cerebrospinal fluid (CSF). In some cases, a biofluid may be a fluidized solid, for example a tissue homogenate, or a fluid extracted from a biological sample. A biological sample may be, for example, a tissue sample or a fine needle aspiration (FNA) sample. A biological sample may be a cell culture sample. For example, a biofluid may be a fluidized cell culture extract. In some cases, a biological sample may be obtained from a subject. In some cases, the subject may be a human or a non-human. In some cases, the subject may be a plant, a fungus, or an archaeon. In some cases, a biological sample can contain a plurality of proteins or proteomic data, which may be analyzed after adsorption or binding of proteins to the surfaces of the various sensor element (e.g., particle) types in a panel and subsequent digestion of protein coronas.

In some cases, a biological sample may comprise plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof. In some cases, a biological sample may comprise multiple biological samples (e.g., pooled plasma from multiple subjects, or multiple tissue samples from a single subject). In some cases, a biological sample may comprise a single type of biofluid or biomaterial from a single source.

In some cases, a biological sample may be diluted or pre-treated. In some cases, a biological sample may undergo depletion (e.g., the biological sample comprises serum) prior to or following contact with a surface disclosed herein. In some cases, a biological sample may undergo physical (e.g., homogenization or sonication) or chemical treatment prior to or following contact with a surface disclosed herein. In some cases, a biological sample may be diluted prior to or following contact with a surface disclosed herein. In some cases, a dilution medium may comprise buffer or salts, or be purified water (e.g., distilled water). In some cases, a biological sample may be provided in a plurality partitions, wherein each partition may undergo different degrees of dilution. In some cases, a biological sample may comprise may undergo at least about 1.1-fold, 1.2-fold, 1.3-fold, 1.4-fold, 1.5-fold, 2-fold, 3-fold, 4-fold, 5-fold, 6-fold, 8-fold, 10-fold, 12-fold, 15-fold, 20-fold, 30-fold, 40-fold, 50-fold, 75-fold, 100-fold, 200-fold, 500-fold, or 1000-fold dilution.

In some cases, the biological sample may comprise a plurality of biomolecules. In some cases, a plurality of biomolecules may comprise polyamino acids. In some cases, the polyamino acids comprise peptides, proteins, or a combination thereof. In some cases, the plurality of biomolecules may comprise nucleic acids, carbohydrates, polyamino acids, or any combination thereof. A biological sample may comprise a member of any class of biomolecules, where “classes” may refer to any named category that defines a group of biomolecules having a common characteristic (e.g., proteins, nucleic acids, carbohydrates).

### Proteomic Analysis

As used herein, “proteomic analysis”, “protein analysis”, and the like, may refer to any system or method for analyzing proteins in a sample, including the systems and methods disclosed herein. The present disclosure systems and methods for assaying using one or more surface. In some cases, a surface may comprise a surface of a high surface-area material, such as nanoparticles, particles, or porous materials. As used herein, a “surface” may refer to a surface for assaying polyamino acids. When a particle composition, physical property, or use thereof is described herein, it shall be understood that a surface of the particle may comprise the same composition, the same physical property, or the same use thereof, in some cases. Similarly, when a surface composition, physical property, or use thereof is described herein, it shall be understood that a particle may comprise the surface to comprise the same composition, the same physical property, or the same use thereof.

Materials for particles and surfaces may include metals, polymers, magnetic materials, and lipids. In some cases, magnetic particles may be iron oxide particles. Examples of metallic materials include any one of or any combination of gold, silver, copper, nickel, cobalt, palladium, platinum, iridium, osmium, rhodium, ruthenium, rhenium, vanadium, chromium, manganese, niobium, molybdenum, tungsten, tantalum, iron, cadmium, or any alloys thereof. In some cases, a particle disclosed herein may be a magnetic particle, such as a superparamagnetic iron oxide nanoparticle (SPION). In some cases, a magnetic particle may be a ferromagnetic particle, a ferrimagnetic particle, a paramagnetic particle, a superparamagnetic particle, or any combination thereof (e.g., a particle may comprise a ferromagnetic material and a ferrimagnetic material).

The present disclosure describes panels of particles or surfaces. In some cases, a panel may comprise more than one distinct surface types. Panels described herein can vary in the number of surface types and the diversity of surface types in a single panel. For example, surfaces in a panel may vary based on size, polydispersity, shape and morphology, surface charge, surface chemistry and functionalization, and base material. In some cases, panels may be incubated with a sample to be analyzed for polyamino acids, polyamino acid concentrations, nucleic acids, nucleic acid concentrations, or any combination thereof. In some cases, polyamino acids in the sample adsorb to distinct surfaces to form one or more adsorption layers of biomolecules. The identity of the biomolecules and concentrations thereof in the one or more adsorption layers may depend on the physical properties of the distinct surfaces and the physical properties of the biomolecules. Thus, each surface type in a panel may have differently adsorbed biomolecules due to adsorbing a different set of biomolecules, different concentrations of a particular biomolecules, or a combination thereof. Each surface type in a panel may have mutually exclusive adsorbed biomolecules or may have overlapping adsorbed biomolecules.

In some cases, panels disclosed herein can be used to identify the number of distinct biomolecules disclosed herein over a wide dynamic range in a given biological sample. For example, a panel may enrich a subset of biomolecules in a sample, which can be identified over a wide dynamic range at which the biomolecules are present in a sample (e.g., a plasma sample). In some cases, the enriching may be selective—e.g., biomolecules in the subset may be enriched but biomolecules outside of the subset may not enriched and/or be depleted. In some cases, the subset may comprise proteins having different post-translational modifications. For example, a first particle type in the particle panel may enrich a protein or protein group having a first post-translational modification, a second particle type in the particle panel may enrich the same protein or same protein group having a second post-translational modification, and a third particle type in the particle panel may enrich the same protein or same protein group lacking a post-translational modification. In some cases, the panel including any number of distinct particle types disclosed herein, enriches and identifies a single protein or protein group by binding different domains, sequences, or epitopes of the protein or protein group. For example, a first particle type in the particle panel may enrich a protein or protein group by binding to a first domain of the protein or protein group, and a second particle type in the particle panel may enrich the same protein or same protein group by binding to a second domain of the protein or protein group. In some cases, a panel including any number of distinct particle types disclosed herein, may enrich and identify biomolecules over a dynamic range of at least 5, 6, 7, 8, 9, 10, 15, or 20 magnitudes. In some cases, a panel including any number of distinct particle types disclosed herein, may enrich and identify biomolecules over a dynamic range of at most 5, 6, 7, 8, 9, 10, 15, or 20 magnitudes.

A panel can have more than one surface type. Increasing the number of surface types in a panel can be a method for increasing the number of proteins that can be identified in a given sample.

A particle or surface may comprise a polymer. The polymer may constitute a core material (e.g., the core of a particle may comprise a particle), a layer (e.g., a particle may comprise a layer of a polymer disposed between its core and its shell), a shell material (e.g., the surface of the particle may be coated with a polymer), or any combination thereof. Examples of polymers include any one of or any combination of polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, a polyalkylene glycol (e.g., polyethylene glycol (PEG)), a polyester (e.g., poly(lactide-co-glycolide) (PLGA), polylactic acid, or polycaprolactone), or a copolymer of two or more polymers, such as a copolymer of a polyalkylene glycol (e.g., PEG) and a polyester (e.g., PLGA). The polymer may comprise a cross link. A plurality of polymers in a particle may be phase separated or may comprise a degree of phase separation.

Examples of lipids that can be used to form the particles or surfaces of the present disclosure include cationic, anionic, and neutrally charged lipids. For example, particles and/or surfaces can be made of any one of or any combination of dioleoylphosphatidylglycerol (DOPG), diacylphosphatidylcholine, diacylphosphatidylethanolamine, ceramide, sphingomyelin, cephalin, cholesterol, cerebrosides and diacylglycerols, dioleoylphosphatidylcholine (DOPC), dimyristoylphosphatidylcholine (DMPC), and dioleoylphosphatidylserine (DOPS), phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), lecithin, lysolecithin, phosphatidylethanolamine, lysophosphatidylethanolamine, dioleoylphosphatidylethanolamine (DOPE), dipalmitoyl phosphatidyl ethanolamine (DPPE), dimyristoylphosphoethanolamine (DMPE), distearoyl-phosphatidyl-ethanolamine (DSPE), palmitoyloleoyl-phosphatidylethanolamine (POPE) palmitoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleyolphosphatidylglycerol (POPG), 16-O-monomethyl PE, 16-O-dimethyl PE, 18-1-trans PE, palmitoyloleoyl-phosphatidylethanolamine (POPE), 1-stearoyl-2-oleoyl-phosphatidyethanolamine (SOPE), phosphatidylserine, phosphatidylinositol, sphingomyelin, cephalin, cardiolipin, phosphatidic acid, cerebrosides, dicetylphosphate, cholesterol, and any combination thereof.

A particle panel may comprise a combination of particles with silica and polymer surfaces. For example, a particle panel may comprise a SPION coated with a thin layer of silica, a SPION coated with poly(dimethyl aminopropyl methacrylamide) (PDMAPMA), and a SPION coated with poly(ethylene glycol) (PEG). A particle panel consistent with the present disclosure could also comprise two or more particles selected from the group consisting of silica coated SPION, an N-(3-Trimethoxysilylpropyl) diethylenetriamine coated SPION, a PDMAPMA coated SPION, a carboxyl-functionalized polyacrylic acid coated SPION, an amino surface functionalized SPION, a polystyrene carboxyl functionalized SPION, a silica particle, and a dextran coated SPION. A particle panel consistent with the present disclosure may also comprise two or more particles selected from the group consisting of a surfactant free carboxylate microparticle, a carboxyl functionalized polystyrene particle, a silica coated particle, a silica particle, a dextran coated particle, an oleic acid coated particle, a boronated nanopowder coated particle, a PDMAPMA coated particle, a Poly(glycidyl methacrylate-benzylamine) coated particle, and a Poly(N-[3-(Dimethylamino)propyl]methacrylamide-co-[2-(methacryloyloxy)ethyl]dimethyl-(3-sulfopropyl)ammonium hydroxide, P(DMAPMA-co-SBMA) coated particle. A particle panel consistent with the present disclosure may comprise silica-coated particles, N-(3-Trimethoxysilylpropyl)diethylenetriamine coated particles, poly(N-(3-(dimethylamino)propyl) methacrylamide) (PDMAPMA)-coated particles, phosphate-sugar functionalized polystyrene particles, amine functionalized polystyrene particles, polystyrene carboxyl functionalized particles, ubiquitin functionalized polystyrene particles, dextran coated particles, or any combination thereof.

A particle panel consistent with the present disclosure may comprise a silica functionalized particle, an amine functionalized particle, a silicon alkoxide functionalized particle, a carboxylate functionalized particle, and a benzyl or phenyl functionalized particle. A particle panel consistent with the present disclosure may comprise a silica functionalized particle, an amine functionalized particle, a silicon alkoxide functionalized particle, a polystyrene functionalized particle, and a saccharide functionalized particle. A particle panel consistent with the present disclosure may comprise a silica functionalized particle, an N-(3-Trimethoxysilylpropyl)diethylenetriamine functionalized particle, a PDMAPMA functionalized particle, a dextran functionalized particle, and a polystyrene carboxyl functionalized particle. A particle panel consistent with the present disclosure may comprise 5 particles including a silica functionalized particle, an amine functionalized particle, a silicon alkoxide functionalized particle.

Distinct surfaces or distinct particles of the present disclosure may differ by one or more physicochemical property. The one or more physicochemical property is selected from the group consisting of: composition, size, surface charge, hydrophobicity, hydrophilicity, roughness, density surface functionalization, surface topography, surface curvature, porosity, core material, shell material, shape, and any combination thereof. The surface functionalization may comprise a macromolecular functionalization, a small molecule functionalization, or any combination thereof. A small molecule functionalization may comprise an aminopropyl functionalization, amine functionalization, boronic acid functionalization, carboxylic acid functionalization, alkyl group functionalization, N-succinimidyl ester functionalization, monosaccharide functionalization, phosphate sugar functionalization, sulfurylated sugar functionalization, ethylene glycol functionalization, streptavidin functionalization, methyl ether functionalization, trimethoxysilylpropyl functionalization, silica functionalization, triethoxylpropylaminosilane functionalization, thiol functionalization, PCP functionalization, citrate functionalization, lipoic acid functionalization, ethyleneimine functionalization. A particle panel may comprise a plurality of particles with a plurality of small molecule functionalizations selected from the group consisting of silica functionalization, trimethoxysilylpropyl functionalization, dimethylamino propyl functionalization, phosphate sugar functionalization, amine functionalization, and carboxyl functionalization.

A small molecule functionalization may comprise a polar functional group. Non-limiting examples of polar functional groups comprise carboxyl group, a hydroxyl group, a thiol group, a cyano group, a nitro group, an ammonium group, an imidazolium group, a sulfonium group, a pyridinium group, a pyrrolidinium group, a phosphonium group or any combination thereof. In some embodiments, the functional group is an acidic functional group (e.g., sulfonic acid group, carboxyl group, and the like), a basic functional group (e.g., amino group, cyclic secondary amino group (such as pyrrolidyl group and piperidyl group), pyridyl group, imidazole group, guanidine group, etc.), a carbamoyl group, a hydroxyl group, an aldehyde group and the like.

A small molecule functionalization may comprise an ionic or ionizable functional group. Non-limiting examples of ionic or ionizable functional groups comprise an ammonium group, an imidazolium group, a sulfonium group, a pyridinium group, a pyrrolidinium group, a phosphonium group. A small molecule functionalization may comprise a polymerizable functional group. Non-limiting examples of the polymerizable functional group include a vinyl group and a (meth)acrylic group. In some embodiments, the functional group is pyrrolidyl acrylate, acrylic acid, methacrylic acid, acrylamide, 2-(dimethylamino)ethyl methacrylate, hydroxyethyl methacrylate and the like.

A surface functionalization may comprise a charge. For example, a particle can be functionalized to carry a net neutral surface charge, a net positive surface charge, a net negative surface charge, or a zwitterionic surface. Surface charge can be a determinant of the types of biomolecules collected on a particle. Accordingly, optimizing a particle panel may comprise selecting particles with different surface charges, which may not only increase the number of different proteins collected on a particle panel, but also increase the likelihood of identifying a biological state of a sample. A particle panel may comprise a positively charged particle and a negatively charged particle. A particle panel may comprise a positively charged particle and a neutral particle. A particle panel may comprise a positively charged particle and a zwitterionic particle. A particle panel may comprise a neutral particle and a negatively charged particle. A particle panel may comprise a neutral particle and a zwitterionic particle. A particle panel may comprise a negative particle and a zwitterionic particle. A particle panel may comprise a positively charged particle, a negatively charged particle, and a neutral particle. A particle panel may comprise a positively charged particle, a negatively charged particle, and a zwitterionic particle. A particle panel may comprise a positively charged particle, a neutral particle, and a zwitterionic particle. A particle panel may comprise a negatively charged particle, a neutral particle, and a zwitterionic particle.

A particle may comprise a single surface such as a specific small molecule, or a plurality of surface functionalizations, such as a plurality of different small molecules. Surface functionalization can influence the composition of a particle's biomolecule corona. Such surface functionalization can include small molecule functionalization or macromolecular functionalization. A surface functionalization may be coupled to a particle material such as a polymer, metal, metal oxide, inorganic oxide (e.g., silicon dioxide), or another surface functionalization.

A surface functionalization may comprise a small molecule functionalization, a macromolecular functionalization, or a combination of two or more such functionalizations. In some cases, a macromolecular functionalization may comprise a biomacromolecule, such as a protein or a polynucleotide (e.g., a 100-mer DNA molecule). A macromolecular functionalization may comprise a protein, polynucleotide, or polysaccharide, or may be comparable in size to any of the aforementioned classes of species. In some cases, A surface functionalization may comprise an ionizable moiety. In some cases, a surface functionalization may comprise pKa of at least about 1, 2, 3, 4, 5, 6, 7, 8, 9, 10, 11, 12, 13, or 14. In some cases, a surface functionalization may comprise pKa of at most about 1, 2, 3, 4, 5, 6, 7, 8, 9, 10, 11, 12, 13, or 14. In some cases, a small molecule functionalization may comprise a small organic molecule such as an alcohol (e.g., octanol), an amine, an alkane, an alkene, an alkyne, a heterocycle (e.g., a piperidinyl group), a heteroaromatic group, a thiol, a carboxylate, a carbonyl, an amide, an ester, a thioester, a carbonate, a thiocarbonate, a carbamate, a thiocarbamate, a urea, a thiourea, a halogen, a sulfate, a phosphate, a monosaccharide, a disaccharide, a lipid, or any combination thereof. For example, a small molecule functionalization may comprise a phosphate sugar, a sugar acid, or a sulfurylated sugar.

In some cases, a macromolecular functionalization may comprise a specific form of attachment to a particle. In some cases, a macromolecule may be tethered to a particle via a linker. In some cases, the linker may hold the macromolecule close to the particle, thereby restricting its motion and reorientation relative to the particle, or may extend the macromolecule away from the particle. In some cases, the linker may be rigid (e.g., a polyolefin linker) or flexible (e.g., a nucleic acid linker). In some cases, a linker may be at least about 0.5, 1, 2, 3, 4, 5, 6, 7, 8, 9, 10, 15, 20, 25, or 30 nm in length. In some cases, a linker may be at most about 0.5, 1, 2, 3, 4, 5, 6, 7, 8, 9, 10, 15, 20, 25, or 30 nm in length. As such, a surface functionalization on a particle may project beyond a primary corona associated with the particle. In some cases, a surface functionalization may also be situated beneath or within a biomolecule corona that forms on the particle surface. In some cases, a macromolecule may be tethered at a specific location, such as at a protein's C-terminus, or may be tethered at a number of possible sites. For example, a peptide may be covalent attached to a particle via any of its surface exposed lysine residues.

In some cases, a particle may be contacted with a biological sample (e.g., a biofluid) to form a biomolecule corona. In some cases, a biomolecule corona may comprise at least two biomolecules that do not share a common binding motif. The particle and biomolecule corona may be separated from the biological sample, for example by centrifugation, magnetic separation, filtration, or gravitational separation. The particle types and biomolecule corona may be separated from the biological sample using a number of separation techniques. Non-limiting examples of separation techniques include comprises magnetic separation, column-based separation, filtration, spin column-based separation, centrifugation, ultracentrifugation, density or gradient-based centrifugation, gravitational separation, or any combination thereof. A protein corona analysis may be performed on the separated particle and biomolecule corona. A protein corona analysis may comprise identifying one or more proteins in the biomolecule corona, for example by mass spectrometry. In some cases, a single particle type may be contacted with a biological sample. In some cases, a plurality of particle types may be contacted to a biological sample. In some cases, the plurality of particle types may be combined and contacted to the biological sample in a single sample volume. In some cases, the plurality of particle types may be sequentially contacted to a biological sample and separated from the biological sample prior to contacting a subsequent particle type to the biological sample. In some cases, adsorbed biomolecules on the particle may have compressed (e.g., smaller) dynamic range compared to a given original biological sample.

In some cases, the particles of the present disclosure may be used to serially interrogate a sample by incubating a first particle type with the sample to form a biomolecule corona on the first particle type, separating the first particle type, incubating a second particle type with the sample to form a biomolecule corona on the second particle type, separating the second particle type, and repeating the interrogating (by incubation with the sample) and the separating for any number of particle types. In some cases, the biomolecule corona on each particle type used for serial interrogation of a sample may be analyzed by protein corona analysis. The biomolecule content of the supernatant may be analyzed following serial interrogation with one or more particle types.

In some cases, a method of the present disclosure may identify a large number of unique biomolecules (e.g., proteins) in a biological sample (e.g., a biofluid). In some cases, a surface disclosed herein may be incubated with a biological sample to adsorb at least 1000, 2000, 3000, 4000, 5000, 6000, 7000, 8000, 9000, or 10000 unique biomolecules. In some cases, a surface disclosed herein may be incubated with a biological sample to adsorb at most 1000, 2000, 3000, 4000, 5000, 6000, 7000, 8000, 9000, or 10000 unique biomolecules. In some cases, a surface disclosed herein may be incubated with a biological sample to adsorb at least 1000, 2000, 3000, 4000, 5000, 6000, 7000, 8000, 9000, or 10000 unique biomolecule groups. In some cases, a surface disclosed herein may be incubated with a biological sample to adsorb at most 1000, 2000, 3000, 4000, 5000, 6000, 7000, 8000, 9000, or 10000 unique biomolecule groups. In some cases, several different types of surfaces can be used, separately or in combination, to identify large numbers of proteins in a particular biological sample. In other words, surfaces can be multiplexed in order to bind and identify large numbers of biomolecules in a biological sample.

In some cases, a method of the present disclosure may identify a large number of unique proteoforms in a biological sample. In some cases, a method may identify at least about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 unique proteoforms. In some cases, a method may identify at most about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 unique proteoforms. In some cases, a surface disclosed herein may be incubated with a biological sample to adsorb at least 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 unique proteoforms. In some cases, a surface disclosed herein may be incubated with a biological sample to adsorb at most 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 unique proteoforms. In some cases, several different types of surfaces can be used, separately or in combination, to identify large numbers of proteins in a particular biological sample. In other words, surfaces can be multiplexed in order to bind and identify large numbers of biomolecules in a biological sample.

Biomolecules collected on particles may be subjected to further analysis. In some cases, a method may comprise collecting a biomolecule corona or a subset of biomolecules from a biomolecule corona. In some cases, the collected biomolecule corona or the collected subset of biomolecules from the biomolecule corona may be subjected to further particle-based analysis (e.g., particle adsorption). In some cases, the collected biomolecule corona or the collected subset of biomolecules from the biomolecule corona may be purified or fractionated (e.g., by a chromatographic method). In some cases, the collected biomolecule corona or the collected subset of biomolecules from the biomolecule corona may be analyzed (e.g., by mass spectrometry).

In some cases, the panels disclosed herein can be used to identify a number of proteins, peptides, protein groups, or protein classes using a protein analysis workflow described herein (e.g., a protein corona analysis workflow). In some cases, protein analysis may comprise contacting a sample to distinct surface types (e.g., a particle panel), forming adsorbed biomolecule layers on the distinct surface types, and identifying the biomolecules in the adsorbed biomolecule layers (e.g., by mass spectrometry). Feature intensities, as disclosed herein, may refer to the intensity of a discrete spike (“feature”) seen on a plot of mass to charge ratio versus intensity from a mass spectrometry run of a sample. In some cases, these features can correspond to variably ionized fragments of peptides and/or proteins. In some cases, using the data analysis methods described herein, feature intensities can be sorted into protein groups. In some cases, protein groups may refer to two or more proteins that are identified by a shared peptide sequence. In some cases, a protein group can refer to one protein that is identified using a unique identifying sequence. For example, if in a sample, a peptide sequence is assayed that is shared between two proteins (Protein 1: XYZZX and Protein 2: XYZYZ), a protein group could be the “XYZ protein group” having two members (protein 1 and protein 2). In some cases, if the peptide sequence is unique to a single protein (Protein 1), a protein group could be the “ZZX” protein group having one member (Protein 1). In some cases, each protein group can be supported by more than one peptide sequence. In some cases, protein detected or identified according to the instant disclosure can refer to a distinct protein detected in the sample (e.g., distinct relative other proteins detected using mass spectrometry). In some cases, analysis of proteins present in distinct coronas corresponding to the distinct surface types in a panel yields a high number of feature intensities. In some cases, this number decreases as feature intensities are processed into distinct peptides, further decreases as distinct peptides are processed into distinct proteins, and further decreases as peptides are grouped into protein groups (two or more proteins that share a distinct peptide sequence).

In some cases, the methods disclosed herein include isolating one or more particle types from a sample or from more than one sample (e.g., a biological sample or a serially interrogated sample). The particle types can be rapidly isolated or separated from the sample using a magnet. Moreover, multiple samples that are spatially isolated can be processed in parallel. In some cases, the methods disclosed herein provide for isolating or separating a particle type from unbound protein in a sample. In some cases, a particle type may be separated by a variety of means, including but not limited to magnetic separation, centrifugation, filtration, or gravitational separation. In some cases, particle panels may be incubated with a plurality of spatially isolated samples, wherein each spatially isolated sample is in a well in a well plate (e.g., a 96-well plate). In some cases, the particle in each of the wells of the well plate can be separated from unbound protein present in the spatially isolated samples by placing the entire plate on a magnet. In some cases, this simultaneously pulls down the superparamagnetic particles in the particle panel. In some cases, the supernatant in each sample can be removed to remove the unbound protein. In some cases, these steps (incubate, pull down) can be repeated to effectively wash the particles, thus removing residual background unbound protein that may be present in a sample.

In some cases, the systems and methods disclosed herein may also elucidate protein classes or interactions of the protein classes. In some cases, a protein class may comprise a set of proteins that share a common function (e.g., amine oxidases or proteins involved in angiogenesis); proteins that share common physiological, cellular, or subcellular localization (e.g., peroxisomal proteins or membrane proteins); proteins that share a common cofactor (e.g., heme or flavin proteins); proteins that correspond to a particular biological state (e.g., hypoxia related proteins); proteins containing a particular structural motif (e.g., a cupin fold); proteins that are functionally related (e.g., part of a same metabolic pathway); or proteins bearing a post-translational modification (e.g., ubiquitinated or citrullinated proteins). In some cases, a protein class may contain at least 2 proteins, 5 proteins, 10 proteins, 20 proteins, 40 proteins, 60 proteins, 80 proteins, 100 proteins, 150 proteins, 200 proteins, or more.

In some cases, the proteomic data of the biological sample can be identified, measured, and quantified using a number of different analytical techniques. For example, proteomic data can be generated using SDS-PAGE or any gel-based separation technique. In some cases, peptides and proteins can also be identified, measured, and quantified using an immunoassay, such as ELISA. In some cases, proteomic data can be identified, measured, and quantified using mass spectrometry, high performance liquid chromatography, LC-MS/MS, Edman Degradation, immunoaffinity techniques, and other protein separation techniques.

In some cases, an assay may comprise protein collection of particles, protein digestion, and mass spectrometric analysis (e.g., MS, LC-MS, LC-MS/MS). In some cases, the digestion may comprise chemical digestion, such as by cyanogen bromide or 2-Nitro-5-thiocyanatobenzoic acid (NTCB). In some cases, the digestion may comprise enzymatic digestion, such as by trypsin or pepsin. In some cases, the digestion may comprise enzymatic digestion by a plurality of proteases. In some cases, the digestion may comprise a protease selected from among the group consisting of trypsin, chymotrypsin, Glu C, Lys C, elastase, subtilisin, proteinase K, thrombin, factor X, Arg C, papaine, Asp N, thermolysine, pepsin, aspartyl protease, cathepsin D, zinc mealloprotease, glycoprotein endopeptidase, proline, aminopeptidase, prenyl protease, caspase, kex2 endoprotease, or any combination thereof. In some cases, the digestion may cleave peptides at random positions. In some cases, the digestion may cleave peptides at a specific position (e.g., at methionines) or sequence (e.g., glutamate-histidine-glutamate). In some cases, the digestion may enable similar proteins to be distinguished. For example, an assay may resolve 8 distinct proteins as a single protein group with a first digestion method, and as 8 separate proteins with distinct signals with a second digestion method. In some cases, the digestion may generate an average peptide fragment length of 8 to 15 amino acids. In some cases, the digestion may generate an average peptide fragment length of 12 to 18 amino acids. In some cases, the digestion may generate an average peptide fragment length of 15 to 25 amino acids. In some cases, the digestion may generate an average peptide fragment length of 20 to 30 amino acids. In some cases, the digestion may generate an average peptide fragment length of 30 to 50 amino acids.

In some cases, an assay may rapidly generate and analyze proteomic data. In some cases, beginning with an input biological sample (e.g., a buccal or nasal smear, plasma, or tissue), a method of the present disclosure may generate and analyze proteomic data in less than about 1, 2, 3, 4, 5, 6, 7, 8, 12, 16, 20, 24, or 48 hours. In some cases, the analyzing may comprise identifying a protein group. In some cases, the analyzing may comprise identifying a protein class. In some cases, the analyzing may comprise quantifying an abundance of a biomolecule, a peptide, a protein, protein group, or a protein class. In some cases, the analyzing may comprise identifying a ratio of abundances of two biomolecules, peptides, proteins, protein groups, or protein classes. In some cases, the analyzing may comprise identifying a biological state.

An example of a particle type of the present disclosure may be a carboxylate (Citrate) superparamagnetic iron oxide nanoparticle (SPION), a phenol-formaldehyde coated SPION, a silica-coated SPION, a polystyrene coated SPION, a carboxylated poly(styrene-co-methacrylic acid) coated SPION, a N-(3-Trimethoxysilylpropyl)diethylenetriamine coated SPION, a poly(N-(3-(dimethylamino)propyl) methacrylamide) (PDMAPMA)-coated SPION, a 1,2,4,5-Benzenetetracarboxylic acid coated SPION, a poly(Vinylbenzyltrimethylammonium chloride) (PVBTMAC) coated SPION, a carboxylate, PAA coated SPION, a poly(oligo(ethylene glycol) methyl ether methacrylate) (POEGMA)-coated SPION, a carboxylate microparticle, a polystyrene carboxyl functionalized particle, a carboxylic acid coated particle, a silica particle, a carboxylic acid particle of about 150 nm in diameter, an amino surface microparticle of about 0.4-0.6 μm in diameter, a silica amino functionalized microparticle of about 0.1-0.39 μm in diameter, a Jeffamine surface particle of about 0.1-0.39 μm in diameter, a polystyrene microparticle of about 2.0-2.9 μm in diameter, a silica particle, a carboxylated particle with an original coating of about 50 nm in diameter, a particle coated with a dextran based coating of about 0.13 μm in diameter, or a silica silanol coated particle with low acidity. In some cases, a particle may lack functionalized specific binding moieties for specific binding on its surface. In some cases, a particle may lack functionalized proteins for specific binding on its surface. In some cases, a surface functionalized particle does not comprise an antibody or a T cell receptor, a chimeric antigen receptor, a receptor protein, or a variant or fragment thereof. In some cases, the ratio between surface area and mass can be a determinant of a particle's properties. The particles disclosed herein can have surface area to mass ratios of 3 to 30 cm²/mg, 5 to 50 cm²/mg, 10 to 60 cm²/mg, 15 to 70 cm²/mg, 20 to 80 cm²/mg, 30 to 100 cm²/mg, 35 to 120 cm²/mg, 40 to 130 cm²/mg, 45 to 150 cm²/mg, 50 to 160 cm²/mg, 60 to 180 cm²/mg, 70 to 200 cm²/mg, 80 to 220 cm²/mg, 90 to 240 cm²/mg, 100 to 270 cm²/mg, 120 to 300 cm²/mg, 200 to 500 cm²/mg, 10 to 300 cm²/mg, 1 to 3000 cm²/mg, 20 to 150 cm²/mg, 25 to 120 cm²/mg, or from 40 to 85 cm²/mg. Small particles (e.g., with diameters of 50 nm or less) can have significantly higher surface area to mass ratios, stemming in part from the higher order dependence on diameter by mass than by surface area. In some cases (e.g., for small particles), the particles can have surface area to mass ratios of 200 to 1000 cm²/mg, 500 to 2000 cm²/mg, 1000 to 4000 cm²/mg, 2000 to 8000 cm²/mg, or 4000 to 10000 cm²/mg. In some cases (e.g., for large particles), the particles can have surface area to mass ratios of 1 to 3 cm²/mg, 0.5 to 2 cm²/mg, 0.25 to 1.5 cm²/mg, or 0.1 to 1 cm²/mg. A particle may comprise a wide array of physical properties. A physical property of a particle may include composition, size, surface charge, hydrophobicity, hydrophilicity, amphipathicity, surface functionality, surface topography, surface curvature, porosity, core material, shell material, shape, zeta potential, and any combination thereof. A particle may have a core-shell structure. In some cases, a core material may comprise metals, polymers, magnetic materials, paramagnetic materials, oxides, and/or lipids. In some cases, a shell material may comprise metals, polymers, magnetic materials, oxides, and/or lipids.

### Proteomic Information

In some cases, proteomic information or data can refer to information about substances comprising a peptide and/or a protein component. In some cases, proteomic information may comprise primary structure information, secondary structure information, tertiary structure information, or quaternary information about the peptide or a protein. In some cases, proteomic information may comprise information about protein-ligand interactions, wherein a ligand may comprise any one of various biological molecules and substances that may be found in living organisms, such as, nucleotides, nucleic acids, amino acids, peptides, proteins, monosaccharides, polysaccharides, lipids, phospholipids, hormones, or any combination thereof.

In some cases, proteomic information may comprise information about a single cell, a tissue, an organ, a system of tissues and/or organs (such as cardiovascular, respiratory, digestive, or nervous systems), or an entire multicellular organism. In some cases, proteomic information may comprise information about an individual (e.g., an individual human being or an individual bacterium), or a population of individuals (e.g., human beings with diagnosed with cancer or a colony of bacteria). Proteomic information may comprise information from various forms of life, including forms of life from the Archaea, the Bacteria, the Eukarya, the Protozoa, the Chromista, the Plantae, the Fungi, or from the Animalia. In some cases, proteomic information may comprise information from viruses.

In some cases, proteomic information may comprise information relating exons and introns in the code of life. In some cases, proteomic information may comprise information regarding variations in the primary structure, variations in the secondary structure, variations in the tertiary structure, or variations in the quaternary structure of peptides and/or proteins. In some cases, proteomic information may comprise information regarding variations in the expression of exons, including alternative splicing variations, structural variations, or both. In some cases, proteomic information may comprise conformation information, post-translational modification information, chemical modification information (e.g., phosphorylation), cofactor (e.g., salts or other regulatory chemicals) association information, or substrate association information of peptides and/or proteins.

In some cases, proteomic information may comprise information related to various proteoforms in a sample. In some cases, a proteomic information may comprise information related to peptide variants, protein variants, or both. In some cases, a proteomic information may comprise information related to splicing variants, allelic variants, post-translation modification variants, or any combination thereof. In some cases, peptide variants or protein variants may comprise a post-translation modification. In some cases, the post-translational modification comprises acylation, alkylation, prenylation, flavination, amination, deamination, carboxylation, decarboxylation, nitrosylation, halogenation, sulfurylation, glutathionylation, oxidation, oxygenation, reduction, ubiquitination, SUMOylation, neddylation, myristoylation, palmitoylation, isoprenylation, farnesylation, geranylgeranylation, glypiation, glycosylphosphatidylinositol anchor formation, lipoylation, heme functionalization, phosphorylation, phosphopantetheinylation, retinylidene Schiff base formation, diphthamide formation, ethanolamine phosphoglycerol functionalization, hypusine formation, beta-Lysine addition, acetylation, formylation, methylation, amidation, amide bond formation, butyrylation, gamma-carboxylation, glycosylation, polysialylation, malonylation, hydroxylation, iodination, nucleotide addition, phosphate ester formation, phosphoramidate formation, adenylation, uridylylation, propionylation, pyroglutamate formation, gluthathionylation, sulfenylation, sulfinylation, sulfonylation, succinylation, sulfation, glycation, carbonylation, isopeptide bond formation, biotinylation, carbamylation, oxidation, pegylation, citrullination, deamidation, eliminylation, disulfide bond formation, proteolytic cleavage, isoaspartate formation, racemization, protein splicing, chaperon-assisted folding, or any combination thereof.

### Genomic Analysis

As used herein, “genomic analysis”, “nucleic acid analysis”, and the like, may refer to any system or method for analyzing proteins in a sample, including the systems and methods disclosed herein. The present disclosure describes various compositions and methods for analyzing (e.g., detecting or sequencing) nucleic acids. In some cases, genotypic (or genomic) information may be obtained using some of the compositions and methods of the present disclosure. In some cases, genotypic information can refer to information about substances comprising a nucleotide and/or a nucleic acid component. In some cases, genotypic information may comprise epigenetic information. In some cases, epigenetic information may comprise histone modification, DNA methylation, accessibility of different regions in a genome, dynamics changes thereof, or any combination thereof. In some cases, genotypic information may comprise primary structure information, secondary structure information, tertiary structure information, or quaternary information about a nucleic acid. In some cases, genotypic information may comprise information about nucleic acid-ligand interactions, wherein a ligand may comprise any one of various biological molecules and substances that may be found in living organisms, such as, nucleotides, nucleic acids, amino acids, peptides, proteins, monosaccharides, polysaccharides, lipids, phospholipids, hormones, or any combination thereof. In some cases, genotypic information may comprise a rate or prevalence of apoptosis of a healthy cell or a diseased cell. In some cases, genotypic information may comprise a state of a cell, such as a healthy state or a diseased state. In some cases, genotypic information may comprise chemical modification information of a nucleic acid molecule. In some cases, a chemical modification may comprise methylation, demethylation, amination, deamination, acetylation, oxidation, oxygenation, reduction, or any combination thereof. In some cases, genotypic information may comprise information regarding from which type of cell a biological sample originates. In some cases, genotypic information may comprise information about an untranslated region of nucleic acids.

In some cases, genotypic information may comprise information about a single cell, a tissue, an organ, a system of tissues and/or organs (such as cardiovascular, respiratory, digestive, or nervous systems), or an entire multicellular organism. In some cases, genotypic information may comprise information about an individual (e.g., an individual human being or an individual bacterium), or a population of individuals (e.g., human beings with diagnosed with cancer or a colony of bacteria). Genotypic information may comprise information from various forms of life, including forms of life from the Archaea, the Bacteria, the Eukarya, the Protozoa, the Chromista, the Plantae, the Fungi, or from the Animalia. In some cases, genotypic information may comprise information from viruses.

In some cases, genotypic information may comprise information relating exons and introns in the code of life. In some cases, genotypic information may comprise information regarding variations or mutations in the primary structure of nucleic acids, including base substitutions, deletions, insertions, or any combination thereof. In some cases, genotypic information may comprise information regarding the inclusion of non-canonical nucleobases in nucleic acids. In some cases, genotypic information may comprise information regarding variations or mutations in epigenetics.

In some cases, genotypic information may comprise information regarding variations in the primary structure, variations in the secondary structure, variations in the tertiary structure, or variations in the quaternary structure of peptides and/or proteins that one or more nucleic acids encode.

In some cases, the set of nucleic acids comprise an exome of the biological sample. In some cases, the set of nucleic acids comprise a genome, an epigenome, a transcriptome, a metabolome, a secretome, or any combination thereof. In some cases, the set of nucleic acids comprises a portion of the exome of the biological sample. In some cases, the set of nucleic acids comprise a portion of the genome, an epigenome, a transcriptome, a metabolome, a secretome, or any combination thereof. In some cases, the genotypic information comprises an exome sequence of the biological sample. In some cases, the genotypic information comprises one or more sequences of the genome, an epigenome, a transcriptome, a metabolome, a secretome, or any combination thereof.

Various sequencing methods and various sequencing reagents may be used to obtain genotypic information. In some cases, the sequencing methods disclosed herein may comprise enriching one or more nucleic acid molecules from a sample. This may comprise enrichment in solution, enrichment on a sensor element (e.g., a particle), enrichment on a substrate (e.g., a surface of an Eppendorf tube), or selective removal of a nucleic acid (e.g., by sequence-specific affinity precipitation). Enrichment may comprise amplification, including differential amplification of two or more different target nucleic acids. Differential amplification may be based on sequence, CG-content, or post-transcriptional modifications, such as methylation state. In some cases, enrichment may comprise hybridization methods, such as pull-down methods. For example, a substrate partition may comprise immobilized nucleic acids capable of hybridizing to nucleic acids of a particular sequence, and thereby capable of isolating particular nucleic acids from a complex biological solution. In some cases, hybridization may target genes, exons, introns, regulatory regions, splice sites, reassembly genes, among other nucleic acid targets. In some cases, hybridization can utilize a pool of nucleic acid probes that are designed to target multiple distinct sequences, or to tile a single sequence.

Enrichment may comprise a hybridization reaction and may generate a subset of nucleic acid molecules from a biological sample. Hybridization may be performed in solution, on a substrate surface (e.g., a wall of a well in a microwell plate), on a sensor element, or any combination thereof. A hybridization method may be sensitive for single nucleotide polymorphisms. For example, a hybridization method may comprise molecular inversion probes.

Enrichment may also comprise amplification. Suitable amplification methods include polymerase chain reaction (PCR), solid-phase PCR, RT-PCR, qPCR, multiplex PCR, touchdown PCR, nanoPCR, nested PCR, hot start PCR, helicase-dependent amplification, loop mediated isothermal amplification (LAMP), self-sustained sequence replication, nucleic acid sequence based amplification, strand displacement amplification, rolling circle amplification, ligase chain reaction, and any other suitable amplification technique.

The sequencing may target a specific sequence or region of a genome. The sequencing may target a type of sequence, such as exons. In some cases, the sequencing comprises exome sequencing. In some cases, the sequencing comprises whole exome sequencing. The sequencing may target chromatinated or non-chromatinated nucleic acids. The sequencing may be sequence-non specific (e.g., provide a reading regardless of the target sequence). The sequencing may target a polymerase accessible region of the genome. The sequencing may target nucleic acids localized in a part of a cell, such as the mitochondria or the cytoplasm. The sequencing may target nucleic acids localized in a cell, tissue, or an organ. The sequencing may target RNA, DNA, any other nucleic acid, or any combination thereof.

‘Nucleic acid’ may refer to a polymeric form of nucleotides of any length, in single-, double- or multi-stranded form. A nucleic acid may comprise any combination of ribonucleotides, deoxyribonucleotides, and natural and non-natural analogues thereof, including 5-bromouracil, peptide nucleic acids, locked nucleotides, glycol nucleotides, threose nucleotides, dideoxynucleotides, 3′-deoxyribonucleotides, dideoxyribonucleotides, 7-deaza-GTP, fluorophores-bound nucleotides, thiol containing nucleotides, biotin linked nucleotides, methyl-7-guanosine, methylated nucleotides, inosine, thiouridine, pseudourdine, dihydrouridine, queuosine, and wyosine. A nucleic acid may comprise a gene, a portion of a gene, an exon, an intron, messenger RNA (mRNA), transfer RNA (tRNA), ribosomal RNA (rRNA), short interfering RNA (siRNA), short-hairpin RNA (shRNA), micro-RNA (miRNA), a ribozyme, cDNA, a recombinant nucleic acid, a branched nucleic acid, a plasmid, cell-free DNA (cfDNA), cell-free RNA (cfRNA), genomic DNA, mitochondrial DNA (mtDNA), circulating tumor DNA (ctDNA), long non-coding RNA, telomerase RNA, Piwi-interacting RNA, small nuclear RNA (snRNA), small interfering RNA, YRNA, circular RNA, small nucleolar RNA, or pseudogene RNA. A nucleic acid may comprise a DNA or RNA molecule. A nucleic acid may also have a defined 3-dimensional structure. In some cases, a nucleic acid may comprise a non-canonical nucleobase or a nucleotide, such as hypoxanthine, xanthine, 7-methylguanine, 5,6-dihydrouracil, 5-methylcytosine, or any combination thereof. Nucleic acids may also comprise non-nucleic acid molecules.

A nucleic acid may be derived from various sources. In some cases, a nucleic acid may be derived from an exosome, an apoptotic body, a tumor cell, a healthy cell, a virtosome, an extracellular membrane vesicle, a neutrophil extracellular trap (NET), or any combination thereof.

A nucleic acid may comprise various lengths. In some cases, a nucleic acid may comprise at least about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 nucleotides. In some cases, a nucleic acid may comprise at most about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 nucleotides.

Various reagents may be used for sequencing. In some cases, a reagent may comprise primers, oligonucleotides, switch oligonucleotides, adapters, amplification adapters, polymerases, dNTPs, co-factors, buffers, enzymes, ionic co-factors, ligase, reverse transcriptase, restriction enzymes, endonucleases, transposase, protease, proteinase K, DNase, RNase, lysis agents, lysozymes, achromopeptidase, lysostaphin, labiase, kitalase, lyticase, inhibitors, inactivating agents, chelating agents, EDTA, crowding agents, reducing agents, DTT, surfactants, TritonX-IOO, Tween 20, sodium dodecyl sulfate, sarcosyl, or any combination thereof.

Various methods for sequencing nucleic acids may be used. In some cases, sequencing may comprise sequencing a whole genome or portions thereof. Sequencing may comprise sequencing a whole genome, a whole exome, portions thereof (e.g., a panel of genes, including potentially coding and non-coding regions thereof). Sequencing may comprise sequencing a transcriptome or portion thereof. Sequencing may comprise sequencing an exome or portion thereof. Sequencing coverage may be optimized based on analytical or experimental setup, or desired sequencing footprint. In some cases, a nucleic acid sequencing method may comprise high-throughput sequencing, next-generation sequencing, flow sequencing, massively-parallel sequencing, shotgun sequencing, single-molecule real-time sequencing, ion semiconductor sequencing, electrophoretic sequencing, pyrosequencing, sequencing by synthesis, combinatorial probe anchor synthesis sequencing, sequencing by ligation, nanopore sequencing, GenapSys sequencing, chain termination sequencing, polony sequencing, 454 pyrosequencing, reversible terminated chemistry sequencing, heliscope single molecule sequencing, tunneling currents DNA sequencing, sequencing by hybridization, clonal single molecule array sequencing, sequencing with MS, DNA-seq, RNA-seq, ATAC-seq, methyl-seq, ChIP-seq, or any combination thereof. The sequencing methods of the present disclosure may involve sequence analysis of RNA. RNA sequences or expression levels may be analyzed by using a reverse transcription reaction to generate complementary DNA (cDNA) molecules from RNA for sequencing or by using reverse transcription polymerase chain reaction for quantification of expression levels. The sequencing methods of the present disclosure may detect RNA structural variants and isoforms, such as splicing variants and structural variants. The sequencing methods of the present disclosure may quantify RNA sequences or structural variants. In some cases, a sequencing may method comprise spatial sequencing, single-cell sequencing or any combination thereof.

In some cases, nucleic acids may be processed by standard molecular biology techniques for downstream applications. In some cases, nucleic acids may be prepared from nucleic acids isolated from a sample of the present disclosure. In some cases, the nucleic acids may subsequently be attached to an adaptor polynucleotide sequence, which may comprise a double stranded nucleic acid. In some cases, the nucleic acids may be end repaired prior to attaching to the adaptor polynucleotide sequences. In some cases, adaptor polynucleotides may be attached to one or both ends of the nucleotide sequences. In some cases, the same or different adaptor may be bound to each end of the fragment, thereby producing an “adaptor-nucleic acid-adaptor” construct. In some cases, a plurality of the same or different adaptor may be bound to each end of the fragment. In some cases, different adaptors may be attached to each end of the nucleic acid when adaptors are attached to both ends of the nucleic acid.

In some cases, an oligonucleotide tag complementary to a sequencing primer may be incorporated with adaptors attached to a target nucleic acid. For analysis of multiple samples, different oligonucleotide tags complementary to separate sequencing primers may be incorporated with adaptors attached to a target nucleic acid.

In some cases, an oligonucleotide index tag may also be incorporated with adaptors attached to a target nucleic acid. In cases in which deletion products are generated from a plurality of polynucleotides prior to hybridizing the deletion products to a nucleic acid immobilized on a structure (e.g., a sensor element such as a particle), polynucleotides corresponding to different nucleic acids of interest may first be attached to different oligonucleotide tags such that subsequently generated deletion products corresponding to different nucleic acids of interest may be grouped or differentiated. Consequently, deletion products derived from the same nucleic acid of interest may have the same oligonucleotide index tag such that the index tag identifies sequencing reads derived from the same nucleic acid of interest. Likewise, deletion products derived from different nucleic acids of interest may have different oligonucleotide index tags to allow them to be grouped or differentiated such as on a sensor element. Oligonucleotide index tags may range in length from about 5, 10, 15, 20, 25, 30, 35, 40, 45, 50, 75, to 100 nucleotides or base pairs, or any length in between.

In some cases, the oligonucleotide index tags may be added separately or in conjunction with a primer, primer binding site or other component. Conversely, a pair-end read may be performed, wherein the read from the first end may comprise a portion of the sequence of interest and the read from the other (second) end may be utilized as a tag to identify the fragment from which the first read originated.

In some cases, a sequencing read may be initiated from the point of incorporation of the modified nucleotide into an extended capture probe. In some cases, a sequencing primer may be hybridized to extended capture probes or their complements, which may be optionally amplified prior to initiating a sequence read, and extended in the presence of natural nucleotides. In some cases, extension of the sequencing primer may stall at the point of incorporation of the first modified nucleotide incorporated in the template, and a complementary modified nucleotide may be incorporated at the point of stall using a polymerase capable of incorporating a modified nucleotide (e.g. TiTaq polymerase). In some cases, a sequencing read may be initiated at the first base after the stall or point of modified nucleotide incorporation. In a sequencing-by-synthesis method, a sequencing read may be initiated at the first base after the stall or point of modified nucleotide incorporation.

The present disclosure describes methods and compositions related to nucleic acid (polynucleotide) sequencing. Some methods of the present disclosure may provide for identification and quantification of nucleic acids in a subject or a sample. In some cases, the nucleotide sequence of a portion of a target nucleic acid or fragment thereof may be determined using a variety of methods and devices. Examples of sequencing methods include electrophoretic, sequencing by synthesis, sequencing by ligation, sequencing by hybridization, single-molecule sequencing, and real time sequencing methods. In some cases, the process to determine the nucleotide sequence of a target nucleic acid or fragment thereof may be an automated process. In some amplification reactions, capture probes may function as primers permitting the priming of a nucleotide synthesis reaction using a polynucleotide from the nucleic acid sample as a template. In this way, information regarding the sequence of the polynucleotides supplied to the array may be obtained. In some cases, polynucleotides hybridized to capture probes on the array may serve as sequencing templates if primers that hybridize to the polynucleotides bound to the capture probes and sequencing reagents are further supplied to the array.

Nucleic acid analysis methods may generate paired end reads on nucleic acid clusters. In some cases, a nucleic acid cluster may be immobilized on a sensor element, such as a surface. In some cases, paired end sequencing facilitates reading both the forward and reverse template strands of each cluster during one paired-end read. In some cases, template clusters may be amplified on the surface of a substrate (e.g. a flow-cell) by bridge amplification and sequenced by paired primers sequentially. Upon amplification of the template strands, a bridged double stranded structure may be produced. This may be treated to release a portion of one of the strands of each duplex from the surface. The single stranded nucleic acid may be available for sequencing, primer hybridization and cycles of primer extension. After the first sequencing run, the ends of the first single stranded template may be hybridized to the immobilized primers remaining from the initial cluster amplification procedure. The immobilized primers may be extended using the hybridized first single strand as a template to resynthesize the original double stranded structure. The double stranded structure may be treated to remove at least a portion of the first template strand to leave the resynthesized strand immobilized in single stranded form. The resynthesized strand may be sequenced to determine a second read, whose location originates from the opposite end of the original template fragment obtained from the fragmentation process.

Nucleic acid sequencing may be single-molecule sequencing or sequencing by synthesis. Sequencing may be massively parallel array sequencing (e.g., Illumina™ sequencing), which may be performed using template nucleic acid molecules immobilized on a support, such as a flow cell. A high-throughput sequencing method may sequence simultaneously (or substantially simultaneously) at least about 10,000, 100,000, 1 million, 10 million, 100 million, 1 billion, or more polynucleotide molecules. Sequencing methods may include, but are not limited to: pyrosequencing, sequencing-by synthesis, single-molecule sequencing, nanopore sequencing, semiconductor sequencing, sequencing-by-ligation, sequencing-by-hybridization, Digital Gene Expression (Helicos), massively parallel sequencing, e.g., Helicos, Clonal Single Molecule Array (Solexa/Illumina), sequencing using PacBio, SOLiD, Ion Torrent, or Nanopore platforms. Sequencing may comprise a first-generation sequencing method, such as Maxam-Gilbert or Sanger sequencing, or a high-throughput sequencing (e.g., next-generation sequencing or NGS) method.

The sequencing methods of the present disclosure may be able to detect germline susceptibility loci, somatic single nucleotide polymorphisms (SNPs), small insertion and deletion (indel) mutations, copy number variations (CNVs) and structural variants (SVs).

Furthermore, the sequencing methods of the present disclosure may quantify a nucleic acid, thus allowing sequence variations within an individual sample may be identified and quantified (e.g., a first percent of a gene is unmutated and a second percent of a gene present in a sample contains an indel).

Nucleic acid analysis methods may comprise physical analysis of nucleic acids collected from a biological sample. A method may distinguish nucleic acids based on their mass, post-transcriptional modification state (e.g., capping), histonylation, circularization (e.g., to detect extrachromosomal circular DNA elements), or melting temperature. For example, an assay may comprise restriction fragment length polymorphism (RFLP) or electrophoretic analysis on DNA collected from a biological sample. In some cases, post-transcriptional modification may comprise 5′ capping, 3′ cleavage, 3′ polyadenylation, splicing, or any combination thereof.

Nucleic acid analysis may also include sequence-specific interrogation. An assay for sequence-specific interrogation may target a particular sequence to determine its presence, absence or relative abundance in a biological sample. For example, an assay may comprise a southern blot, qPCR, fluorescence in situ hybridization (FISH), array-Comparative Genomic Hybridization (array-CGH), quantitative fluorescence PCR (QF-PCR), nanopore sequencing, sequencing by hybridization, sequencing by synthesis, sequencing by ligation, or capture by nucleic acid binding moieties (e.g., single stranded nucleotides or nucleic acid binding proteins) to determine the presence of a gene of interest (e.g., an oncogene) in a sample collected from a subject. An assay may also couple sequence specific collection with sequencing analysis. For example, an assay may comprise generating a particular sticky-end motif in nucleic acids comprising a specific target sequence, ligating an adaptor to nucleic acids with the particular sticky-end motif, and sequencing the adaptor-ligated nucleic acids to determine the presence or prevalence of mutations in a gene of interest.

### Genomic Information

The present disclosure provides various systems and methods for analyzing (e.g., detecting or sequencing) nucleic acids. In some cases, genotypic (or genomic) information may be obtained using some of the compositions and methods of the present disclosure. In some cases, genotypic information can refer to information about substances comprising a nucleotide and/or a nucleic acid component. In some cases, genotypic information may comprise epigenetic information. In some cases, epigenetic information may comprise histone modification, DNA methylation, accessibility of different regions in a genome, dynamics changes thereof, or any combination thereof. In some cases, genotypic information may comprise primary structure information, secondary structure information, tertiary structure information, or quaternary information about a nucleic acid. In some cases, genotypic information may comprise information about nucleic acid-ligand interactions, wherein a ligand may comprise any one of various biological molecules and substances that may be found in living organisms, such as, nucleotides, nucleic acids, amino acids, peptides, proteins, monosaccharides, polysaccharides, lipids, phospholipids, hormones, or any combination thereof. In some cases, genotypic information may comprise a rate or prevalence of apoptosis of a healthy cell or a diseased cell. In some cases, genotypic information may comprise a state of a cell, such as a healthy state or a diseased state. In some cases, genotypic information may comprise chemical modification information of a nucleic acid molecule. In some cases, a chemical modification may comprise methylation, demethylation, amination, deamination, acetylation, oxidation, oxygenation, reduction, or any combination thereof. In some cases, genotypic information may comprise information regarding from which type of cell a biological sample originates. In some cases, genotypic information may comprise information about an untranslated region of nucleic acids.

In some cases, genotypic information may comprise information about a single cell, a tissue, an organ, a system of tissues and/or organs (such as cardiovascular, respiratory, digestive, or nervous systems), or an entire multicellular organism. In some cases, genotypic information may comprise information about an individual (e.g., an individual human being or an individual bacterium), or a population of individuals (e.g., human beings with diagnosed with cancer or a colony of bacteria). Genotypic information may comprise information from various forms of life, including forms of life from the Archaea, the Bacteria, the Eukarya, the Protozoa, the Chromista, the Plantae, the Fungi, or from the Animalia. In some cases, genotypic information may comprise information from viruses. In some cases, genotypic information may comprise information relating exons and introns in the code of life. In some cases, genotypic information may comprise information regarding variations or mutations in the primary structure of nucleic acids, including base substitutions, deletions, insertions, or any combination thereof. In some cases, genotypic information may comprise information regarding the inclusion of non-canonical nucleobases in nucleic acids. In some cases, genotypic information may comprise information regarding variations or mutations in epigenetics.

In some cases, genotypic information may comprise information regarding variations in the primary structure, variations in the secondary structure, variations in the tertiary structure, or variations in the quaternary structure of peptides and/or proteins that one or more nucleic acids encode.

In some cases, a genomic variant may be detected using an assay. In some cases, a genomic variant can refer to a nucleic acid sequence originating from a DNA address(es) in a sample that comprises a sequence that is different a nucleic acid sequence originating from the same DNA address(es) in a reference sample. In some cases, a genomic variant may comprise a mutation such as an insertion mutation, deletion mutations, substitution mutation, copy number variations, transversions, translocations, inversion, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, abnormal changes in nucleic acid methylation infection, chromosal lesions, DNA lesions, or any combination thereof. In some cases, a set of genomic variants may comprise a single nucleotide polymorphism (SNP).

### Non-Specific Binding

A surface may bind biomolecules through variably selective adsorption (e.g., adsorption of biomolecules or biomolecule groups upon contacting the particle to a biological sample comprising the biomolecules or biomolecule groups, which adsorption is variably selective depending upon factors including e.g., physicochemical properties of the particle) or non-specific binding. Non-specific binding can refer to a class of binding interactions that exclude specific binding. Examples of specific binding may comprise protein-ligand binding interactions, antigen-antibody binding interactions, nucleic acid hybridizations, or a binding interaction between a template molecule and a target molecule wherein the template molecule provides a sequence or a 3D structure that favors the binding of a target molecule that comprise a complementary sequence or a complementary 3D structure, and disfavors the binding of a non-target molecule(s) that does not comprise the complementary sequence or the complementary 3D structure.

Non-specific binding may comprise one or a combination of a wide variety of chemical and physical interactions and effects. Non-specific binding may comprise electromagnetic forces, such as electrostatics interactions, London dispersion, Van der Waals interactions, or dipole-dipole interactions (e.g., between both permanent dipoles and induced dipoles). Non-specific binding may be mediated through covalent bonds, such as disulfide bridges. Non-specific binding may be mediated through hydrogen bonds. Non-specific binding may comprise solvophobic effects (e.g., hydrophobic effect), wherein one object is repelled by a solvent environment and is forced to the boundaries of the solvent, such as the surface of another object. Non-specific binding may comprise entropic effects, such as in depletion forces, or raising of the thermal energy above a critical solution temperature (e.g., a lower critical solution temperature). Non-specific binding may comprise kinetic effects, wherein one binding molecule may have faster binding kinetics than another binding molecule.

Non-specific binding may comprise a plurality of non-specific binding affinities for a plurality of targets (e.g., at least 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10,000, 20,000, 30,000, 40,000, 50,000 different targets adsorbed to a single particle). The plurality of targets may have similar non-specific binding affinities that are within about one, two, or three magnitudes (e.g., as measured by non-specific binding free energy, equilibrium constants, competitive adsorption, etc.). This may be contrasted with specific binding, which may comprise a higher binding affinity for a given target molecule than non-target molecules.

Biomolecules may adsorb onto a surface through non-specific binding on a surface at various densities. In some cases, biomolecules or proteins may adsorb at a density of at least about 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, or 1000 fg/mm². In some cases, biomolecules or proteins may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1000 pg/mm². In some cases, biomolecules or proteins may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1000 ng/mm². In some cases, biomolecules or proteins may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1000 μg/mm². In some cases, biomolecules or proteins may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1000 mg/mm². In some cases, biomolecules or proteins may adsorb at a density of at most about 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, or 1000 fg/mm². In some cases, biomolecules or proteins may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1000 μg/mm². In some cases, biomolecules or proteins may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1000 ng/mm². In some cases, biomolecules or proteins may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1000 μg/mm². In some cases, biomolecules or proteins may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1000 mg/mm².

Adsorbed biomolecules may comprise various types of proteins. In some cases, adsorbed proteins may comprise at least about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 types of proteins. In some cases, adsorbed proteins may comprise at most about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 types of proteins.

In some cases, proteins in a biological sample may comprise at least about 1, 2, 3, 4, 5, 6, 7, 8, 9, 10, 15, 20, 25, or 30 orders of magnitudes in concentration. In some cases, proteins in a biological sample may comprise at most about 1, 2, 3, 4, 5, 6, 7, 8, 9, 10, 15, 20, 25, or 30 orders of magnitudes in concentration.

### Composition Improving Assay

In some cases, a method of the present disclosure may comprise using a composition improving assay. In some cases, an untargeted assay may be a composition improving assay. In some cases, a composition improving assay may improve access to a subset of biomolecules in a biological sample. In some cases, a composition improving assay may improve detection to a subset of biomolecules in a biological sample. In some cases, a composition improving assay may improve identification to a subset of biomolecules in a biological sample. In some cases, the subset of biomolecules may be low-abundance biomolecules. In some cases, the subset of biomolecules may be rare biomolecules. In some cases, a dynamic range of a biological sample may be compressed using a composition improving assay. In some cases, a dynamic range may be compressed by at least about 1, 2, 3, 4, 5, 6, 7, 8, 9, 10, 11, 12, 13, 14, or 15 magnitudes.

In some cases, the composition improving assay may comprise providing one or more of surface regions comprising one or more surface types. In some cases, the composition improving assay may comprise contacting the biological sample with the one or more surface regions to yield a set of adsorbed biomolecules on the one or more surface regions. In some cases, the composition improving assay may comprise desorbing, from the one or more surface regions, at least a portion of the set of adsorbed biomolecules to yield the set of polyamino acids. In some cases, the composition improving assay may comprise contacting the biological sample with the one or more surface regions to capture a set of biomolecules on the one or more surface regions. In some cases, the composition improving assay may comprise releasing, from the one or more surface regions, at least a portion of the set of biomolecules to yield the set of polyamino acids. In some cases, the one or more surface regions are disposed on a single continuous surface. In some cases, the one or more surface regions are disposed on one or more discrete surfaces. In some cases, the one or more discrete surfaces are surfaces of one or more particles. In some cases, the one or more particles may comprise a nanoparticle. In some cases, the one or more particles may comprise a microparticle. In some cases, the one or more particles may comprise a porous particle. In some cases, the one or more particles may comprise a bifunctional, trifunctional, or N-functional particle.

In some cases, the composition improving assay may comprise providing a plurality of surface regions comprising a plurality of surface types. In some cases, the composition improving assay may comprise contacting the biological sample with the plurality of surface regions to yield a set of adsorbed biomolecules on the plurality of surface regions. In some cases, the composition improving assay may comprise desorbing, from the plurality of surface regions, at least a portion of the set of adsorbed biomolecules to yield the set of polyamino acids. In some cases, the composition improving assay may comprise contacting the biological sample with the plurality of surface regions to capture a set of biomolecules on the plurality of surface regions. In some cases, the composition improving assay may comprise releasing, from the plurality of surface regions, at least a portion of the set of biomolecules to yield the set of polyamino acids. In some cases, the plurality of surface regions are disposed on a single continuous surface. In some cases, the plurality of surface regions are disposed on a plurality of discrete surfaces. In some cases, the plurality of discrete surfaces are surfaces of a plurality of particles. In some cases, the plurality of particles may comprise a nanoparticle. In some cases, the plurality of particles may comprise a microparticle. In some cases, the plurality of particles may comprise a porous particle. In some cases, the plurality of particles may comprise a bifunctional, trifunctional, or N-functional particle.

### Machine Learning

A machine learning model can comprise one or more of various machine learning models. In some embodiments, the machine learning model can comprise one machine learning model. In some embodiments, the machine learning model can comprise a plurality of machine learning models. In some embodiments, the machine learning model can comprise a neural network model. In some embodiments, the machine learning model can comprise a random forest model. In some embodiments, the machine learning model can comprise a manifold learning model. In some embodiments, the machine learning model can comprise a hyperparameter learning model. In some embodiments, the machine learning model can comprise an active learning model.

A graph, graph model, and graphical model can refer to a method of conceptualizing or organizing information into a graphical representation comprising nodes and edges. In some embodiments, a graph can refer to the principle of conceptualizing or organizing data, wherein the data may be stored in a various and alternative forms such as linked lists, dictionaries, spreadsheets, arrays, in permanent storage, in transient storage, and so on, and is not limited to specific embodiments disclosed herein. In some embodiments, the machine learning model can comprise a graph model.

The machine learning model can comprise a variety of manifold learning algorithms. In some embodiments, the machine learning model can comprise a manifold learning algorithm. In some embodiments, the manifold learning algorithm is principal component analysis. In some embodiments, the manifold learning algorithm is a uniform manifold approximation algorithm. In some embodiments, the manifold learning algorithm is an isomap algorithm. In some embodiments, the manifold learning algorithm is a locally linear embedding algorithm. In some embodiments, the manifold learning algorithm is a modified locally linear embedding algorithm. In some embodiments, the manifold learning algorithm is a Hessian eigenmapping algorithm. In some embodiments, the manifold learning algorithm is a spectral embedding algorithm. In some embodiments, the manifold learning algorithm is a local tangent space alignment algorithm. In some embodiments, the manifold learning algorithm is a multi-dimensional scaling algorithm. In some embodiments, the manifold learning algorithm is a t-distributed stochastic neighbor embedding algorithm (t-SNE). In some embodiments, the manifold learning algorithm is a Barnes-Hut t-SNE algorithm.

The terms reducing, dimensionality reduction, projection, component analysis, feature space reduction, latent space engineering, feature space engineering, representation engineering, or latent space embedding can refer to a method of transforming a given input data with an initial number of dimensions to another form of data that has fewer dimensions than the initial number of dimensions. In some embodiments, the terms can refer to the principle of reducing a set of input dimensions to a smaller set of output dimensions.

The term normalizing can refer to a collection of methods for adjusting a dataset to align the dataset to a common scale. In some embodiments, a normalizing method can comprise multiplying a portion or the entirety of a dataset by a factor. In some embodiments, a normalizing method can comprise adding or subtracting a constant from a portion or the entirety of a dataset. In some embodiments, a normalizing method can comprise adjusting a portion or the entirety of a dataset to a known statistical distribution. In some embodiments, a normalizing method can comprise adjusting a portion or the entirety of a dataset to a normal distribution. In some embodiments, a normalizing method can comprise adjusting the dataset so that the signal strength of a portion or the entirety of a dataset is about the same.

Converting can comprise one or more steps of various of conversions of data. In some embodiments, converting can comprise normalizing data. In some embodiments, converting can comprise performing a mathematical operation that computes a score based on a distance between 2 points in the data. In some embodiments, the distance can comprise a distance between two edges in a graph. In some embodiments, the distance can comprise a distance between two nodes in a graph. In some embodiments, the distance can comprise a distance between a node and an edge in a graph. In some embodiments, the distance can comprise a Euclidean distance. In some embodiments, the distance can comprise a non-Euclidean distance. In some embodiments, the distance can be computed in a frequency space. In some embodiments, the distance can be computed in Fourier space. In some embodiments, the distance can be computed in Laplacian space. In some embodiments, the distance can be computed in spectral space. In some embodiments, the mathematical operation can be a monotonic function based on the distance. In some embodiments, the mathematical operation can be a non-monotonic function based on the distance. In some embodiments, the mathematical operation can be an exponential decay function. In some embodiments, the mathematical operation can be a learned function.

In some embodiments, converting can comprise transforming a data in one representation to another representation. In some embodiments, converting can comprise transforming data into another form of data with less dimensions. In some embodiments, converting can comprise linearizing one or more curved paths in the data. In some embodiments, converting can be performed on data comprising data in Euclidean space. In some embodiments, converting can be performed on data comprising data in graph space. In some embodiments, converting can be performed on data in a discrete space. In some embodiments, converting can be performed on data comprising data in frequency space. In some embodiments, converting can transform data in discrete space to continuous space, continuous space to discrete space, graph space to continuous space, continuous space to graph space, graph space to discrete space, discrete space to graph space, or any combination thereof. In some embodiments, converting can comprise transforming data in discrete space into a frequency domain. In some embodiments, converting can comprise transforming data in continuous space into a frequency domain. In some embodiments, converting can comprise transforming data in graph space into a frequency domain.

In some embodiments, the methods of the disclosure further comprise reducing polyamino acid descriptors to a reduced descriptor space using a machine learning model. In some embodiments, the method further comprises clustering the reduced descriptor space to determine one or more groups of polyamino acid descriptors with similar features.

In some embodiments, reducing can comprise transforming a given input data with any initial number of dimensions to another form of data that has any number of dimensions fewer than the initial number of dimensions. In some embodiments, reducing can comprise transforming input data into another form of data with fewer dimensions. In some embodiments, reducing can comprise linearizing one or more curved paths in the input data to the output data. In some embodiments, reducing can be performed on data comprising data in Euclidean space. In some embodiments, reducing can be performed on data comprising data in graph space. In some embodiments, reducing can be performed on data in a discrete space. In some embodiments, reducing can transform data in discrete space to continuous space, continuous space to discrete space, graph space to continuous space, continuous space to graph space, graph space to discrete space, discrete space to graph space, or any combination thereof.

The terms clustering, cluster analysis, or generating modules can refer to a method of grouping samples in a dataset by some measure of similarity. Samples can be grouped in a set space, for example, element ‘a’ is in set ‘A’. Samples can be grouped in a continuous space, for example, element ‘a’ is a point in Euclidean space with distance ‘l’ away from the centroid of elements comprising cluster ‘A’. Samples can be grouped in a graph space, for example, element ‘a’ is highly connected to elements comprising cluster ‘A’. These terms can refer to the principle of organizing a plurality of elements into groups in some mathematical space based on some measure of similarity.

Clustering can comprise grouping any number of samples in a dataset by any quantitative measure of similarity. In some embodiments, clustering can comprise K-means clustering. In some embodiments, clustering can comprise hierarchical clustering. In some embodiments, clustering can comprise using random forest models. In some embodiments, clustering can comprise boosted tree models. In some embodiments, clustering can comprise using support vector machines. In some embodiments, clustering can comprise calculating one or more N−1 dimensional surfaces in N-dimensional space that partitions a dataset into clusters. In some embodiments, clustering can comprise distribution-based clustering. In some embodiments, clustering can comprise fitting a plurality of prior distributions over the data distributed in N-dimensional space. In some embodiments, clustering can comprise using density-based clustering. In some embodiments, clustering can comprise using fuzzy clustering. In some embodiments, clustering can comprise computing probability values of a data point belonging to a cluster. In some embodiments, clustering can comprise using constraints. In some embodiments, clustering can comprise using supervised learning. In some embodiments, clustering can comprise using unsupervised learning.

In some embodiments, clustering can comprise grouping samples based on similarity. In some embodiments, clustering can comprise grouping samples based on quantitative similarity. In some embodiments, clustering can comprise grouping samples based on one or more features of each sample. In some embodiments, clustering can comprise grouping samples based on one or more labels of each sample. In some embodiments, clustering can comprise grouping samples based on Euclidean coordinates. In some embodiments, clustering can comprise grouping samples based the features of the nodes and edges of each sample.

In some embodiments, comparing can comprise comparing between a first group and different second group. In some embodiments, a first or a second group can each independently be a cluster. In some embodiments, a first or a second group can each independently be a group of clusters. In some embodiments, comparing can comprise comparing between one cluster with a group of clusters. In some embodiments, comparing can comprise comparing between a first group of clusters with second group of clusters different than the first group. In some embodiments, one group can be one sample. In some embodiments, one group can be a group of samples. In some embodiments, comparing can comprise comparing between one sample versus a group of samples. In some embodiments, comparing can comprise comparing between a group of samples versus a group of samples.

The terms “minimize”, “maximize”, “optimize”, “reduce”, “decrease”, “increase”, and the like, when used in the context of training a machine learning algorithm, can refer to the process of adjusting one or more parameters of a machine learning algorithm such that the value of a loss function is adjusted towards a defined objective (e.g., minimizing a difference between a machine learning output and examples). It can be said that the loss function is being minimized when the objective is defined to minimize a loss function.

### Neural Network

In some embodiments, systems and methods of the present disclosure may comprise or comprise using a neural network. The neural network may comprise various architectures, loss functions, optimization algorithms, assumptions, and various other neural network design choices. In some embodiments, the neural network comprises an encoder. In some embodiments, the neural network comprises a decoder. In some embodiments, the neural network comprises a bottleneck architecture comprising the encoder and the decoder. In some embodiments, the bottleneck architecture comprises an autoencoder. In some embodiments, the neural network comprises a language model. In some embodiments, the neural network comprises a transformer model.

Various types of layers may be used a neural network. In some embodiments, the neural network comprises a convolutional layer. In some embodiments, the neural network comprises a densely connected layer. In some embodiments, the neural network comprises a skip connection. In some embodiments, the neural network may comprise graph convolutional layers. In some embodiments, the neural network may comprise message passing layers. In some embodiments, the neural network may comprise attention layers. In some embodiments, the neural network may comprise recurrent layers. In some embodiments, the neural network may comprise a gated recurrent unit. In some embodiments, the neural network may comprise reversible layers. In some embodiments, the neural network may comprise a neural network with a bottleneck layer. In some embodiments, the neural network may comprise residual blocks. In some embodiments, the neural network may comprise one or more dropout layers. In some embodiments, the neural network may comprise one or more locally connected layers. In some embodiments, the neural network may comprise one or more batch normalization layers. In some embodiments, the neural network may comprise one or more pooling layers. In some embodiments, the neural network may comprise one or more upsampling layers. In some embodiments, the neural network may comprise one or more max-pooling layers.

In some embodiments, the neural network comprises a graph model. In some embodiments, a graph, graph model, and graphical model can refer to a method that models data in a graphical representation comprising nodes and edges. In some embodiments, the data may be stored in a various and alternative forms such as linked lists, dictionaries, spreadsheets, arrays, in permanent storage, in transient storage, and so on, and is not limited to specific embodiments disclosed herein.

In some embodiments, the neural network may comprise an autoencoder. In some embodiments, the neural network may comprise a variational autoencoder. In some embodiments, the neural network may comprise a generative adversarial network. In some embodiments, the neural network may comprise a flow model. In some embodiments, the neural network may comprise an autoregressive model.

The neural network may comprise various activation functions. In some embodiments, an activation function may be a non-linearity. In some embodiments, the neural network may comprise one or more activation functions. In some embodiments, the neural network may comprise a ReLU, softmax, tanh, sigmoid, softplus, softsign, selu, elu, exponential, LeakyReLU, or any combination thereof. Various activation functions may be used with a neural network, without departing from the inventive concepts disclosed herein.

### Training

Various loss functions can be used to train the neural network. In some embodiments, the neural network may comprise a regression loss function. In some embodiments, the neural network may comprise a logistic loss function. In some embodiments, the neural network may comprise a variational loss. In some embodiments, the neural network may comprise a prior. In some embodiments, the neural network may comprise a Gaussian prior. In some embodiments, the neural network may comprise a non-Gaussian prior. In some embodiments, the neural network may comprise a Laplacian prior. In some embodiments, the neural network may comprise a zero-inflated prior. In some case, the neural network may comprise a zero-inflated Poisson prior. In some embodiments, the neural network may comprise a zero-inflated negative binomial prior. In some embodiments, the neural network may comprise a Gaussian posterior. In some embodiments, the neural network may comprise a non-Gaussian posterior. In some embodiments, the neural network may comprise a Laplacian posterior. In some embodiments, the neural network may comprise an adversarial loss. In some embodiments, the neural network may comprise a reconstruction loss. In some embodiments, the loss functions may be formulated to optimize a regression loss, an evidence-based lower bound, a maximum likelihood, Kullback-Leibler divergence, applied with various distribution functions such as Gaussians, non-Gaussian, mixtures of Gaussians, mixtures of logistic functions, and so on.

Various optimizers can be used to train the neural network. In some embodiments, the neural network may be trained with the Adam optimizer. In some embodiments, the neural network may be trained with the stochastic gradient descent optimizer. In some embodiments, the neural network may be trained with an active learning algorithm. A neural network may be trained with various loss functions whose derivatives may be computed to update one or more parameters of the neural network. A neural network may be trained with hyperparameter searching algorithms. In some embodiments, the neural network hyperparameters are optimized with Gaussian Processes.

Various training protocols can be used while training the neural network. In some embodiments, the neural network may be trained with train/validation/test data splits. In some embodiments, the neural network may be trained with k-fold data splits, with any positive integer for k.

Training the neural network can involve providing inputs to the untrained neural network to generate predicted outputs, comparing the predicted outputs to the expected outputs, and updating the neural network's parameters to account for the difference between the predicted outputs and the expected outputs. Based on the calculated difference, a gradient with respect to each parameter may be calculated by backpropagation to update the parameters of the neural network so that the output value(s) that the neural network computes are consistent with the examples included in the training set. This process may be iterated for a certain number of iterations or until some stopping criterion is met.

The trained algorithm may be trained with a plurality of independent training samples. Each of the independent training samples may comprise a biomolecule descriptor. In the case of a variational autoencoder (VAE), the training samples may comprise individual observed biomolecule descriptors (e.g., polyamino acid descriptors, such as feature intensities) and corresponding reconstructed biomolecule descriptors. The trained algorithm may be trained, at least in part, to optimize the accuracy of the reconstruction when compared to the original input data.

After training the VAE, the encoder may be used to generate encodings (e.g., latent representations or latent descriptors) of biomolecule descriptors. Compared to the original or reconstructed descriptors, the latent descriptors may comprise certain properties. In some cases, the latent descriptors may comprise a reduced noise compared to the original descriptor. Without wishing to be bound by a particular theory, because the latent representation generally comprises fewer dimensions than the input feature, the autoencoder may “learn” during training to only capture in the latent representation those patterns in the input data which are significant (e.g., important for accurate reconstruction) while ignoring those that are less important. The latent space may additionally learn a continuous representation of the input data. For example, original biomolecule descriptors which are similar to one another may be close to one another in the latent space while those which are dissimilar to one another may be far apart in the latent space.

### Biomolecule Descriptors

Systems and methods as disclosed herein may ingest, operate on, transform, encode, decode, or output one or more biomolecule descriptors. Biomolecule descriptors may comprise any numerical or categorical data associated with a biomolecule. In some cases, a biomolecule descriptor comprises proteomic information as described herein. In some cases, a biomolecule descriptor comprises genomic information as described herein. In some cases, a biomolecule descriptor comprises transcriptomic information as described herein.

As used herein, “proteomic analysis”, “protein analysis”, and the like, may refer to any system or method for analyzing proteins in a sample, including the systems and methods disclosed herein. The present disclosure systems and methods for assaying using one or more surface. In some cases, a surface may comprise a surface of a high surface-area material, such as nanoparticles, particles, or porous materials. As used herein, a “surface” may refer to a surface for assaying polyamino acids. When a particle composition, physical property, or use thereof is described herein, it shall be understood that a surface of the particle may comprise the same composition, the same physical property, or the same use thereof, in some cases. Similarly, when a surface composition, physical property, or use thereof is described herein, it shall be understood that a particle may comprise the surface to comprise the same composition, the same physical property, or the same use thereof.

Materials for particles and surfaces may include metals, polymers, magnetic materials, and lipids. In some cases, magnetic particles may be iron oxide particles. Examples of metallic materials include any one of or any combination of gold, silver, copper, nickel, cobalt, palladium, platinum, iridium, osmium, rhodium, ruthenium, rhenium, vanadium, chromium, manganese, niobium, molybdenum, tungsten, tantalum, iron, cadmium, or any alloys thereof. In some cases, a particle disclosed herein may be a magnetic particle, such as a superparamagnetic iron oxide nanoparticle (SPION). In some cases, a magnetic particle may be a ferromagnetic particle, a ferrimagnetic particle, a paramagnetic particle, a superparamagnetic particle, or any combination thereof (e.g., a particle may comprise a ferromagnetic material and a ferrimagnetic material).

The present disclosure describes panels of particles or surfaces. In some cases, a panel may comprise more than one distinct surface types. Panels described herein can vary in the number of surface types and the diversity of surface types in a single panel. For example, surfaces in a panel may vary based on size, polydispersity, shape and morphology, surface charge, surface chemistry and functionalization, and base material. In some cases, panels may be incubated with a sample to be analyzed for polyamino acids, polyamino acid concentrations, nucleic acids, nucleic acid concentrations, or any combination thereof. In some cases, polyamino acids in the sample adsorb to distinct surfaces to form one or more adsorption layers of biomolecules. The identity of the biomolecules and concentrations thereof in the one or more adsorption layers may depend on the physical properties of the distinct surfaces and the physical properties of the biomolecules. Thus, each surface type in a panel may have differently adsorbed biomolecules due to adsorbing a different set of biomolecules, different concentrations of a particular biomolecules, or a combination thereof. Each surface type in a panel may have mutually exclusive adsorbed biomolecules or may have overlapping adsorbed biomolecules.

In some cases, panels disclosed herein can be used to identify the number of distinct biomolecules disclosed herein over a wide dynamic range in a given biological sample. For example, a panel may enrich a subset of biomolecules in a sample, which can be identified over a wide dynamic range at which the biomolecules are present in a sample (e.g., a plasma sample). In some cases, the enriching may be selective—e.g., biomolecules in the subset may be enriched but biomolecules outside of the subset may not enriched and/or be depleted. In some cases, the subset may comprise proteins having different post-translational modifications. For example, a first particle type in the particle panel may enrich a protein or protein group having a first post-translational modification, a second particle type in the particle panel may enrich the same protein or same protein group having a second post-translational modification, and a third particle type in the particle panel may enrich the same protein or same protein group lacking a post-translational modification. In some cases, the panel including any number of distinct particle types disclosed herein, enriches, and identifies a single protein or protein group by binding different domains, sequences, or epitopes of the protein or protein group. For example, a first particle type in the particle panel may enrich a protein or protein group by binding to a first domain of the protein or protein group, and a second particle type in the particle panel may enrich the same protein or same protein group by binding to a second domain of the protein or protein group. In some cases, a panel including any number of distinct particle types disclosed herein, may enrich and identify biomolecules over a dynamic range of at least 5, 6, 7, 8, 9, 10, 15, or 20 magnitudes. In some cases, a panel including any number of distinct particle types disclosed herein, may enrich and identify biomolecules over a dynamic range of at most 5, 6, 7, 8, 9, 10, 15, or 20 magnitudes.

A panel can have more than one surface type. Increasing the number of surface types in a panel can be a method for increasing the number of proteins that can be identified in a given sample.

A particle or surface may comprise a polymer. The polymer may constitute a core material (e.g., the core of a particle may comprise a particle), a layer (e.g., a particle may comprise a layer of a polymer disposed between its core and its shell), a shell material (e.g., the surface of the particle may be coated with a polymer), or any combination thereof. Examples of polymers include any one of or any combination of polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, or polyamines, a polyalkylene glycol (e.g., polyethylene glycol (PEG)), a polyester (e.g., poly(lactide-co-glycolide) (PLGA), polylactic acid, or polycaprolactone), or a copolymer of two or more polymers, such as a copolymer of a polyalkylene glycol (e.g., PEG) and a polyester (e.g., PLGA). The polymer may comprise a cross link. A plurality of polymers in a particle may be phase separated or may comprise a degree of phase separation.

Examples of lipids that can be used to form the particles or surfaces of the present disclosure include cationic, anionic, and neutrally charged lipids. For example, particles and/or surfaces can be made of any one of or any combination of dioleoylphosphatidylglycerol (DOPG), diacylphosphatidylcholine, diacylphosphatidylethanolamine, ceramide, sphingomyelin, cephalin, cholesterol, cerebrosides and diacylglycerols, dioleoylphosphatidylcholine (DOPC), dimyristoylphosphatidylcholine (DMPC), and dioleoylphosphatidylserine (DOPS), phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), lecithin, lysolecithin, phosphatidylethanolamine, lysophosphatidylethanolamine, dioleoylphosphatidylethanolamine (DOPE), dipalmitoyl phosphatidyl ethanolamine (DPPE), dimyristoylphosphoethanolamine (DMPE), distearoyl-phosphatidyl-ethanolamine (DSPE), palmitoyloleoyl-phosphatidylethanolamine (POPE) palmitoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleyolphosphatidylglycerol (POPG), 16-O-monomethyl PE, 16-O-dimethyl PE, 18-1-trans PE, palmitoyloleoyl-phosphatidylethanolamine (POPE), 1-stearoyl-2-oleoyl-phosphatidyethanolamine (SOPE), phosphatidylserine, phosphatidylinositol, sphingomyelin, cephalin, cardiolipin, phosphatidic acid, cerebrosides, dicetylphosphate, cholesterol, and any combination thereof.

A particle panel may comprise a combination of particles with silica and polymer surfaces. For example, a particle panel may comprise a SPION coated with a thin layer of silica, a SPION coated with poly(dimethyl aminopropyl methacrylamide) (PDMAPMA), and a SPION coated with poly(ethylene glycol) (PEG). A particle panel consistent with the present disclosure could also comprise two or more particles selected from the group consisting of silica coated SPION, an N-(3-Trimethoxysilylpropyl) diethylenetriamine coated SPION, a PDMAPMA coated SPION, a carboxyl-functionalized polyacrylic acid coated SPION, an amino surface functionalized SPION, a polystyrene carboxyl functionalized SPION, a silica particle, and a dextran coated SPION. A particle panel consistent with the present disclosure may also comprise two or more particles selected from the group consisting of a surfactant free carboxylate microparticle, a carboxyl functionalized polystyrene particle, a silica coated particle, a silica particle, a dextran coated particle, an oleic acid coated particle, a boronated nanopowder coated particle, a PDMAPMA coated particle, a Poly(glycidyl methacrylate-benzylamine) coated particle, and a Poly(N-[3-(Dimethylamino)propyl]methacrylamide-co-[2-(methacryloyloxy)ethyl]dimethyl-(3-sulfopropyl)ammonium hydroxide, P(DMAPMA-co-SBMA) coated particle. A particle panel consistent with the present disclosure may comprise silica-coated particles, N-(3-Trimethoxysilylpropyl)diethylenetriamine coated particles, poly(N-(3-(dimethylamino)propyl) methacrylamide) (PDMAPMA)-coated particles, phosphate-sugar functionalized polystyrene particles, amine functionalized polystyrene particles, polystyrene carboxyl functionalized particles, ubiquitin functionalized polystyrene particles, dextran coated particles, or any combination thereof.

A particle panel consistent with the present disclosure may comprise a silica functionalized particle, an amine functionalized particle, a silicon alkoxide functionalized particle, a carboxylate functionalized particle, and a benzyl or phenyl functionalized particle. A particle panel consistent with the present disclosure may comprise a silica functionalized particle, an amine functionalized particle, a silicon alkoxide functionalized particle, a polystyrene functionalized particle, and a saccharide functionalized particle. A particle panel consistent with the present disclosure may comprise a silica functionalized particle, an N-(3-Trimethoxysilylpropyl)diethylenetriamine functionalized particle, a PDMAPMA functionalized particle, a dextran functionalized particle, and a polystyrene carboxyl functionalized particle. A particle panel consistent with the present disclosure may comprise 5 particles including a silica functionalized particle, an amine functionalized particle, a silicon alkoxide functionalized particle.

Distinct surfaces or distinct particles of the present disclosure may differ by one or more physicochemical property. The one or more physicochemical property is selected from the group consisting of: composition, size, surface charge, hydrophobicity, hydrophilicity, roughness, density surface functionalization, surface topography, surface curvature, porosity, core material, shell material, shape, and any combination thereof. The surface functionalization may comprise a macromolecular functionalization, a small molecule functionalization, or any combination thereof. A small molecule functionalization may comprise an aminopropyl functionalization, amine functionalization, boronic acid functionalization, carboxylic acid functionalization, alkyl group functionalization, N-succinimidyl ester functionalization, monosaccharide functionalization, phosphate sugar functionalization, sulfurylated sugar functionalization, ethylene glycol functionalization, streptavidin functionalization, methyl ether functionalization, trimethoxysilylpropyl functionalization, silica functionalization, triethoxylpropylaminosilane functionalization, thiol functionalization, PCP functionalization, citrate functionalization, lipoic acid functionalization, ethyleneimine functionalization. A particle panel may comprise a plurality of particles with a plurality of small molecule functionalizations selected from the group consisting of silica functionalization, trimethoxysilylpropyl functionalization, dimethylamino propyl functionalization, phosphate sugar functionalization, amine functionalization, and carboxyl functionalization.

A small molecule functionalization may comprise a polar functional group. Non-limiting examples of polar functional groups comprise carboxyl group, a hydroxyl group, a thiol group, a cyano group, a nitro group, an ammonium group, an imidazolium group, a sulfonium group, a pyridinium group, a pyrrolidinium group, a phosphonium group or any combination thereof. In some embodiments, the functional group is an acidic functional group (e.g., sulfonic acid group, carboxyl group, and the like), a basic functional group (e.g., amino group, cyclic secondary amino group (such as pyrrolidyl group and piperidyl group), pyridyl group, imidazole group, guanidine group, etc.), a carbamoyl group, a hydroxyl group, an aldehyde group and the like.

A small molecule functionalization may comprise an ionic or ionizable functional group. Non-limiting examples of ionic or ionizable functional groups comprise an ammonium group, an imidazolium group, a sulfonium group, a pyridinium group, a pyrrolidinium group, a phosphonium group. A small molecule functionalization may comprise a polymerizable functional group. Non-limiting examples of the polymerizable functional group include a vinyl group and a (meth)acrylic group. In some embodiments, the functional group is pyrrolidyl acrylate, acrylic acid, methacrylic acid, acrylamide, 2-(dimethylamino)ethyl methacrylate, hydroxyethyl methacrylate and the like.

A surface functionalization may comprise a charge. For example, a particle can be functionalized to carry a net neutral surface charge, a net positive surface charge, a net negative surface charge, or a zwitterionic surface. Surface charge can be a determinant of the types of biomolecules collected on a particle. Accordingly, optimizing a particle panel may comprise selecting particles with different surface charges, which may not only increase the number of different proteins collected on a particle panel, but also increase the likelihood of identifying a biological state of a sample. A particle panel may comprise a positively charged particle and a negatively charged particle. A particle panel may comprise a positively charged particle and a neutral particle. A particle panel may comprise a positively charged particle and a zwitterionic particle. A particle panel may comprise a neutral particle and a negatively charged particle. A particle panel may comprise a neutral particle and a zwitterionic particle. A particle panel may comprise a negative particle and a zwitterionic particle. A particle panel may comprise a positively charged particle, a negatively charged particle, and a neutral particle. A particle panel may comprise a positively charged particle, a negatively charged particle, and a zwitterionic particle. A particle panel may comprise a positively charged particle, a neutral particle, and a zwitterionic particle. A particle panel may comprise a negatively charged particle, a neutral particle, and a zwitterionic particle.

A particle may comprise a single surface such as a specific small molecule, or a plurality of surface functionalizations, such as a plurality of different small molecules. Surface functionalization can influence the composition of a particle's biomolecule corona. Such surface functionalization can include small molecule functionalization or macromolecular functionalization. A surface functionalization may be coupled to a particle material such as a polymer, metal, metal oxide, inorganic oxide (e.g., silicon dioxide), or another surface functionalization.

A surface functionalization may comprise a small molecule functionalization, a macromolecular functionalization, or a combination of two or more such functionalizations. In some cases, a macromolecular functionalization may comprise a biomacromolecule, such as a protein or a polynucleotide (e.g., a 100-mer DNA molecule). A macromolecular functionalization may comprise a protein, polynucleotide, or polysaccharide, or may be comparable in size to any of the aforementioned classes of species. In some cases, a surface functionalization may comprise an ionizable moiety. In some cases, a surface functionalization may comprise pKa of at least about 1, 2, 3, 4, 5, 6, 7, 8, 9, 10, 11, 12, 13, or 14. In some cases, a surface functionalization may comprise pKa of at most about 1, 2, 3, 4, 5, 6, 7, 8, 9, 10, 11, 12, 13, or 14. In some cases, a small molecule functionalization may comprise a small organic molecule such as an alcohol (e.g., octanol), an amine, an alkane, an alkene, an alkyne, a heterocycle (e.g., a piperidinyl group), a heteroaromatic group, a thiol, a carboxylate, a carbonyl, an amide, an ester, a thioester, a carbonate, a thiocarbonate, a carbamate, a thiocarbamate, a urea, a thiourea, a halogen, a sulfate, a phosphate, a monosaccharide, a disaccharide, a lipid, or any combination thereof. For example, a small molecule functionalization may comprise a phosphate sugar, a sugar acid, or a sulfurylated sugar.

In some cases, a macromolecular functionalization may comprise a specific form of attachment to a particle. In some cases, a macromolecule may be tethered to a particle via a linker. In some cases, the linker may hold the macromolecule close to the particle, thereby restricting its motion and reorientation relative to the particle or may extend the macromolecule away from the particle. In some cases, the linker may be rigid (e.g., a polyolefin linker) or flexible (e.g., a nucleic acid linker). In some cases, a linker may be at least about 0.5, 1, 2, 3, 4, 5, 6, 7, 8, 9, 10, 15, 20, 25, or 30 nm in length. In some cases, a linker may be at most about 0.5, 1, 2, 3, 4, 5, 6, 7, 8, 9, 10, 15, 20, 25, or 30 nm in length. As such, a surface functionalization on a particle may project beyond a primary corona associated with the particle. In some cases, a surface functionalization may also be situated beneath or within a biomolecule corona that forms on the particle surface. In some cases, a macromolecule may be tethered at a specific location, such as at a protein's C-terminus, or may be tethered at a number of possible sites. For example, a peptide may be covalent attached to a particle via any of its surface exposed lysine residues.

In some cases, a particle may be contacted with a biological sample (e.g., a biofluid) to form a biomolecule corona. In some cases, a biomolecule corona may comprise at least two biomolecules that do not share a common binding motif. The particle and biomolecule corona may be separated from the biological sample, for example by centrifugation, magnetic separation, filtration, or gravitational separation. The particle types and biomolecule corona may be separated from the biological sample using a number of separation techniques. Non-limiting examples of separation techniques include comprises magnetic separation, column-based separation, filtration, spin column-based separation, centrifugation, ultracentrifugation, density or gradient-based centrifugation, gravitational separation, or any combination thereof. A protein corona analysis may be performed on the separated particle and biomolecule corona. A protein corona analysis may comprise identifying one or more proteins in the biomolecule corona, for example by mass spectrometry. In some cases, a single particle type may be contacted with a biological sample. In some cases, a plurality of particle types may be contacted to a biological sample. In some cases, the plurality of particle types may be combined and contacted to the biological sample in a single sample volume. In some cases, the plurality of particle types may be sequentially contacted to a biological sample and separated from the biological sample prior to contacting a subsequent particle type to the biological sample. In some cases, adsorbed biomolecules on the particle may have compressed (e.g., smaller) dynamic range compared to a given original biological sample.

In some cases, the particles of the present disclosure may be used to serially interrogate a sample by incubating a first particle type with the sample to form a biomolecule corona on the first particle type, separating the first particle type, incubating a second particle type with the sample to form a biomolecule corona on the second particle type, separating the second particle type, and repeating the interrogating (by incubation with the sample) and the separating for any number of particle types. In some cases, the biomolecule corona on each particle type used for serial interrogation of a sample may be analyzed by protein corona analysis. The biomolecule content of the supernatant may be analyzed following serial interrogation with one or more particle types.

In some cases, a method of the present disclosure may identify a large number of unique biomolecules (e.g., proteins) in a biological sample (e.g., a biofluid). In some cases, a surface disclosed herein may be incubated with a biological sample to adsorb at least 1000, 2000, 3000, 4000, 5000, 6000, 7000, 8000, 9000, or 10000 unique biomolecules. In some cases, a surface disclosed herein may be incubated with a biological sample to adsorb at most 1000, 2000, 3000, 4000, 5000, 6000, 7000, 8000, 9000, or 10000 unique biomolecules. In some cases, a surface disclosed herein may be incubated with a biological sample to adsorb at least 1000, 2000, 3000, 4000, 5000, 6000, 7000, 8000, 9000, or 10000 unique biomolecule groups. In some cases, a surface disclosed herein may be incubated with a biological sample to adsorb at most 1000, 2000, 3000, 4000, 5000, 6000, 7000, 8000, 9000, or 10000 unique biomolecule groups. In some cases, several different types of surfaces can be used, separately or in combination, to identify large numbers of proteins in a particular biological sample. In other words, surfaces can be multiplexed in order to bind and identify large numbers of biomolecules in a biological sample.

In some cases, a method of the present disclosure may identify a large number of unique proteoforms in a biological sample. In some cases, a method may identify at least about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 unique proteoforms. In some cases, a method may identify at most about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 unique proteoforms. In some cases, a surface disclosed herein may be incubated with a biological sample to adsorb at least 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 unique proteoforms. In some cases, a surface disclosed herein may be incubated with a biological sample to adsorb at most 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 unique proteoforms. In some cases, several different types of surfaces can be used, separately or in combination, to identify large numbers of proteins in a particular biological sample. In other words, surfaces can be multiplexed in order to bind and identify large numbers of biomolecules in a biological sample.

Biomolecules collected on particles may be subjected to further analysis. In some cases, a method may comprise collecting a biomolecule adsorption layer (e.g., corona) or a subset of biomolecules from a biomolecule adsorption layer. In some cases, the collected biomolecule adsorption layer or the collected subset of biomolecules from the biomolecule adsorption layer may be subjected to further particle-based analysis (e.g., particle adsorption). In some cases, the collected biomolecule adsorption layer or the collected subset of biomolecules from the biomolecule adsorption layer may be purified or fractionated (e.g., by a chromatographic method). In some cases, the collected biomolecule adsorption layer or the collected subset of biomolecules from the biomolecule adsorption layer may be analyzed (e.g., by mass spectrometry). Analysis of the biomolecule adsorption layer (e.g., by a chromatographic method and/or mass spectrometry) may generate biomolecule descriptors indicative of the composition of the biomolecule adsorption layer for use in the methods and systems (e.g., for generating embeddings or classifying samples) described herein.

In some cases, the panels disclosed herein can be used to identify a number of proteins, peptides, protein groups, or protein classes using a protein analysis workflow described herein (e.g., a protein corona analysis workflow). In some cases, the panels disclosed herein can be used to identify at least 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 unique proteins. In some cases, the panels disclosed herein can be used to identify at most 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 unique proteins. In some cases, the panels disclosed herein can be used to identify at least 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 protein groups. In some cases, the panels disclosed herein can be used to identify at most 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 protein groups. In some cases, the panels disclosed herein can be used to identify at least 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, or 1000000 peptides. In some cases, the panels disclosed herein can be used to identify at most 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, or 1000000 peptides. In some cases, a biomolecule descriptor comprises a peptide (e.g., polyamino acid). In some cases, a peptide may be a tryptic peptide. In some cases, a biomolecule descriptor comprises a tryptic peptide. In some cases, a peptide may be a semi-tryptic peptide. In some cases, a biomolecule descriptor comprises a semi-tryptic peptide. In some cases, protein analysis may comprise contacting a sample to distinct surface types (e.g., a particle panel), forming adsorbed biomolecule layers on the distinct surface types, and identifying the biomolecules in the adsorbed biomolecule layers (e.g., by mass spectrometry). Feature intensities, as disclosed herein, may refer to the intensity of a discrete spike (“feature”) seen on a plot of mass to charge ratio versus intensity from a mass spectrometry run of a sample. In some cases, these features can correspond to variably ionized fragments of peptides and/or proteins. In some cases, using the data analysis methods described herein, feature intensities can be sorted into protein groups. In some cases, protein groups may refer to two or more proteins that are identified by a shared peptide sequence. In some cases, a protein group can refer to one protein that is identified using a unique identifying sequence. For example, if in a sample, a peptide sequence is assayed that is shared between two proteins (Protein 1: XYZZX and Protein 2: XYZYZ), a protein group could be the “XYZ protein group” having two members (protein 1 and protein 2). In some cases, if the peptide sequence is unique to a single protein (Protein 1), a protein group could be the “ZZX” protein group having one member (Protein 1). In some cases, each protein group can be supported by more than one peptide sequence. In some cases, protein detected or identified according to the instant disclosure can refer to a distinct protein detected in the sample (e.g., distinct relative other proteins detected using mass spectrometry). In some cases, analysis of proteins present in distinct biomolecule adsorption layers corresponding to the distinct surface types in a panel yields a high number of feature intensities. In some cases, this number decreases as feature intensities are processed into distinct peptides, further decreases as distinct peptides are processed into distinct proteins, and further decreases as peptides are grouped into protein groups (two or more proteins that share a distinct peptide sequence). In some cases, a biomolecule descriptor comprises a feature intensity. In some cases, a biomolecule descriptor comprises a protein or protein group.

In some cases, the methods disclosed herein include isolating one or more particle types from a sample or from more than one sample (e.g., a biological sample or a serially interrogated sample). The particle types can be rapidly isolated or separated from the sample using a magnet. Moreover, multiple samples that are spatially isolated can be processed in parallel. In some cases, the methods disclosed herein provide for isolating or separating a particle type from unbound protein in a sample. In some cases, a particle type may be separated by a variety of means, including but not limited to magnetic separation, centrifugation, filtration, or gravitational separation. In some cases, particle panels may be incubated with a plurality of spatially isolated samples, wherein each spatially isolated sample is in a well in a well plate (e.g., a 96-well plate). In some cases, the particle in each of the wells of the well plate can be separated from unbound protein present in the spatially isolated samples by placing the entire plate on a magnet. In some cases, this simultaneously pulls down the superparamagnetic particles in the particle panel. In some cases, the supernatant in each sample can be removed to remove the unbound protein. In some cases, these steps (incubate, pull down) can be repeated to effectively wash the particles, thus removing residual background unbound protein that may be present in a sample.

In some cases, the systems and methods disclosed herein may also elucidate protein classes or interactions of the protein classes. In some cases, a protein class may comprise a set of proteins that share a common function (e.g., amine oxidases or proteins involved in angiogenesis); proteins that share common physiological, cellular, or subcellular localization (e.g., peroxisomal proteins or membrane proteins); proteins that share a common cofactor (e.g., heme or flavin proteins); proteins that correspond to a particular biological state (e.g., hypoxia related proteins); proteins containing a particular structural motif (e.g., a cupin fold); proteins that are functionally related (e.g., part of a same metabolic pathway); or proteins bearing a post-translational modification (e.g., ubiquitinated or citrullinated proteins). In some cases, a protein class may contain at least 2 proteins, 5 proteins, 10 proteins, 20 proteins, 40 proteins, 60 proteins, 80 proteins, 100 proteins, 150 proteins, 200 proteins, or more. In some cases, a biomolecule descriptor comprises a protein class.

In some cases, the proteomic data of the biological sample can be identified, measured, and quantified using a number of different analytical techniques. For example, proteomic data can be generated using SDS-PAGE or any gel-based separation technique. In some cases, peptides and proteins can also be identified, measured, and quantified using an immunoassay, such as ELISA. In some cases, proteomic data can be identified, measured, and quantified using mass spectrometry, high performance liquid chromatography, LC-MS/MS, Edman Degradation, immunoaffinity techniques, and other protein separation techniques. In some cases, a biomolecule descriptor comprises proteomic data.

In some cases, an assay may comprise protein collection of particles, protein digestion, and mass spectrometric analysis (e.g., MS, LC-MS, LC-MS/MS). In some cases, the digestion may comprise chemical digestion, such as by cyanogen bromide or 2-Nitro-5-thiocyanatobenzoic acid (NTCB). In some cases, the digestion may comprise enzymatic digestion, such as by trypsin or pepsin. In some cases, the digestion may comprise enzymatic digestion by a plurality of proteases. In some cases, the digestion may comprise a protease selected from among the group consisting of trypsin, chymotrypsin, Glu C, Lys C, elastase, subtilisin, proteinase K, thrombin, factor X, Arg C, papaine, Asp N, thermolysine, pepsin, aspartyl protease, cathepsin D, zinc mealloprotease, glycoprotein endopeptidase, proline, aminopeptidase, prenyl protease, caspase, kex2 endoprotease, or any combination thereof. In some cases, the digestion may cleave peptides at random positions. In some cases, the digestion may cleave peptides at a specific position (e.g., at methionines) or sequence (e.g., glutamate-histidine-glutamate). In some cases, the digestion may enable similar proteins to be distinguished. For example, an assay may resolve 8 distinct proteins as a single protein group with a first digestion method, and as 8 separate proteins with distinct signals with a second digestion method. In some cases, the digestion may generate an average peptide fragment length of 8 to 15 amino acids. In some cases, the digestion may generate an average peptide fragment length of 12 to 18 amino acids. In some cases, the digestion may generate an average peptide fragment length of 15 to 25 amino acids. In some cases, the digestion may generate an average peptide fragment length of 20 to 30 amino acids. In some cases, the digestion may generate an average peptide fragment length of 30 to 50 amino acids.

In some cases, an assay may rapidly generate and analyze proteomic data. In some cases, beginning with an input biological sample (e.g., a buccal or nasal smear, plasma, or tissue), a method of the present disclosure may generate and analyze proteomic data in less than about 1, 2, 3, 4, 5, 6, 7, 8, 12, 16, 20, 24, or 48 hours. In some cases, the analyzing may comprise identifying a protein group. In some cases, the analyzing may comprise identifying a protein class. In some cases, the analyzing may comprise quantifying an abundance of a biomolecule, a peptide, a protein, protein group, or a protein class. In some cases, the analyzing may comprise identifying a ratio of abundances of two biomolecules, peptides, proteins, protein groups, or protein classes. In some cases, the analyzing may comprise identifying a biological state.

An example of a particle type of the present disclosure may be a carboxylate (Citrate) superparamagnetic iron oxide nanoparticle (SPION), a phenol-formaldehyde coated SPION, a silica-coated SPION, a polystyrene coated SPION, a carboxylated poly(styrene-co-methacrylic acid) coated SPION, a N-(3-Trimethoxysilylpropyl)diethylenetriamine coated SPION, a poly(N-(3-(dimethylamino)propyl) methacrylamide) (PDMAPMA)-coated SPION, a 1,2,4,5-Benzenetetracarboxylic acid coated SPION, a poly(Vinylbenzyltrimethylammonium chloride) (PVBTMAC) coated SPION, a carboxylate, PAA coated SPION, a poly(oligo(ethylene glycol) methyl ether methacrylate) (POEGMA)-coated SPION, a carboxylate microparticle, a polystyrene carboxyl functionalized particle, a carboxylic acid coated particle, a silica particle, a carboxylic acid particle of about 150 nm in diameter, an amino surface microparticle of about 0.4-0.6 μm in diameter, a silica amino functionalized microparticle of about 0.1-0.39 μm in diameter, a Jeffamine surface particle of about 0.1-0.39 μm in diameter, a polystyrene microparticle of about 2.0-2.9 μm in diameter, a silica particle, a carboxylated particle with an original coating of about 50 nm in diameter, a particle coated with a dextran based coating of about 0.13 μm in diameter, or a silica silanol coated particle with low acidity. In some cases, a particle may lack functionalized specific binding moieties for specific binding on its surface. In some cases, a particle may lack functionalized proteins for specific binding on its surface. In some cases, a surface functionalized particle does not comprise an antibody or a T cell receptor, a chimeric antigen receptor, a receptor protein, or a variant or fragment thereof. In some cases, the ratio between surface area and mass can be a determinant of a particle's properties. The particles disclosed herein can have surface area to mass ratios of 3 to 30 cm²/mg, 5 to 50 cm²/mg, 10 to 60 cm²/mg, 15 to 70 cm²/mg, 20 to 80 cm²/mg, 30 to 100 cm²/mg, 35 to 120 cm²/mg, 40 to 130 cm²/mg, 45 to 150 cm²/mg, 50 to 160 cm²/mg, 60 to 180 cm²/mg, 70 to 200 cm²/mg, 80 to 220 cm²/mg, 90 to 240 cm²/mg, 100 to 270 cm²/mg, 120 to 300 cm²/mg, 200 to 500 cm²/mg, 10 to 300 cm²/mg, 1 to 3000 cm²/mg, 20 to 150 cm²/mg, 25 to 120 cm²/mg, or from 40 to 85 cm²/mg. Small particles (e.g., with diameters of 50 nm or less) can have significantly higher surface area to mass ratios, stemming in part from the higher order dependence on diameter by mass than by surface area. In some cases (e.g., for small particles), the particles can have surface area to mass ratios of 200 to 1000 cm²/mg, 500 to 2000 cm²/mg g, 1000 to 4000 cm²/mg, 2000 to 8000 cm²/mg, or 4000 to 10000 cm²/mg. In some cases (e.g., for large particles), the particles can have surface area to mass ratios of 1 to 3 cm²/mg, 0.5 to 2 cm²/mg, 0.25 to 1.5 cm²/mg, or 0.1 to 1 cm²/mg. A particle may comprise a wide array of physical properties. A physical property of a particle may include composition, size, surface charge, hydrophobicity, hydrophilicity, amphipathicity, surface functionality, surface topography, surface curvature, porosity, core material, shell material, shape, zeta potential, and any combination thereof. A particle may have a core-shell structure. In some cases, a core material may comprise metals, polymers, magnetic materials, paramagnetic materials, oxides, and/or lipids. In some cases, a shell material may comprise metals, polymers, magnetic materials, oxides, and/or lipids.

In some cases, proteomic information or data can refer to information about substances comprising a peptide and/or a protein component. In some cases, proteomic information may comprise primary structure information, secondary structure information, tertiary structure information, or quaternary information about the peptide or a protein. In some cases, proteomic information may comprise information about protein-ligand interactions, wherein a ligand may comprise any one of various biological molecules and substances that may be found in living organisms, such as, nucleotides, nucleic acids, amino acids, peptides, proteins, monosaccharides, polysaccharides, lipids, phospholipids, hormones, or any combination thereof. In some cases, a biomolecule descriptor comprises proteomic information

In some cases, proteomic information may comprise information about a single cell, a tissue, an organ, a system of tissues and/or organs (such as cardiovascular, respiratory, digestive, or nervous systems), or an entire multicellular organism. In some cases, proteomic information may comprise information about an individual (e.g., an individual human being or an individual bacterium), or a population of individuals (e.g., human beings with diagnosed with cancer or a colony of bacteria). Proteomic information may comprise information from various forms of life, including forms of life from the Archaea, the Bacteria, the Eukarya, the Protozoa, the Chromista, the Plantae, the Fungi, or from the Animalia. In some cases, proteomic information may comprise information from viruses.

In some cases, proteomic information may comprise information relating exons and introns in the code of life. In some cases, proteomic information may comprise information regarding variations in the primary structure, variations in the secondary structure, variations in the tertiary structure, or variations in the quaternary structure of peptides and/or proteins. In some cases, proteomic information may comprise information regarding variations in the expression of exons, including alternative splicing variations, structural variations, or both. In some cases, proteomic information may comprise conformation information, post-translational modification information, chemical modification information (e.g., phosphorylation), cofactor (e.g., salts or other regulatory chemicals) association information, or substrate association information of peptides and/or proteins.

In some cases, proteomic information may comprise information related to various proteoforms in a sample. In some cases, a proteomic information may comprise information related to peptide variants, protein variants, or both. In some cases, a proteomic information may comprise information related to splicing variants, allelic variants, post-translation modification variants, or any combination thereof. In some cases, a biomolecule descriptor comprises proteoform data.

In some cases, splicing variant (in some cases also referred to as “alternative splicing” variants, “differential splicing” variants, or “alternative RNA splicing” variants) may refer to a protein that is expressed by an alternative splicing process. In some cases, an alternative splicing process may express one or more splicing variants from a set of exons via different combinations of exons. In some cases, a combination may comprise a different sequence of exons compared to another combination. In some cases, a combination may comprise a different subset of exons compared to another combination. In some cases, a splicing variant may comprise a reordered amino acid sequence of another splicing variant.

In some cases, an allelic variant may refer to a protein that is expressed from a gene comprising a mutation compared to a reference gene. In some cases, the reference gene may be the gene of a cell, an individual, or a population of individuals. In some cases, the mutation may be a base substitution, a base deletion, or a base insertion of a genetic sequence of the gene compared to a genetic reference of the reference gene. In some cases, an allelic variant may comprise an amino acid substitution in an amino acid sequence of another allelic variant.

In some cases, a post-translation modification may refer to a protein that is modified after expression. A protein may be modified by various enzymes. In some cases, an enzyme that can modify a protein may be a kinase, a protease, a ligase, a phosphatase, a transferase, a phosphotransferase, or any other enzyme for performing the any one of modifications disclosed herein.

In some cases, peptide variants or protein variants may comprise a post-translation modification. In some cases, the post-translational modification comprises acylation, alkylation, prenylation, flavination, amination, deamination, carboxylation, decarboxylation, nitrosylation, halogenation, sulfurylation, glutathionylation, oxidation, oxygenation, reduction, ubiquitination, SUMOylation, neddylation, myristoylation, palmitoylation, isoprenylation, farnesylation, geranylgeranylation, glypiation, glycosylphosphatidylinositol anchor formation, lipoylation, heme functionalization, phosphorylation, phosphopantetheinylation, retinylidene Schiff base formation, diphthamide formation, ethanolamine phosphoglycerol functionalization, hypusine formation, beta-Lysine addition, acetylation, formylation, methylation, amidation, amide bond formation, butyrylation, gamma-carboxylation, glycosylation, polysialylation, malonylation, hydroxylation, iodination, nucleotide addition, phosphate ester formation, phosphoramidate formation, adenylation, uridylylation, propionylation, pyroglutamate formation, gluthathionylation, sulfenylation, sulfinylation, sulfonylation, succinylation, sulfation, glycation, carbonylation, isopeptide bond formation, biotinylation, carbamylation, oxidation, pegylation, citrullination, deamidation, eliminylation, disulfide bond formation, proteolytic cleavage, isoaspartate formation, racemization, protein splicing, chaperon-assisted folding, or any combination thereof.

In some cases, proteomic information may be encoded as digital information. In some cases, the proteomic information may comprise one or more elements that represents the proteomic information. In some cases, an element may represent a primary structure information, secondary structure information, tertiary structure information, or quaternary information about a peptide or a protein. In some cases, an element may represent protein-ligand interactions for a peptide or a protein. In some cases, an element may represent a source of a peptide or protein (e.g., a specific cell, tissue, organ, organism, individual, or population of individuals). In some cases, an element may represent a type of proteoform. In some cases, an element may be a number, a vector, an array, or any other datatypes provided herein. In some cases, a biomolecule descriptor comprises the element or a plurality of elements.

As used herein, “genomic analysis”, “nucleic acid analysis”, and the like, may refer to any system or method for analyzing proteins in a sample, including the systems and methods disclosed herein. The present disclosure describes various compositions and methods for analyzing (e.g., detecting or sequencing) nucleic acids. In some cases, genotypic (or genomic) information may be obtained using some of the compositions and methods of the present disclosure. In some cases, genotypic information can refer to information about substances comprising a nucleotide and/or a nucleic acid component. In some cases, genotypic information may comprise epigenetic information. In some cases, epigenetic information may comprise histone modification, DNA methylation, accessibility of different regions in a genome, dynamics changes thereof, or any combination thereof. In some cases, genotypic information may comprise primary structure information, secondary structure information, tertiary structure information, or quaternary information about a nucleic acid. In some cases, genotypic information may comprise information about nucleic acid-ligand interactions, wherein a ligand may comprise any one of various biological molecules and substances that may be found in living organisms, such as, nucleotides, nucleic acids, amino acids, peptides, proteins, monosaccharides, polysaccharides, lipids, phospholipids, hormones, or any combination thereof. In some cases, genotypic information may comprise a rate or prevalence of apoptosis of a healthy cell or a diseased cell. In some cases, genotypic information may comprise a state of a cell, such as a healthy state or a diseased state. In some cases, genotypic information may comprise chemical modification information of a nucleic acid molecule. In some cases, a chemical modification may comprise methylation, demethylation, amination, deamination, acetylation, oxidation, oxygenation, reduction, or any combination thereof. In some cases, genotypic information may comprise information regarding from which type of cell a biological sample originates. In some cases, genotypic information may comprise information about an untranslated region of nucleic acids.

In some cases, genotypic information may comprise information about a single cell, a tissue, an organ, a system of tissues and/or organs (such as cardiovascular, respiratory, digestive, or nervous systems), or an entire multicellular organism. In some cases, genotypic information may comprise information about an individual (e.g., an individual human being or an individual bacterium), or a population of individuals (e.g., human beings with diagnosed with cancer or a colony of bacteria). Genotypic information may comprise information from various forms of life, including forms of life from the Archaea, the Bacteria, the Eukarya, the Protozoa, the Chromista, the Plantae, the Fungi, or from the Animalia. In some cases, genotypic information may comprise information from viruses.

In some cases, genotypic information may comprise information relating to exons and introns in the code of life. In some cases, genotypic information may comprise information regarding variations or mutations in the primary structure of nucleic acids, including base substitutions, deletions, insertions, or any combination thereof. In some cases, genotypic information may comprise information regarding the inclusion of non-canonical nucleobases in nucleic acids. In some cases, genotypic information may comprise information regarding variations or mutations in epigenetics.

In some cases, genotypic information may comprise information regarding variations in the primary structure, variations in the secondary structure, variations in the tertiary structure, or variations in the quaternary structure of peptides and/or proteins that one or more nucleic acids encode.

In some cases, the set of nucleic acids comprise an exome of the biological sample. In some cases, the set of nucleic acids comprise a genome, an epigenome, a transcriptome, a metabolome, a secretome, or any combination thereof. In some cases, the set of nucleic acids comprises a portion of the exome of the biological sample. In some cases, the set of nucleic acids comprise a portion of the genome, an epigenome, a transcriptome, a metabolome, a secretome, or any combination thereof. In some cases, the genotypic information comprises an exome sequence of the biological sample. In some cases, the genotypic information comprises one or more sequences of the genome, an epigenome, a transcriptome, a metabolome, a secretome, or any combination thereof.

Various sequencing methods and various sequencing reagents may be used to obtain genotypic information. In some cases, the sequencing methods disclosed herein may comprise enriching one or more nucleic acid molecules from a sample. This may comprise enrichment in solution, enrichment on a sensor element (e.g., a particle), enrichment on a substrate (e.g., a surface of an Eppendorf tube), or selective removal of a nucleic acid (e.g., by sequence-specific affinity precipitation). Enrichment may comprise amplification, including differential amplification of two or more different target nucleic acids. Differential amplification may be based on sequence, CG-content, or post-transcriptional modifications, such as methylation state. In some cases, enrichment may comprise hybridization methods, such as pull-down methods. For example, a substrate partition may comprise immobilized nucleic acids capable of hybridizing to nucleic acids of a particular sequence, and thereby capable of isolating particular nucleic acids from a complex biological solution. In some cases, hybridization may target genes, exons, introns, regulatory regions, splice sites, reassembly genes, among other nucleic acid targets. In some cases, hybridization can utilize a pool of nucleic acid probes that are designed to target multiple distinct sequences, or to tile a single sequence.

Enrichment may comprise a hybridization reaction and may generate a subset of nucleic acid molecules from a biological sample. Hybridization may be performed in solution, on a substrate surface (e.g., a wall of a well in a microwell plate), on a sensor element, or any combination thereof. A hybridization method may be sensitive for single nucleotide polymorphisms. For example, a hybridization method may comprise molecular inversion probes.

Enrichment may also comprise amplification. Suitable amplification methods include polymerase chain reaction (PCR), solid-phase PCR, RT-PCR, qPCR, multiplex PCR, touchdown PCR, nanoPCR, nested PCR, hot start PCR, helicase-dependent amplification, loop mediated isothermal amplification (LAMP), self-sustained sequence replication, nucleic acid sequence-based amplification, strand displacement amplification, rolling circle amplification, ligase chain reaction, and any other suitable amplification technique.

The sequencing may target a specific sequence or region of a genome. The sequencing may target a type of sequence, such as exons. In some cases, the sequencing comprises exome sequencing. In some cases, the sequencing comprises whole exome sequencing. The sequencing may target chromatinated or non-chromatinated nucleic acids. The sequencing may be sequence-nonspecific (e.g., provide a reading regardless of the target sequence). The sequencing may target a polymerase accessible region of the genome. The sequencing may target nucleic acids localized in a part of a cell, such as the mitochondria or the cytoplasm. The sequencing may target nucleic acids localized in a cell, tissue, or an organ. The sequencing may target RNA, DNA, any other nucleic acid, or any combination thereof.

‘Nucleic acid’ may refer to a polymeric form of nucleotides of any length, in single-, double- or multi-stranded form. A nucleic acid may comprise any combination of ribonucleotides, deoxyribonucleotides, and natural and non-natural analogues thereof, including 5-bromouracil, peptide nucleic acids, locked nucleotides, glycol nucleotides, threose nucleotides, dideoxynucleotides, 3′-deoxyribonucleotides, dideoxyribonucleotides, 7-deaza-GTP, fluorophores-bound nucleotides, thiol containing nucleotides, biotin linked nucleotides, methyl-7-guanosine, methylated nucleotides, inosine, thiouridine, pseudourdine, dihydrouridine, queuosine, and wyosine. A nucleic acid may comprise a gene, a portion of a gene, an exon, an intron, messenger RNA (mRNA), transfer RNA (tRNA), ribosomal RNA (rRNA), short interfering RNA (siRNA), short-hairpin RNA (shRNA), micro-RNA (miRNA), a ribozyme, cDNA, a recombinant nucleic acid, a branched nucleic acid, a plasmid, cell-free DNA (cfDNA), cell-free RNA (cfRNA), genomic DNA, mitochondrial DNA (mtDNA), circulating tumor DNA (ctDNA), long non-coding RNA, telomerase RNA, Piwi-interacting RNA, small nuclear RNA (snRNA), small interfering RNA, YRNA, circular RNA, small nucleolar RNA, or pseudogene RNA. A nucleic acid may comprise a DNA or RNA molecule. A nucleic acid may also have a defined 3-dimensional structure. In some cases, a nucleic acid may comprise a non-canonical nucleobase or a nucleotide, such as hypoxanthine, xanthine, 7-methylguanine, 5,6-dihydrouracil, 5-methylcytosine, or any combination thereof. Nucleic acids may also comprise non-nucleic acid molecules.

A nucleic acid may be derived from various sources. In some cases, a nucleic acid may be derived from an exosome, an apoptotic body, a tumor cell, a healthy cell, a virtosome, an extracellular membrane vesicle, a neutrophil extracellular trap (NET), or any combination thereof.

A nucleic acid may comprise various lengths. In some cases, a nucleic acid may comprise at least about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 nucleotides. In some cases, a nucleic acid may comprise at most about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 nucleotides.

Various reagents may be used for sequencing. In some cases, a reagent may comprise primers, oligonucleotides, switch oligonucleotides, adapters, amplification adapters, polymerases, dNTPs, co-factors, buffers, enzymes, ionic co-factors, ligase, reverse transcriptase, restriction enzymes, endonucleases, transposase, protease, proteinase K, DNase, RNase, lysis agents, lysozymes, achromopeptidase, lysostaphin, labiase, kitalase, lyticase, inhibitors, inactivating agents, chelating agents, EDTA, crowding agents, reducing agents, DTT, surfactants, TritonX-IOO, Tween 20, sodium dodecyl sulfate, sarcosyl, or any combination thereof.

Various methods for sequencing nucleic acids may be used. In some cases, sequencing may comprise sequencing a whole genome or portions thereof. Sequencing may comprise sequencing a whole genome, a whole exome, portions thereof (e.g., a panel of genes, including potentially coding and non-coding regions thereof). Sequencing may comprise sequencing a transcriptome or portion thereof. Sequencing may comprise sequencing an exome or portion thereof. Sequencing coverage may be optimized based on analytical or experimental setup, or desired sequencing footprint. In some cases, a nucleic acid sequencing method may comprise high-throughput sequencing, next-generation sequencing, flow sequencing, massively-parallel sequencing, shotgun sequencing, single-molecule real-time sequencing, ion semiconductor sequencing, electrophoretic sequencing, pyrosequencing, sequencing by synthesis, combinatorial probe anchor synthesis sequencing, sequencing by ligation, nanopore sequencing, GenapSys sequencing, chain termination sequencing, polony sequencing, 454 pyrosequencing, reversible terminated chemistry sequencing, heliscope single molecule sequencing, tunneling currents DNA sequencing, sequencing by hybridization, clonal single molecule array sequencing, sequencing with MS, DNA-seq, RNA-seq, ATAC-seq, methyl-seq, ChIP-seq, or any combination thereof. The sequencing methods of the present disclosure may involve sequence analysis of RNA. RNA sequences or expression levels may be analyzed by using a reverse transcription reaction to generate complementary DNA (cDNA) molecules from RNA for sequencing or by using reverse transcription polymerase chain reaction for quantification of expression levels. The sequencing methods of the present disclosure may detect RNA structural variants and isoforms, such as splicing variants and structural variants. The sequencing methods of the present disclosure may quantify RNA sequences or structural variants. In some cases, a sequencing may method comprise spatial sequencing, single-cell sequencing or any combination thereof.

In some cases, nucleic acids may be processed by standard molecular biology techniques for downstream applications. In some cases, nucleic acids may be prepared from nucleic acids isolated from a sample of the present disclosure. In some cases, the nucleic acids may subsequently be attached to an adaptor polynucleotide sequence, which may comprise a double stranded nucleic acid. In some cases, the nucleic acids may be end repaired prior to attaching to the adaptor polynucleotide sequences. In some cases, adaptor polynucleotides may be attached to one or both ends of the nucleotide sequences. In some cases, the same or different adaptor may be bound to each end of the fragment, thereby producing an “adaptor-nucleic acid-adaptor” construct. In some cases, a plurality of the same or different adaptor may be bound to each end of the fragment. In some cases, different adaptors may be attached to each end of the nucleic acid when adaptors are attached to both ends of the nucleic acid.

In some cases, an oligonucleotide tag complementary to a sequencing primer may be incorporated with adaptors attached to a target nucleic acid. For analysis of multiple samples, different oligonucleotide tags complementary to separate sequencing primers may be incorporated with adaptors attached to a target nucleic acid.

In some cases, an oligonucleotide index tag may also be incorporated with adaptors attached to a target nucleic acid. In cases in which deletion products are generated from a plurality of polynucleotides prior to hybridizing the deletion products to a nucleic acid immobilized on a structure (e.g., a sensor element such as a particle), polynucleotides corresponding to different nucleic acids of interest may first be attached to different oligonucleotide tags such that subsequently generated deletion products corresponding to different nucleic acids of interest may be grouped or differentiated. Consequently, deletion products derived from the same nucleic acid of interest may have the same oligonucleotide index tag such that the index tag identifies sequencing reads derived from the same nucleic acid of interest. Likewise, deletion products derived from different nucleic acids of interest may have different oligonucleotide index tags to allow them to be grouped or differentiated such as on a sensor element. Oligonucleotide index tags may range in length from about 5, 10, 15, 20, 25, 30, 35, 40, 45, 50, 75, to 100 nucleotides or base pairs, or any length in between.

In some cases, the oligonucleotide index tags may be added separately or in conjunction with a primer, primer binding site or other component. Conversely, a pair-end read may be performed, wherein the read from the first end may comprise a portion of the sequence of interest and the read from the other (second) end may be utilized as a tag to identify the fragment from which the first read originated.

In some cases, a sequencing read may be initiated from the point of incorporation of the modified nucleotide into an extended capture probe. In some cases, a sequencing primer may be hybridized to extended capture probes or their complements, which may be optionally amplified prior to initiating a sequence read and extended in the presence of natural nucleotides. In some cases, extension of the sequencing primer may stall at the point of incorporation of the first modified nucleotide incorporated in the template, and a complementary modified nucleotide may be incorporated at the point of stall using a polymerase capable of incorporating a modified nucleotide (e.g. TiTaq polymerase). In some cases, a sequencing read may be initiated at the first base after the stall or point of modified nucleotide incorporation. In a sequencing-by-synthesis method, a sequencing read may be initiated at the first base after the stall or point of modified nucleotide incorporation.

The present disclosure describes methods and compositions related to nucleic acid (polynucleotide) sequencing. Some methods of the present disclosure may provide for identification and quantification of nucleic acids in a subject or a sample. In some cases, the nucleotide sequence of a portion of a target nucleic acid or fragment thereof may be determined using a variety of methods and devices. Examples of sequencing methods include electrophoretic, sequencing by synthesis, sequencing by ligation, sequencing by hybridization, single-molecule sequencing, and real time sequencing methods. In some cases, the process to determine the nucleotide sequence of a target nucleic acid or fragment thereof may be an automated process. In some amplification reactions, capture probes may function as primers permitting the priming of a nucleotide synthesis reaction using a polynucleotide from the nucleic acid sample as a template. In this way, information regarding the sequence of the polynucleotides supplied to the array may be obtained. In some cases, polynucleotides hybridized to capture probes on the array may serve as sequencing templates if primers that hybridize to the polynucleotides bound to the capture probes and sequencing reagents are further supplied to the array.

Nucleic acid analysis methods may generate paired end reads on nucleic acid clusters. In some cases, a nucleic acid cluster may be immobilized on a sensor element, such as a surface. In some cases, paired end sequencing facilitates reading both the forward and reverse template strands of each cluster during one paired-end read. In some cases, template clusters may be amplified on the surface of a substrate (e.g. a flow-cell) by bridge amplification and sequenced by paired primers sequentially. Upon amplification of the template strands, a bridged double stranded structure may be produced. This may be treated to release a portion of one of the strands of each duplex from the surface. The single stranded nucleic acid may be available for sequencing, primer hybridization and cycles of primer extension. After the first sequencing run, the ends of the first single stranded template may be hybridized to the immobilized primers remaining from the initial cluster amplification procedure. The immobilized primers may be extended using the hybridized first single strand as a template to resynthesize the original double stranded structure. The double stranded structure may be treated to remove at least a portion of the first template strand to leave the resynthesized strand immobilized in single stranded form. The resynthesized strand may be sequenced to determine a second read, whose location originates from the opposite end of the original template fragment obtained from the fragmentation process.

Nucleic acid sequencing may be single-molecule sequencing or sequencing by synthesis. Sequencing may be massively parallel array sequencing (e.g., Illumina™ sequencing), which may be performed using template nucleic acid molecules immobilized on a support, such as a flow cell. A high-throughput sequencing method may sequence simultaneously (or substantially simultaneously) at least about 10,000, 100,000, 1 million, 10 million, 100 million, 1 billion, or more polynucleotide molecules. Sequencing methods may include, but are not limited to: pyrosequencing, sequencing-by synthesis, single-molecule sequencing, nanopore sequencing, semiconductor sequencing, sequencing-by-ligation, sequencing-by-hybridization, Digital Gene Expression (Helicos), massively parallel sequencing, e.g., Helicos, Clonal Single Molecule Array (Solexa/Illumina), sequencing using PacBio, SOLiD, Ion Torrent, or Nanopore platforms. Sequencing may comprise a first-generation sequencing method, such as Maxam-Gilbert or Sanger sequencing, or a high-throughput sequencing (e.g., next-generation sequencing or NGS) method.

The sequencing methods of the present disclosure may be able to detect germline susceptibility loci, somatic single nucleotide polymorphisms (SNPs), small insertion and deletion (indel) mutations, copy number variations (CNVs) and structural variants (SVs).

Furthermore, the sequencing methods of the present disclosure may quantify a nucleic acid, thus allowing sequence variations within an individual sample may be identified and quantified (e.g., a first percent of a gene is unmutated and a second percent of a gene present in a sample contains an indel).

Nucleic acid analysis methods may comprise physical analysis of nucleic acids collected from a biological sample. A method may distinguish nucleic acids based on their mass, post-transcriptional modification state (e.g., capping), histonylation, circularization (e.g., to detect extrachromosomal circular DNA elements), or melting temperature. For example, an assay may comprise restriction fragment length polymorphism (RFLP) or electrophoretic analysis on DNA collected from a biological sample. In some cases, post-transcriptional modification may comprise 5′ capping, 3′ cleavage, 3′ polyadenylation, splicing, or any combination thereof.

Nucleic acid analysis may also include sequence-specific interrogation. An assay for sequence-specific interrogation may target a particular sequence to determine its presence, absence or relative abundance in a biological sample. For example, an assay may comprise a southern blot, qPCR, fluorescence in situ hybridization (FISH), array-Comparative Genomic Hybridization (array-CGH), quantitative fluorescence PCR (QF-PCR), nanopore sequencing, sequencing by hybridization, sequencing by synthesis, sequencing by ligation, or capture by nucleic acid binding moieties (e.g., single stranded nucleotides or nucleic acid binding proteins) to determine the presence of a gene of interest (e.g., an oncogene) in a sample collected from a subject. An assay may also couple sequence specific collection with sequencing analysis. For example, an assay may comprise generating a particular sticky-end motif in nucleic acids comprising a specific target sequence, ligating an adaptor to nucleic acids with the particular sticky-end motif, and sequencing the adaptor-ligated nucleic acids to determine the presence or prevalence of mutations in a gene of interest.

The present disclosure provides various systems and methods for analyzing (e.g., detecting or sequencing) nucleic acids. In some cases, genotypic (or genomic) information may be obtained using some of the compositions and methods of the present disclosure. In some cases, genotypic information can refer to information about substances comprising a nucleotide and/or a nucleic acid component. In some cases, genotypic information may comprise epigenetic information. In some cases, epigenetic information may comprise histone modification, DNA methylation, accessibility of different regions in a genome, dynamics changes thereof, or any combination thereof. In some cases, genotypic information may comprise primary structure information, secondary structure information, tertiary structure information, or quaternary information about a nucleic acid. In some cases, genotypic information may comprise information about nucleic acid-ligand interactions, wherein a ligand may comprise any one of various biological molecules and substances that may be found in living organisms, such as, nucleotides, nucleic acids, amino acids, peptides, proteins, monosaccharides, polysaccharides, lipids, phospholipids, hormones, or any combination thereof. In some cases, genotypic information may comprise a rate or prevalence of apoptosis of a healthy cell or a diseased cell. In some cases, genotypic information may comprise a state of a cell, such as a healthy state or a diseased state. In some cases, genotypic information may comprise chemical modification information of a nucleic acid molecule. In some cases, a chemical modification may comprise methylation, demethylation, amination, deamination, acetylation, oxidation, oxygenation, reduction, or any combination thereof. In some cases, genotypic information may comprise information regarding from which type of cell a biological sample originates. In some cases, genotypic information may comprise information about an untranslated region of nucleic acids.

In some cases, genotypic information may comprise information about a single cell, a tissue, an organ, a system of tissues and/or organs (such as cardiovascular, respiratory, digestive, or nervous systems), or an entire multicellular organism. In some cases, genotypic information may comprise information about an individual (e.g., an individual human being or an individual bacterium), or a population of individuals (e.g., human beings with diagnosed with cancer or a colony of bacteria). Genotypic information may comprise information from various forms of life, including forms of life from the Archaea, the Bacteria, the Eukarya, the Protozoa, the Chromista, the Plantae, the Fungi, or from the Animalia. In some cases, genotypic information may comprise information from viruses. In some cases, genotypic information may comprise information relating exons and introns in the code of life. In some cases, genotypic information may comprise information regarding variations or mutations in the primary structure of nucleic acids, including base substitutions, deletions, insertions, or any combination thereof. In some cases, genotypic information may comprise information regarding the inclusion of non-canonical nucleobases in nucleic acids. In some cases, genotypic information may comprise information regarding variations or mutations in epigenetics.

In some cases, genotypic information may comprise information regarding variations in the primary structure, variations in the secondary structure, variations in the tertiary structure, or variations in the quaternary structure of peptides and/or proteins that one or more nucleic acids encode.

In some cases, a genomic variant may be detected using an assay. In some cases, a genomic variant can refer to a nucleic acid sequence originating from a DNA address(es) in a sample that comprises a sequence that is different a nucleic acid sequence originating from the same DNA address(es) in a reference sample. In some cases, a genomic variant may comprise a mutation such as an insertion mutation, deletion mutations, substitution mutation, copy number variations, transversions, translocations, inversion, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, abnormal changes in nucleic acid methylation infection, chromosal lesions, DNA lesions, or any combination thereof. In some cases, a set of genomic variants may comprise a single nucleotide polymorphism (SNP).

### Classification Using Machine Learning

In some cases, identifications of biomolecules may be processed using a machine learning algorithm. In some cases, the identifications of biomolecules may comprise identifications of nucleic acids, variants thereof, proteins, variants thereof, and any combination thereof. In some cases, the machine learning algorithm may be an unsupervised or self-supervised learning algorithm. In some cases, the machine learning algorithm may be trained to learn a latent representation of the identifications of the biomolecules. In some cases, the machine learning algorithm may be supervised learning algorithm. In some cases, the machine learning algorithm may be trained to learn to associate a given set of identifications with a value associated with a predetermined task. For example, the predetermined task may comprise determining a disease state associated with the given set of identifications, where the value may indicate the probability of the disease state being present in a subject associated with the given set of identifications.

In some cases, the method of determining a set of biomolecules associated with the disease or disorder and/or disease state can include the analysis of the biomolecule corona of at least two samples. This determination, analysis or statistical classification can be performed by methods, including, but not limited to, for example, a wide variety of supervised and unsupervised data analysis, machine learning, deep learning, and clustering approaches including hierarchical cluster analysis (HCA), principal component analysis (PCA), Partial least squares Discriminant Analysis (PLS-DA), random forest, logistic regression, decision trees, support vector machine (SVM), k-nearest neighbors, naive Bayes, linear regression, polynomial regression, SVM for regression, K-means clustering, and hidden Markov models, among others. In other words, the biomolecules in the corona of each sample are compared/analyzed with each other to determine with statistical significance what patterns are common between the individual corona to determine a set of biomolecules that is associated with the disease or disorder or disease state.

In some cases, machine learning algorithms can be used to construct models that accurately assign class labels to examples based on the input features that describe the example. In some case it may be advantageous to employ machine learning and/or deep learning approaches for the methods described herein. For example, machine learning can be used to associate the biomolecule corona with various disease states (e.g. no disease, precursor to a disease, having early or late stage of the disease, etc.). For example, in some cases, one or more machine learning algorithms can be employed in connection with the methods disclosed hereinto analyze data detected and obtained by the biomolecule corona and sets of biomolecules derived therefrom. For example, machine learning can be coupled with genomic and proteomic information obtained using the methods described herein to determine not only if a subject has a pre-stage of cancer, cancer or does not have or develop cancer, and also to distinguish the type of cancer.

In some cases, machine learning algorithms may also be used to associate the results from protein corona analysis and results from nucleic acid sequencing analysis and further associate any trends or correlations between proteins and nucleic acids to a biological state (e.g., disease state, health state, subtypes of disease such as stages of disease are cancer subtypes).

In some cases, machine learning may be used to cluster proteins detected using a plurality of surfaces. In some cases, a panel of surfaces may be used to assay proteins from one or more biological samples. In some cases, a surface in the panel of surfaces may comprise diverse physicochemical properties. In some cases, proteins detected by the panel of surfaces may be clustered using a clustering algorithm. In some cases, proteins detected by the panel of surfaces may be clustered based at least partially on the intensities of detected protein signals, particle chemical properties, protein structural and/or functional groups, or any combination thereof.

A panel of surfaces may comprise any number of surfaces. In some cases, a panel of surfaces may comprise at least about 1, 2, 3, 4, 5, 6, 7, 8, 9, 10, 20, 30, 40, 50, 60, 70, 80, 90, 100, 200, 300, 400, 500, 600, 700, 800, 900, or 1000 surfaces. In some cases, a panel of surfaces may comprise at most about 1, 2, 3, 4, 5, 6, 7, 8, 9, 10, 20, 30, 40, 50, 60, 70, 80, 90, 100, 200, 300, 400, 500, 600, 700, 800, 900, or 1000 surfaces.

Inputs to a machine learning algorithm may comprise various kinds of inputs. In some cases, an input may comprise a value that represents a physicochemical property of a surface used to assay a biomolecule. A physicochemical property of a particle may comprise various properties disclosed herein, which includes: charge, hydrophobicity, hydrophilicity, amphipathicity, coordinating, reaction class, surface free energy, various functional groups/modifications (e.g., sugar, polymer, amine, amide, epoxy, crosslinker, hydroxyl, aromatic, or phosphate groups). In some cases, an input may comprise a value that represents a parameter of a given assay. A parameter may comprise incubation conditions including temperature, incubation time, pH, buffer type, and any variables in performing an assay disclosed herein.

In some cases, a clustering algorithm can refer to a method of grouping samples in a dataset by some measure of similarity. In some cases, samples can be grouped in a set space, for example, element ‘a’ is in set ‘A’. In some cases, samples can be grouped in a continuous space, for example, element ‘a’ is a point in Euclidean space with distance ‘1’ away from the centroid of elements comprising cluster ‘A’. In some cases, samples can be grouped in a graph space, for example, element ‘a’ is highly connected to elements comprising cluster ‘A’. In some cases, clustering can refer to the principle of organizing a plurality of elements into groups in some mathematical space based on some measure of similarity.

In some cases, clustering can comprise grouping any number of biomolecules in a dataset by any quantitative measure of similarity. In some cases, clustering can comprise K-means clustering. In some cases, clustering can comprise hierarchical clustering. In some cases, clustering can comprise using random forest models. In some cases, clustering can comprise boosted tree models. In some cases, clustering can comprise using support vector machines. In some cases, clustering can comprise calculating one or more N−1 dimensional surfaces in N-dimensional space that partitions a dataset into clusters. In some cases, clustering can comprise distribution-based clustering. In some cases, clustering can comprise fitting a plurality of prior distributions over the data distributed in N-dimensional space. In some cases, clustering can comprise using density-based clustering. In some cases, clustering can comprise using fuzzy clustering. In some cases, clustering can comprise computing probability values of a data point belonging to a cluster. In some cases, clustering can comprise using constraints. In some cases, clustering can comprise using supervised learning. In some embodiments, clustering can comprise using unsupervised learning.

In some cases, clustering can comprise grouping biomolecules based on similarity. In some cases, clustering can comprise grouping biomolecules based on quantitative similarity. In some cases, clustering can comprise grouping biomolecules based on one or more features of each protein. In some cases, clustering can comprise grouping biomolecules based on one or more labels of each protein. In some cases, clustering can comprise grouping biomolecules based on Euclidean coordinates in a numerical representation of biomolecules. In some cases, clustering can comprise grouping biomolecules based on protein structural groups or functional groups (e.g., protein structures, substructures, or functional groups from protein databases such as Protein Data Bank or CATH Protein Structure Classification database). In some cases, a protein structural group or functional group may comprise protein primary structure, secondary structure, tertiary structure, or quaternary structure. In some cases, a protein structural group or functional group may be based at least partially on alpha helices, beta sheets, relative distribution of amino acids with different properties (e.g., aliphatic, aromatic, hydrophilic, acidic, basic, etc.), a structural families (e.g., TIM barrel and beta barrel fold), protein domains (e.g., Death effector domain). In some cases, a protein structural group or functional group may be based at least partially on functional or spatial properties (e.g., functional groups—group of immune globulins, cytokines, cytoskeletal biomolecules, etc.).

### Automated Systems

Some of the methods and compositions in the present disclosure may be integrated with an automated system. An advantage of integrating compositions and methods into an automated system is that experiments can be streamlined, saving users time and improving efficiency in a research, clinical, or an applied setting. An automated system can offer repeatability of experiments, faster turnaround, and better communication between researchers and clinicians sharing useful protocols that may be followed using the automated system. An automated system can be engineered to run numerous experiments in parallel, can enable high-throughput approaches, and can be used to generate data for some of the machine learning methods described herein.

An automated system for assaying a biological sample may comprise: one or more surfaces disposed on or in a substrate for contacting one or more biological samples comprising a plurality of biomolecules; a sample storage unit comprising the one or more biological samples; a loading unit that is operably coupled to the substrate and the sample storage unit; and a computer readable medium comprising machine-executable code that, upon execution by a processor, implements a method comprising: (a) transferring the biological sample or a portion thereof from the sample storage unit to the substrate using the loading unit; (b) directing the biological sample into contact with the composition to adsorb at least a portion of the plurality of biomolecules in the biological sample onto the surface.

In some cases, the substrate is a single well, a multi-well plate, a tube, a multi-tube apparatus, or a microfluidic device. In some cases, the automated system may comprise a plurality of substrates.

The substrate may comprise one or more of any of the various compositions described in the present disclosure. In some cases, the substrate comprises a plurality of compositions, wherein at least one subset of surfaces are comprised in one or more compositions. In some cases, at least one subset of surfaces may differ from another subset in at least one physicochemical property.

An automated system can run experiments with different biological samples at once. In some cases, the sample storage unit can comprise a plurality of different biological samples. In some cases, transferring of a biological sample can comprise transferring each of the plurality of different biological samples to a different well of a multi-well plate.

An automated system can run experiments with different portions of biological samples. In some cases, a biological sample comprises a plurality of portions. For instance, a portion may be a fraction of a fractionated biological sample. In some cases, a portion may be a subsection of a tissue sample or a fraction of a whole blood sample (e.g., a portion of a buffy coat). In some cases, a portion may be a supernatant of a biological sample lysate. A portion of a biological sample can be transferred into a well. A portion of a biological sample may be diluted (e.g., with an aqueous buffer such as pH 6 phosphate buffer). The biological sample may be diluted by at least 2-fold, at least 3-fold, at least 4-fold, at least 5-fold, at least 6-fold, at least 8-fold, at least 10-fold, at least 15-fold, or at least 20-fold. In some cases, the transfer may be performed simultaneously by the automated system.

An automated system can be configured to contact a biological sample with a particle composition for various amounts of time. In some cases, a biological sample can remain in contact with a composition for a time period of at least about 10 seconds. In some cases, a biological sample can remain in contact with a composition for a time period of at least about 10 seconds. In some cases, the time period is at least about 1 minute. In some cases, the time period is at least about 5 minutes.

An automated system can be configured to add steps or remove various experimental steps. An automated system can be configured to rearrange various experimental steps. In some cases, the automated system can be configured to run a wash step. For example, the automated system may be configured to wash a biomolecule corona with resuspension. In some cases, the automated system can be configured to run a step for washing biomolecule corona without resuspension. In some cases, the automated system can be configured to run a step for producing a lysate. For example, the automated system may sonicate or apply an electric field to lyse exosomes present in a biological sample. In some cases, the automated system can be configured to run a step for reducing a lysate. In some cases, the automated system can be configured to run a step for filtering a lysate. In some cases, the automated system can be configured to run a step for alkylating a lysate. In some cases, the automated system can be configured to run a step for denaturing a biomolecule corona. In some cases, the automated system can be configured to run a step for denaturing a biomolecule corona with a step-wise denaturing process. In some cases, the automated system can be configured to run a step to digest a biomolecule corona. The digestion step may comprise a protease such as trypsin, chymotrypsin, endoproteinase Asp-N, endoproteinase Arg-C, endoproteinase Lys-C, pepsin, thermolysin, elastase, papain, proteinase K, subtilisin, clostripain, carboxypeptidase, cathepsin C, or any combination thereof. The digestion step may comprise a chemical peptide cleavage agent, such as cyanogen bromide. The automated system may be configured to run a series of digestion steps, which may comprise different conditions, proteases, or chemical cleavage agents. A digestion step may use at most 50 ng/mL, at most 100 ng/mL, at most 200 ng/mL, at most 500 ng/mL, at most 1 μg/mL, at most 2 μg/mL, at most 5 μg/mL, at most 10 μg/mL, at most 25 μg/mL, at most 50 μg/mL, at most 100 μg/mL, at most 200 μg/mL, or at most 500 μg/mL of a protease. A digestion step may utilize at least 500 μg/mL, at least 200 μg/mL, at least 100 μg/mL, at least 50 μg/mL, at least 25 μg/mL, at least 10 μg/mL, at least 5 μg/mL, at least 2 μg/mL, at least 1 μg/mL, at least 500 ng/mL, at least 200 ng/mL, at least 100 ng/mL or at least 50 ng/mL of a protease. In some cases, the automated system can be configured to run a step to digest a biomolecule corona with trypsin at a concentration of at least about 200 nanograms per milliliter (ng/mL) to about 200 micrograms per milliliter (g/mL). In some cases, the automated system can be configured to run a step to digest a biomolecule corona with trypsin at a concentration of at least about 100 micrograms per milliliter (g/mL) to about 0.1 g/L. In some cases, the automated system can be configured to run a step to digest a biomolecule corona with lysC at a concentration of at least about 200 nanograms per milliliter (ng/mL) to about 200 micrograms per milliliter (g/mL). In some cases, the automated system can be configured to run a step to digest a biomolecule corona with lysC at a concentration of at least about 20 micrograms per milliliter (g/mL) to about 0.02 g/L. In some cases, the digestion step is performed for at most 3 hours. In some cases, the digestion step is performed for at most 1 hour. In some cases, the digestion step is performed for at most 30 minutes. In some cases, the digestion step generates peptides with an average mass of at least 1000 Da, at least 2000 Da, at least 3000 Da, at least 4000 Da, at least 5000 Da, at least 6000 Da, at least 8000 Da, or at least 10000 Da. In some cases, the digestion step generates peptides with an average mass of at most 10000 Da, at most 8000 Da, at most 6000 Da, at most 5000 Da, at most 4000 Da, at most 3000 Da, at most 2000 Da, or at most 1000 Da. In some cases, the digestion step generates peptides with an average mass of about 1000 Da to about 4000 Da. In some cases, the digestion step is preceded by elution of at least a subset of biomolecules or biomolecule groups from a biomolecule corona, for example such that the biomolecules or biomolecule groups are digested in solution. The elution may comprise dilution, heating, physical perturbation, addition of a chemical agent (e.g., a mild chaotropic agent), or any combination thereof.

In some cases, the automated system can be configured to elute a biomolecule corona or a portion of a biomolecule corona (e.g., selectively elute the soft portion of a biomolecule corona from a particle while leaving the hard portion of the biomolecule corona adsorbed to the particle). In some cases, the automated system can be configured to perform liquid chromatography on a biomolecule corona. In some cases, the automated system can be configured to separate a portion of a composition from a portion of the biological sample. In some cases, the automated system can be configured to separate a portion of a composition from a portion of the biological sample using a magnetic field. In some cases, the automated system can be configured run a proteomic experiment. In some cases, the automated system can be configured run a genomic experiment. In some cases, the automated system can be configured run a proteogenomic experiment. In some cases, the automated system can be configured run a mass spectroscopy experiment. In some cases, the automated system can be configured run a sequencing experiment.

An automated system can be configured run various experimental steps at various temperatures. In some cases, an automated system can be configured to run an experimental step at about −20, −19, −18, −17, −16, −15, −14, −13, −12, −11, −10, −9, −8, −7, −6, −5, −4, −3, −2, −1, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100° C.

An automated system can be configured run various experimental steps for various durations of time. In some cases, an automated system can be configured to run an experimental step at least about 1, 2, 3, 4, 5, 6, 7, 8, 9, 10, 15, 20, 30, 40, 50, or 60 minutes. In some cases, an automated system can be configured to run an experimental step at least about 1, 2, 3, 4, 5, 6, 7, 8, 9, or 10 hours. In some cases, an automated system can be configured to run an experimental step at most about 1, 2, 3, 4, 5, 6, 7, 8, 9, 10, 15, 20, 30, 40, 50, or 60 minutes. In some cases, an automated system can be configured to run an experimental step at most about 1, 2, 3, 4, 5, 6, 7, 8, 9, or 10 hours.

In some cases, the eluting step may comprise eluting with at most about 2× in volume of solution. In some cases, the eluting step may comprise eluting with at most about 4× in volume of solution. In some cases, the eluting step may comprise eluting with at most about 8× in volume of solution. In some cases, the eluting step may comprise eluting with at most about 16× in volume of solution. In some cases, the eluting comprises dilution. The dilution may be no more than 20-fold, no more than 10-fold, no more than 8-fold, no more than 5-fold, no more than 2-fold, or no more than 1.5-fold dilution. The elution may comprise a physical perturbation such as heating, sonication, shaking, or stirring. In some cases, the eluting comprises releasing an intact biomolecule (e.g., an intact protein) from the particle.

In some cases, the automated apparatus may perform solid phase extraction. The solid phase extraction may separate analytes (e.g., peptides digested from biomolecule corona proteins) from reagents (e.g., proteases), biomacromolecules and supramolecular biological structures (e.g., ribosomes and portions of cell walls), and species not amenable to downstream analysis (e.g., analytes incompatible with a liquid chromatography column). In some cases, the solid phase extraction utilizes a solid phase extraction plate comprising TF, iST, or C18. The solid phase extraction may be performed above atmospheric pressure. The pressure may be at least 25 pounds per square inch (psi), at least about 50 psi, at least about 100 psi, at least about 200 psi, at least about 300 psi, at least about 400 psi, or at least about 500 psi. In some cases, the solid phase extraction step may comprise eluting from a solid phase extraction plate with at most about 10, 20, 30, 40, 50, 60, 70, 80, 90, or 100 psi. In some cases, the solid phase extraction step may comprise eluting from a solid phase extraction plate with at least about 10, 20, 30, 40, 50, 60, 70, 80, 90, or 100 psi.

An automated system can comprise using a set of barcodes to identify biological samples, dry compositions, experimental steps, a substrate, a partition or volume within a substrate (e.g., a plasticware substrate), or reagents. An automated system may be configured to transfer a substrate based at least partially on a substrate (e.g., plateware) barcode. For example, the automated system may transfer a multi-well plate from a heater to a magnet array to immobilize magnetic particles contained in volumes of the multi-well plate. An automated system may be configured to transfer dry compositions based at least partially on a dry composition barcode. An automated system may be configured to transfer biological samples based at least partially on a biological sample barcode. An automated system may be configured to transfer samples and/or reagents between partitions or volumes of a substrate. An automated system may be configured to transfer reagents based at least partially on a reagent barcode. An automated system may be configured to set up experimental steps based at least partially on an experimental step barcode.

In some cases, a barcode may comprise information for plasticware, particle, reagent, kit, inventor management system, automated system, plate layout, or any combination thereof.

In some cases, an automated system may be in communication with a customer laboratory information management system (LIMS), an inventory management system, a MS machine, a personal computer, the cloud, the internet, or any combination thereof.

In some cases, an automated system may communicate barcodes, barcode information, plate layouts, experiment logs, MS files, biological sample information, analytical results of proteomic or genomic assays, or any combination thereof.

### Computer Systems

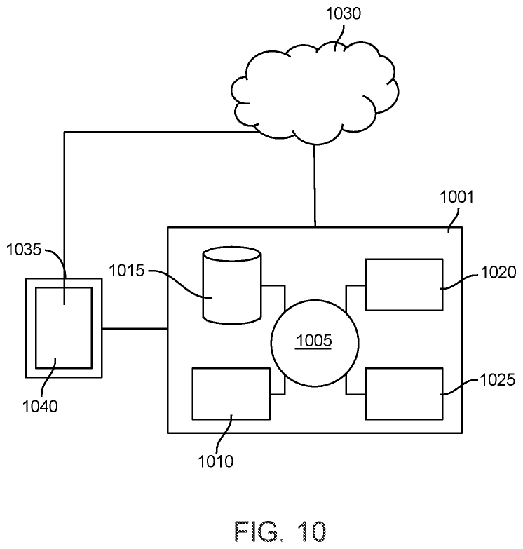

The present disclosure provides computer systems that are programmed to implement methods of the disclosure. FIG. 10 shows a computer system 1001 that is programmed or otherwise configured to, for example, analyze, convert, and/or display omics data.

The computer system 1001 may regulate various aspects of analysis, calculation, and generation of the present disclosure, such as, for example, converting, analyzing, and/or displaying omics data. The computer system 1001 may be an electronic device of a user or a computer system that is remotely located with respect to the electronic device. The electronic device may be a mobile electronic device.

The computer system 1001 includes a central processing unit (CPU, also “processor” and “computer processor” herein) 1005, which may be a single core or multi core processor, or a plurality of processors for parallel processing. The computer system 1001 also includes memory or memory location 1010 (e.g., random-access memory, read-only memory, flash memory), electronic storage unit 1015 (e.g., hard disk), communication interface 1020 (e.g., network adapter) for communicating with one or more other systems, and peripheral devices 1025, such as cache, other memory, data storage and/or electronic display adapters. The memory 1010, storage unit 1015, interface 1020 and peripheral devices 1025 are in communication with the CPU 1005 through a communication bus (solid lines), such as a motherboard. The storage unit 1015 may be a data storage unit (or data repository) for storing data. The computer system 1001 may be operatively coupled to a computer network (“network”) 1030 with the aid of the communication interface 1020. The network 1030 may be the Internet, an internet and/or extranet, or an intranet and/or extranet that is in communication with the Internet.

The network 1030 in some cases is a telecommunication and/or data network. The network 1030 may include one or more computer servers, which may enable distributed computing, such as cloud computing. For example, one or more computer servers may enable cloud computing over the network 1030 (“the cloud”) to perform various aspects of analysis, calculation, and generation of the present disclosure, such as, for example, converting, analyzing, and/or displaying omics data. Such cloud computing may be provided by cloud computing platforms such as, for example, Amazon Web Services (AWS), Microsoft Azure, Google Cloud Platform, and IBM cloud. The network 1030, in some cases with the aid of the computer system 1001, may implement a peer-to-peer network, which may enable devices coupled to the computer system 1001 to behave as a client or a server.

The CPU 1005 may comprise one or more computer processors and/or one or more graphics processing units (GPUs). The CPU 1005 may execute a sequence of machine-readable instructions, which may be embodied in a program or software. The instructions may be stored in a memory location, such as the memory 1010. The instructions may be directed to the CPU 1005, which may subsequently program or otherwise configure the CPU 1005 to implement methods of the present disclosure. Examples of operations performed by the CPU 1005 may include fetch, decode, execute, and writeback.

The CPU 1005 may be part of a circuit, such as an integrated circuit. One or more other components of the system 1001 may be included in the circuit. In some cases, the circuit is an application specific integrated circuit (ASIC).

The storage unit 1015 may store files, such as drivers, libraries and saved programs. The storage unit 1015 may store user data, e.g., user preferences and user programs. The computer system 1001 in some cases may include one or more additional data storage units that are external to the computer system 1001, such as located on a remote server that is in communication with the computer system 1001 through an intranet or the Internet.

The computer system 1001 may communicate with one or more remote computer systems through the network 1030. For instance, the computer system 1001 may communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user may access the computer system 1001 via the network 1030.

Methods as described herein may be implemented by way of machine (e.g., computer processor) executable code stored on an electronic storage location of the computer system 1001, such as, for example, on the memory 1010 or electronic storage unit 1015. The machine executable or machine readable code may be provided in the form of software. During use, the code may be executed by the processor 1005. In some cases, the code may be retrieved from the storage unit 1015 and stored on the memory 1010 for ready access by the processor 1005. In some situations, the electronic storage unit 1015 may be precluded, and machine-executable instructions are stored on memory 1010.

The code may be pre-compiled and configured for use with a machine having a processer adapted to execute the code, or may be compiled during runtime. The code may be supplied in a programming language that may be selected to enable the code to execute in a pre-compiled or as-compiled fashion.

Aspects of the systems and methods provided herein, such as the computer system 1001, may be embodied in programming. Various aspects of the technology may be thought of as “products” or “articles of manufacture” typically in the form of machine (or processor) executable code and/or associated data that is carried on or embodied in a type of machine readable medium. Machine-executable code may be stored on an electronic storage unit, such as memory (e.g., read-only memory, random-access memory, flash memory) or a hard disk. “Storage” type media may include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1001 may include or be in communication with an electronic display 1035 that comprises a user interface (UI) 1040 for providing, for example, converting, analyzing, and/or displaying omics data. Examples of UIs include, without limitation, a graphical user interface (GUI) and web-based user interface.

Methods and systems of the present disclosure may be implemented by way of one or more algorithms. An algorithm may be implemented by way of software upon execution by the central processing unit 1005. The algorithm can, for example, converting, analyzing, and/or displaying omics data.

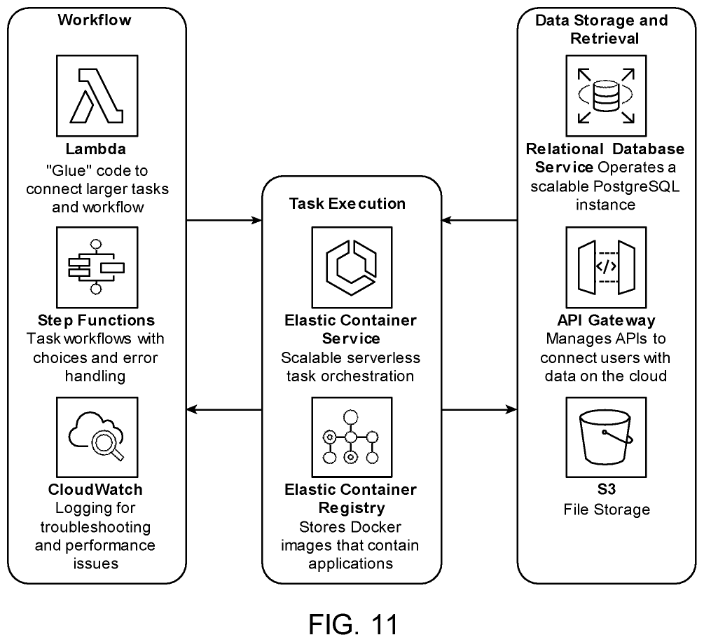

FIG. 11 schematically illustrates a cloud-based distributed computing environment, in accordance with some embodiments. In some embodiments, a computer system or a computer-implemented method of the present disclosure are configured to perform instructions on an event-driven and serverless platform. In some embodiments, instructions are performed with concurrency. In some embodiments, instructions are performed with scaling controls. In some embodiments, instructions can be packaged in container images. The container images can be configured to run on a variety of computing environments. In some embodiments, instructions comprise a signature for verifying integrity of the instructions. In some embodiments, instructions comprise a database proxy. The database proxy can manage a plurality of database connections and relay a query from an instruction to a database. In some embodiments, instructions can store or retrieve datasets from an elastic storage system, a local storage system, or both. In some embodiments, instructions comprise one or more states that indicate which instruction was last performed and/or which instruction is to be performed next. In some embodiments, instructions automatically logs events (e.g., errors or performance issues) that occur while the instructions are performed.

Containers for instructions can be deployed on serverless computing instance. A first subset of the instructions can be retrieved and used on a first instance. A second subset of the instructions can be retrieved and used on a second instance. The first subset of the instructions and the second subset of the instructions can be orchestrated to be performed together using the first instance and the second instance in parallel. The size of the first instance and the second instance can be based on the complexity of the first subset of instructions, the second subset of instructions, the amount of the dataset to be processed, or any combination thereof.

Datasets can be stored and retrieved from a variety of storage systems. In some embodiments, a storage system can be a relational database. In some embodiments, a storage system can be a non-relational database. In some embodiments, a storage system can be a distributed database. In some embodiments, a storage system can be an object-based database.

### Numbered Embodiments

The following list of numbered embodiments of the invention are to be considered as disclosing various features of the invention, which features can be considered to be specific to the particular embodiment under which they are discussed, or which are combinable with the various other features as listed in other embodiments. Thus, simply because a feature is discussed under one particular embodiment does not necessarily limit the use of that feature to that embodiment.

Embodiment 1. A method for determining a polyamino acid descriptor associated with a biological state, comprising: removing technical variation from a proteomic dataset to generate a refined proteomic dataset, the technical variation arising from a predetermined non-biological factor, by training a neural network to decrease a loss function configured to: increase a similarity between a first set of latent embeddings that are based on a first subset of polyamino acid descriptors in the proteomic dataset, wherein the first subset of polyamino acid descriptors are obtained from the same sample; and decrease the similarity between a second set of latent embeddings that are based on a second subset of polyamino acid descriptors in the proteomic dataset, wherein the second subset of polyamino acid descriptors are obtained from different samples; and identifying the polyamino acid descriptor that is associated with the biological state from the refined proteomic dataset.

Embodiment 2. The method of embodiment 1, wherein the proteomic dataset comprises a plurality of polyamino acid descriptors.

Embodiment 3. The method of embodiment 2, wherein the plurality of polyamino acid descriptors comprises a plurality of polyamino acid intensities.

Embodiment 4. The method of embodiment 3, wherein the plurality of polyamino acid intensities is based on a plurality of polyamino acid identifications, a plurality of surface types, or both.

Embodiment 5. The method of embodiment 4, wherein the polyamino acid descriptor associated with the biological state comprises a polyamino acid identification.

Embodiment 6. The method of embodiment 5, wherein the polyamino acid identification comprises a proteoform identification.

Embodiment 7. The method of any one of embodiments 1-6, wherein the similarity is quantified using a similarity function comprising a distance-based similarity function, an angle-based similarity function, a set-based similarity function, or any combination thereof.

Embodiment 8. The method of any one of embodiments 1-7, wherein a local inverse Simpson's index (LISI) score of the refined proteomic dataset is less than 1.1, 1.2, 1.3, 1.4, 1.5, 1.6, 1.7, 1.8, 1.9, or 2 for a biological factor.

Embodiment 9. The method of any one of embodiments 1-8, wherein a local inverse Simpson's index (LISI) score of the refined proteomic dataset is greater than 1, 1.1, 1.2, 1.3, 1.4, 1.5, 1.6, 1.7, 1.8, 1.9, or 2 for a biological factor.

Embodiment 10. The method of any one of embodiments 7-9, wherein the biological factor comprises a biological sample type, a surface type, or both.

Embodiment 11. The method of embodiment 10, wherein the surface type comprises a nanoparticle surface type.

Embodiment 12. The method of any one of embodiments 1-11, wherein a local inverse Simpson's index (LISI) score of the refined proteomic dataset is less than 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, or 4.0 for the predetermined non-biological factor.

Embodiment 13. The method of any one of embodiments 1-12, wherein a local inverse Simpson's index (LISI) score of the refined proteomic dataset is greater than 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, or 4.0 for the predetermined non-biological factor.

Embodiment 14. The method of any one of embodiments 1-13, wherein the predetermined non-biological factor comprises using a different machine, using a different chromatography column, measuring at a different location, measuring at a different time, measuring by a different user, or any combination thereof.

Embodiment 15. The method of any one of embodiments 1-14, further comprising receiving the plurality of polyamino acid descriptors measured from a plurality of mass spectrometers.

Embodiment 16. The method of any one of embodiments 1-15, further comprising receiving the plurality of polyamino acid descriptors measured at different locations.

Embodiment 17. The method of any one of embodiments 1-16, further comprising receiving the plurality of polyamino acid descriptors measured at different times.

Embodiment 18. The method of any one of embodiments 1-17, further comprising receiving the plurality of polyamino acid descriptors measured by different users.

Embodiment 19. The method of any one of embodiments 1-18, wherein the predetermined non-biological factor comprises collecting samples from a different location, collecting or processing samples by a different user, processing samples using different devices, transporting samples using a different condition, or any combination thereof.

Embodiment 20. The method of any one of embodiments 1-19, further comprising receiving the plurality of polyamino acid descriptors measured from samples collected from different locations.

Embodiment 21. The method of any one of embodiments 1-20, further comprising receiving the plurality of polyamino acid descriptors measured from samples collected or processed by different users.

Embodiment 22. The method of any one of embodiments 1-21, further comprising receiving the plurality of polyamino acid descriptors measured from samples processed using different devices.

Embodiment 23. The method of any one of embodiments 1-22, further comprising receiving the plurality of polyamino acid descriptors measured from samples transported under different conditions.

Embodiment 24. The method of any one of embodiments 15-23, wherein the receiving is through the cloud.

Embodiment 25. The method of embodiment 24, further comprising: obtaining a plurality of mass spectrometry datasets obtained from a plurality of samples; normalizing, using a plurality of computing nodes, across the plurality of mass spectrometry datasets to generate a plurality of normalized mass spectrometry datasets, wherein the proteomic dataset comprises the plurality of normalized mass spectrometry datasets.

Embodiment 26. The method of embodiment 25, wherein the normalizing generates a set of aligned precursors for each mass spectrometry dataset in the plurality of mass spectrometry datasets.

Embodiment 27. The method of embodiment 25 or 26, wherein the normalizing generates a set of relative abundances for each mass spectrometry dataset in the plurality of mass spectrometry datasets.

Embodiment 28. The method of any one of embodiments 25-27, wherein the normalizing comprises adjusting a set of polyamino acid intensities for each mass spectrometry dataset in the plurality of mass spectrometry datasets based on a plurality of feature values determined from the plurality of mass spectrometry datasets.

Embodiment 29. The method of any one of embodiments 25-28, wherein the normalizing comprises minimizing an objective function for a unique pair of mass spectrometry datasets in the plurality of mass spectrometry datasets for each computing node in the plurality of computing nodes.

Embodiment 30. The method of any one of embodiments 25-29, further comprising generating a harmonized plurality of mass spectrometry datasets comprising a harmonized format based on the plurality of mass spectrometry datasets, wherein the harmonized format comprises (i) the plurality of mass spectrometry datasets in an indexed series and (ii) indices of the indexed series, such that the harmonized format is capable of being read in arbitrary slices in the indexed series and of inserting new datasets and/or being modified between arbitrary indices in the indexed series;

Embodiment 31. The method of any one of embodiments 1-30, further comprising: generating, based at least in part on a genomic dataset, a set of expressible proteoforms that can be expressed from a set of nucleic acids in the genomic dataset; and mapping the refined proteomic dataset to the set of expressible proteoforms, thereby determining a set of expressed proteoforms in the biological sample, wherein the polyamino acid descriptor is a proteoform in the set of expressed proteoforms.

Embodiment 32. A method of correcting batch effects in proteomic data, comprising: providing a neural network comprising: an input layer configured to receive at least one polyamino acid descriptor; a latent layer configured to output at least a latent descriptor, wherein the latent layer is operably connected to the input layer; and an output layer operably connected to the latent layer; wherein the latent layer and the output layer comprises one or more parameters; providing training data comprising a plurality of the at least one polyamino acid descriptors, wherein the plurality of polyamino acid descriptors comprises at least one value for a measured intensity of a given polyamino acid; training the neural network, by (i) inputting at least the plurality of polyamino acid descriptors at the input layer of the neural network, (ii) outputting a plurality of latent descriptors at the latent layer and a plurality of outputs at the output layer, and (iii) optimizing a loss function that is configured to guide the neural network towards learning a latent space comprising a plurality of embeddings for the plurality of polyamino acid descriptors by updating the one or more parameters, wherein the plurality of embeddings is invariant with respect to a predetermined non-biological factor.

Embodiment 33. The method of embodiment 32, further comprising reconstructing, using a decoder neural network connected to the latent layer, a given plurality of polyamino acid descriptors based at least in part on a given plurality of embeddings to output a plurality of reconstructed polyamino acid descriptors, such that the plurality of reconstructed polyamino acid descriptors has a reduced variance with respect to the predetermined non-biological factor as compared to the plurality of polyamino acid descriptors.

Embodiment 34. The method of embodiment 32 or 33, wherein the predetermined non-biological factor comprises at least one of: location of measurement, time of measurement, instrumentation component, or any combination thereof.

Embodiment 35. The method of embodiment 34, wherein the instrumentation component comprises a mass spectrometry column.

Embodiment 36. The method of embodiment any one of embodiments 32-35, wherein the loss function comprises an adversarial triplet objective function comprising: L(a, p, n)=min Σᵢ₌₁^{N} max(d(aᵢ, p)−d(aᵢ, n)+α, 0), wherein a denotes a polyamino acid descriptor, wherein p denotes a positive reference for the polyamino acid descriptor, wherein n denotes a negative reference for the polyamino acid descriptor, and wherein a denotes a margin parameter.

Embodiment 37. The method of embodiment 36, wherein the loss function further comprises a classification loss function.

Embodiment 38. The method of embodiment 37, wherein the classification loss function is configured to classify between distinct biological samples, distinct assay methods, or both.

Embodiment 39. The method of embodiment 38, wherein the distinct assay methods comprises assays using distinct nanoparticles.

Embodiment 40. The method of any one of embodiments 32-36, wherein the loss function further comprises a reconstruction loss function.

Embodiment 41. The method of any one of embodiments 32-40, wherein the measured intensity comprises peptide intensity or protein group intensity.

Embodiment 42. The method of any one of embodiments 32-41, wherein the latent layer and the input layer are operably connected via one or more hidden layers.

Embodiment 43. The method of any one of embodiments 32-42, wherein the latent layer and the output layer are operably connected via one or more hidden layers.

Embodiment 44. A method of correcting batch effects in omic data, comprising: providing a neural network comprising: an input layer configured to receive at least one omic descriptor; a latent layer configured to output at least a latent descriptor, wherein the latent layer is operably connected to the input layer; and an output layer operably connected to the latent layer; wherein the latent layer and the output layer comprises one or more parameters; providing training data comprising a plurality of the at least one omic descriptors wherein the plurality of omic descriptors comprises at least one value for a measured intensity of a given omic signal; and training the neural network, by (i) inputting at least the plurality of omic descriptors at the input layer of the neural network, (ii) outputting a plurality of latent descriptors at the latent layer and a plurality of outputs at the output layer, and (iii) optimizing a loss function that is configured to guide the neural network towards learning a latent space comprising a plurality of embeddings for the plurality of omic descriptors by updating the one or more parameters, wherein the plurality of embeddings is invariant with respect to a predetermined non-biological factor.

Embodiment 45. A method for determining an omic descriptor associated with a biological state, comprising: removing technical variation from an omic dataset to generate a refined omic dataset, the technical variation arising from a predetermined non-biological factor, by training a neural network to decrease a loss function configured to: increase a similarity between a first set of latent embeddings that are based on a first subset of omic descriptors in the omic dataset, wherein the first subset of omic descriptors are obtained from the same sample; and decrease the similarity between a second set of latent embeddings that are based on a second subset of omic descriptors in the omic dataset, wherein the second subset of omic descriptors are obtained from different samples; and identifying the omic descriptor that is associated with the biological state from the refined omic dataset.

Embodiment 46. A computer-implemented method, implementing any one of the methods of embodiments 1-45 in a computer.

Embodiment 47. A computer program product comprising a computer-readable medium having computer-executable code encoded therein, the computer-executable code adapted to be executed to implement any one of the methods of embodiments 1-45.

Embodiment 48. A non-transitory computer-readable storage media encoded with a computer program including instructions executable by one or more processors to implement any one of the methods of embodiments 1-45.

Embodiment 49. A computer-implemented system comprising: a digital processing device comprising: at least one processor, an operating system configured to perform executable instructions, a memory, and a computer program including instructions executable by the digital processing device to implement any one of the methods of embodiments 1-45.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

### EXAMPLES

The following examples are provided to further illustrate some embodiments of the present disclosure, but are not intended to limit the scope of the disclosure; it will be understood by their exemplary nature that other procedures, methodologies, or techniques known to those skilled in the art may alternatively be used.

### Example 1: Cloud Scalable Omics Data Analysis Pipeline

A cloud scalable omics data analysis pipeline may begin with Watchdog monitors that can transfer MS files, as they arrive, from one or more LCMS instruments into AWS S3 file storage. The transfer may trigger Lambda Functions, which can act as a connection to one or more Step Functions, which can map out tasks, choices, and error-handling that may be used for the analysis of MS data. Elastic Container Service Tasks, which may execute computationally rigorous code, may use Docker-containerized executables that may be instantiated using a mixture of AWS's Fargate and Batch serverless paradigm. In some cases, Batch may be leveraged when Fargate's compute and local storage is not sufficient. In some cases, Batch with Spot Instances may be leveraged for short but intense jobs to reduce costs. In some cases, the cloud scalable omics data analysis pipeline outputs may be stored in a combination of S3 buckets, a non-relational Mongo database, and a relational PostgreSQL database, which may operate on a principle of polyglot persistence. In some cases, differently structured data may be stored in different types of databases. In some cases, highly structured experimental data may be stored in a relational PostgreSQL database (SeerDB). In some cases, instrument readings and quality control data may be stored in non-relational MongoDB database. In some cases, APIs and various internal applications may be used to query one or more datastores to return information collectively. In some cases, the cloud scalable omics data analysis pipeline may comprise massively parallel group run contexts.

Seer's current database contains at least about 500 terabytes of raw, semi-structured and structured data from a fleet of LCMS instruments from multiple vendors. Peptide and protein annotations are query-able using a polyglot persistence model of document and relational systems. In some cases, thousands of peptide and protein annotations are query-able using a polyglot persistence model of document and relational systems. Cloud-first laboratory pipes data using an Amazon Web Services (AWS) storage gateway may service and automatically process raw data using event based-triggering mechanisms. Users may also launch group analysis runs with pre-defined recipes. The described architecture may rely on open source algorithm components. In some cases, the cloud scalable omics data analysis pipeline may analyze thousands of samples in hours. The cloud scalable omics data analysis pipeline may support hundreds of terabytes of incoming LCMS data, annually. The cloud scalable omics data analysis pipeline may process at least about 150 files with 140 AWS Batch jobs per day. The cloud scalable omics data analysis pipeline may process at least about 2600 AWS Fargate tasks per day.

### Example 2: Large Scale, Cloud Enabled Re-Analysis

In an example, the Proteograph™ technology may be applied to cancer cohorts to identify protein groups across an entire cohort. Data was acquired in data-independent-acquisition (DIA) mode on a Sciex Triple TOF 6600+ with EKSPERT nano-LC 425 LC running a 33 min gradient. Previously, computational resources limited large-scale group analysis of the data, but using new scalable cloud infrastructure enabled processing of the entire cohort in one large group-run using DIA-NN v1.8 in library free mode using the --relaxed-prot-inf flag. Downstream analysis, including variational autoencoder (VAE) neural network, may be built on top of open-source python libraries.

Large-scale re-analysis yielded nearly 4,000 protein groups across the entire cohort with each sample averaging over 2,000 protein groups. This corresponds to about a 5-fold increase in depth compared to neat plasma, which is typically around 400 protein groups per sample. This corresponded to nearly 25% increase per sample from a prior analysis. The increased depth may be due to a combination of more sensitive library-free search and a large group run combining all the injections. Injections may be combined after acquisition (e.g., MS acquired spectrums). Cloud-based architecture may enable protein grouping through combining multiple injections to create the most comparable group.

### Example 3: Optimizing LCMS Algorithms to Leverage Distributed Computing Engines

False Discovery Rate (FDR) controlled Protein identification results can include several steps. First, Protein Spectrum Matches (PSMs) and uniquely identified peptides may be generated from each individual injection. This step may be rather flexible as they may be run as an individual file, multiple files on the same machine or different files ran on different machines in parallel (e.g. Fargate). Bottlenecks may appear when data aggregation steps are are used where files are aggregated before processing. For example, the MSFragger search engine component of Fragpipe may process two thousand files in a few hours using autoscaling features of AWS batch or Fargate, however, Protein Prophet adds significant overhead (e.g., days) to the processing time to process even on a large instance. Using the distributed compute engine Apache Spark may relieve a bottleneck. These components may seamlessly interact, and more complex and scalable pipelines may be created

The most critical bottleneck in a group run workflow may be the protein inference step, where results from all runs are pooled and analyzed simultaneously, straining both memory and compute. In some cases, this is the only step that may scale linearly with respect to number of runs. For example, in an MsFragger group run of over 2300 injections, this step, conducted by ProteinProphet, takes over 30 hours, which may be far more than half of the total runtime.

In some cases, one approach, used in MaxQuant, Alphapept, and other engines, aims to solve a protein inference problem using a protein and peptide graph network and a razor approach (Tyanova et al, 2016). After creating a network with connections between all peptides and proteins, the proteins with the most peptide connections may be iteratively selected as the “razor protein” and removed from the graph. This approach may be a simpler solution than PeptideProphet's approach, which may enable a design for a distributed approach that will ease the computational bottleneck. Apache Spark may enable scaling efficiently.

### Example 4: Domain Adversarial Neural Networks for Learning Batch-Invariant Representations

This example describes an adversarial neural network architecture for learning batch invariant representations. Domain adversarial neural networks (DANNs) are adapted for learning batch-invariant representations with some modifications: 1. A multi-task classification objective for the main learning task or an unsupervised reconstruction loss; 2. A triplet loss rather than a classification loss for the adversarial task.

The architecture (termed DannClf, shown in FIG. 16) comprises an encoder (h), two classification heads (f and g), and an adversarial triplet loss computed on h, after the embeddings go through a gradient reversal layer (GRL). The input to the network is the log₁₀ intensity of the protein groups, and all layers except the classifier outputs use ReLU activations. For layers in h, before each non-linear activation, dropout (p=0.1) and batch-normalization are applied. After h, dropout (p=0.1) is applied before the classifier layers. For a given input triplet (anchor sample, positive sample, and negative sample), the loss off and g are computed on the anchor, and the full triplet is used for the adversarial triplet loss. The network is trained using the Adam optimizer with a learning rate of 1e⁻³, and the best checkpoint over 1000 epochs is kept based on the validation loss of f.

For the reconstruction-loss variant (DannRecon), the same encoder is used as in DannClf, but with a decoder (d) that mirrors this architecture and uses tied weights (weight sharing) with the encoder. The same optimizer settings are used, and the model is trained over 3000 epochs, keeping the best checkpoint based on validation mean-squared error (MSE) loss.

Conditional triplet mining for multiple tasks: The adversarial component of the original DANN model was tasked with discriminating between samples that come from the source or target domains via a negative log-likelihood loss. However, to prioritize learning good representations of the data and not just classification, a metric learning loss is used. Some issues with Siamese or triplet approaches that don't consider multi-task labels for domain adaptation are: 1. Pair mining is unconstrained and leads to a quadratic growth (or cubic growth for triplets) of the training set; meanwhile randomly picking amongst these many pairs is unlikely to pick “informative” pairs that result in non-zero loss, and learning may be slow; 2. When labels between the source and target domains do not fully overlap, the learned features are not guaranteed to preserve original label structure (i.e. samples from the same label may be pulled apart and samples from different labels might be pushed together in the target domain). This can be problematic in biomedical settings, where inspection of learned features that lead to classification decisions is important for interpretation.

To address this, a conditional mining strategy is adopted for triplets and accounting for multiple tasks. Triplets that are selected are strategically constrained for training using labels from both tasks, so that for a given anchor sample, it is selected: A positive sample which comes from the same batch (Machine), but is a different Biosample AND different NP compared to the anchor; A negative sample which comes from a different batch (Machine), but is the same Biosample AND same NP compared to the anchor.

### Example 5: Characterizing and Correcting Batch Effects in Large Scale Proteomics

Advances in LSMS-based proteomics analysis have enabled the efficient profiling of thousands of proteins from single LCMS runs. The ability to run untargeted, high throughput LCMS experiments has opened the door to large-scale cohort studies for biomarker and drug target discovery. When conducting large-scale cohort studies, technical confounding can be introduced as samples are run across different MS instruments, LC columns, dates, and geographic locations. In order to integrate these samples across datasets for joint analyses, one may diagnose such batch effects and apply methods to correct them.

Batch correction is an important problem in the biomedical field. Some batch correction methods used in proteomics, transcriptomics, and other omics are nonparametric to reduce assumptions made on the data. Some examples are methods based on simple median normalization as in MSStats; nearest neighbor matching like MNN and Scanorama; and Harmony which is aniterative clustering and vector translating algorithm. Parametric approaches include ComBat which is based on empirical Bayes, and deep-learning based approaches such as scVI.

The field of domain adaptation in machine learning is closely related to this problem, where models trained under a source domain are tasked with predicting on a target domain, and data in each case may come from different underlying distributions (domain shift).

This example illustrates a method for characterizing and/or correcting batch effects in proteomics data. Supervised adversarial neural network is trained to learn batch-invariant representations of proteomics data. The neural network is benchmarked against other batch correction methods, for example, the presence of batch effects are characterized using several methods, including Principal Components Analysis-based approaches, local-neighborhood diversity measures, and machine learning classifier-based methods. The neural network shows ability to remove technical variation, leading to about 20% improvement in dataset homogenization while preserving biological variation better than other methods.

Batch-diverse dataset was created, which includes 882 LCMS (DDA) runs across: two types of control plasma samples, three Seer Proteograph nanoparticles, three LCMS instruments, and eight LC columns.

Various batch effect correction methods (including PCA, MSStats, ComBat, MNN, Scanorama, Harmony, scVI, and domain adversarial neural network (DANN)) were applied to the dataset to create batch-corrected representations. The batch-corrected representations were evaluated with various metrics as described herein.

Principle component analysis (PCA) regression metric: PCA can be used for (i) de-noising by only considering top principle components (PCs), dimensionality reduction, and visualization. Scatter plots of data in the first few PCs can be used as a quick qualitative check for discriminative signals, including those based on biological variables as well as technical covariates (batch effects). Assessing magnitude of signal in variables relative to each other can be difficult with qualitative approaches such as PCA due to differences in data density and variance residing in PCs other than PC1 and PC2. To address this, a quantitative score called principal component regression is used, which is based on PCA of a data matrix in conjunction with simple linear regression with covariates.

PCT Reg. Score=Var(X|B)≈Σᵢ₌₁^{G} Var(X|PCᵢ)·R²(PCᵢ|B),
where B is the batch variable (e.g. MS Instrument), X is the protein group log-intensity data matrix, and PCᵢ is the i-th principal component. The notation Var(A|B) denotes the variance of A explained by B. R²(PCᵢ|B) is the coefficient of determination from simple linear regression of PCᵢ˜B. In our study, we set G=50, and only allow PCs to contribute to the sum if their linear regression fit is significant (<0.05 p-value of the F-statistic). Intuitively, the PCA regression score reveals where the signal resides in the protein group intensity matrix.

Local Inverse Simpson's Index (LISI) score: LISI score is used determine how well datasets are integrated in a common space. LISI approximates the effective diversity of a label within small neighborhoods of the data. It is computed around each point (LCMS run) and its distribution across all points can be inspected.

$$LISI Score = \frac{1}{{\sum }_{i = 1}^{K} {p}_{i}^{2}}$$

Where K is the number of categories (types) for the variable of interest, and pᵢ is the probability of category i in the neighborhood.

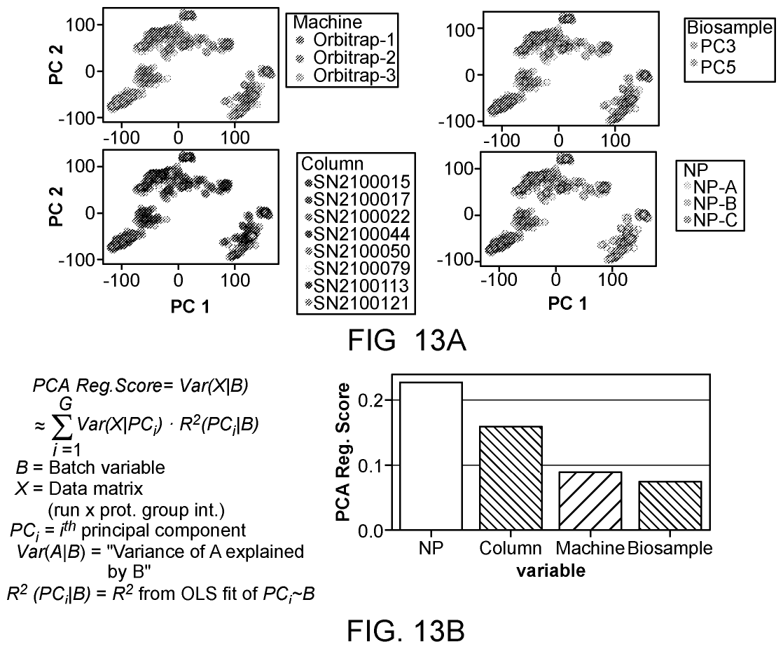

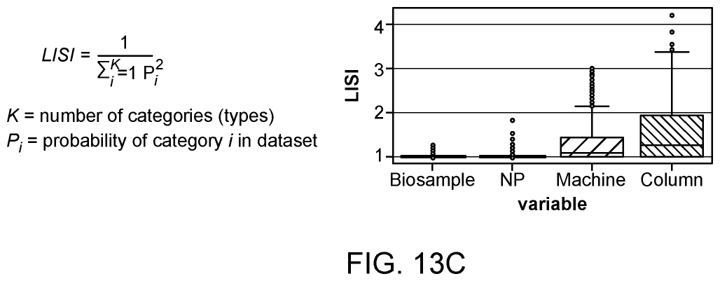

FIG. 13A shows PCA embeddings of protein group log intensities of each run for four different covariates. FIG. 13B shows principal components regression, showing that batch variables (LC column and MS instruments) add significant variance to the data over the analysis of the control plasma samples. FIG. 13C shows Local Inverse Simpson's Index (LISI) score, which measures effective diversity of a label within small neighborhoods, which shows low levels of integration of batch variables. FIG. 13B shows where signal resides in a data matrix and FIG. 13C shows the level of mixing.

Dataset: A batch-diverse dataset was collected using Seer's Proteograph™ Product Suite. The dataset includes data from 882 LC-MS runs (using a 30 minute gradient length with data dependent acquisition (DDA)) across: two types of pooled plasma samples (PS), three Seer Proteograph nanoparticles (NPs), three LCMS instruments (aka machines), and eight LC columns. Data was processed with MaxQuant/Andromeda (v1.6.10.43).

Characterizing technical effects in the data: The data matrix was projected (rows: LC-MS runs, columns: protein groups, values:log₁₀ intensities) onto the first two principal components, and observed that there was separation by a technical variable (Machine) within clusters of biologically-relevant variables (Biosample and Nanoparticle). In particular, separation was observed in samples that are PS1 and NP3, as they separate by whether they were run on Orbitrap-1 or Orbitrap-3 (FIG. 13A). These technical effects are quantitatively assessed by computing PCA regression scores for these variables. It was seen that Column and Machine (technical variables) can both explain more of the variance in the data than Biosample (FIG. 13B).

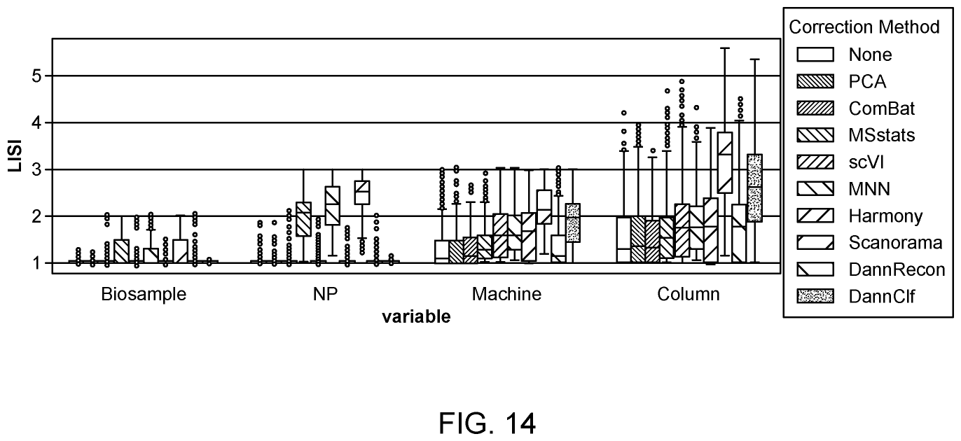

Data integration performance comparison: Various methods including the DannClf and DannRecon methods were evaluated in the dataset to produce batch-corrected representations, which was then evaluated with the LISI metric. FIG. 14 shows dataset mixing and biological signal preservation of batch effect correction methods. LISI scores of correction methods are shown. Though Scanorama mixed the best with respect to variance in Machine and Column, it overmixed the biological variables. The DannClf and DannRecon did not exhibit the same issue.

An optimal method would return a representation which exhibits minimal mixing with respect to biological variables (preserving biological signal, so lower LISI scores are better), while simultaneously exhibiting high mixing with respect to technical variables (data integration, higher LISI scores are better). We observe that commonly used approaches such as median normalization (MSstats) and MNN do not preserve the biological signal as well as other methods, as their median LISI scores are well above 1 for Biosample and NP. On the other hand, we see that Scanorama does very well in data integration across technical variables, achieving the highest median LISI score on Machine and Column. However, this may come at the cost of over-mixing, as its LISI score on biological variables is well above 1. On the otherhand, we see that both our DannClf and DannRecon models are able to maintain biological signal by keeping LISI at its lowest possible value of 1.0 for both Biosample and NP. At the same time, DannClf achieves the second highest median LISI score on the technical variables (second to Scanorama, which may have over-mixed the data).

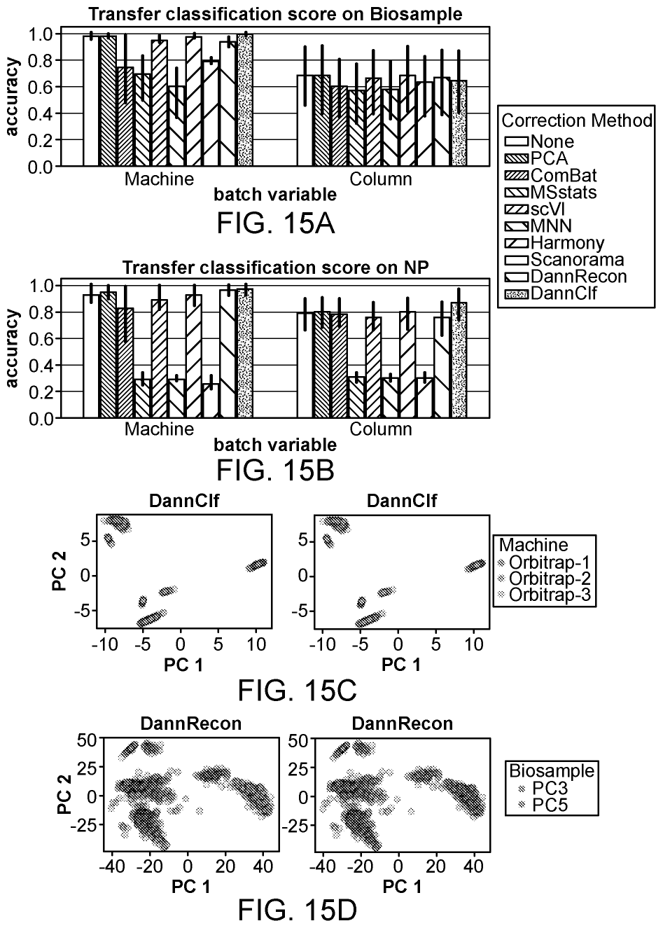

Classification using batch-corrected representations: The panel of batch correction methods are assessed based on how well their learned representations can be used for downstream tasks. In particular, the utility of these representations for classification across technical batches is assessed (whether or not they can be used for transfer learning). Since the dataset has more nanoparticles than biosamples, and is more balanced in this variable, it is used as the prediction task. Each batch correction method is applied the dataset, then independent k-nearest neighbors (KNN) classifier was used to predict the nanoparticle. However, the training set for the KNN model has samples from two mass spectrometry machines, with samples from the third mass spectrometry machine completely held out. Test accuracy is computed on the held-out data. The process is repeated two more times, holding out each of the other two machines, and attain the average test accuracy. This process is also repeated for holding out on the Column variable. FIG. 15A shows comparison of using batch corrected representations for classifying biological phenotypes across batches, which indicates that the nanoparticle signature is strong (high accuracy without batch correction), but is boosted by our DannClf and DannRecon models, while accuracy using other methods is reduced. FIG. 15B shows testing results on a prediction task for classifying between the three nanoparticles. FIG. 15C shows PCA embeddings of the learned features from the DannClf and DannRecon models.

Conclusion: Batch effects can contribute to a large amount of the noise in large-scale proteomics datasets compared to biological variations. A batched LC-MS plasma proteomics dataset was collected, and using qualitative and quantitative (PCA regression scoring), a batch effect attributed to mass spectrometers and LC columns were observed in the dataset. An extension of DANNs was introduced based on multi-task learning and the triplet adversarial loss, and a conditional triplet mining strategy was used to efficiently train it. The method was benchmarked alongside other batch-effect correction methods. The DannClf model showed the ability harmonize data across technical factors, while maintaining the fidelity of the biological signal in the data. While DannClf can harmonize the data well, the representations it learns are may be useful for classification. The unsupervised variant, DannRecon, may learn more general-purpose batch corrected representations.

Batch effects can contribute to a significant amount of noise in large-scale proteomic datasets, relative to variance from biological factors. A significant batch effect is observed, which can be attributed mass spectrometers and LC columns. Deep learning-based approaches can be used to integrate diverse proteomics datasets. The implementations of DANN (DannClf and DannRecon) can harmonize data across technical factors, while maintaining the fidelity of the biological signal in the data. DannClf shows the ability to learn representations that are useful for classification. DannRecon may learn more general-purpose batch corrected representations.

1. A method for determining a polyamino acid descriptor associated with a biological state, comprising:
(a) removing technical variation from a proteomic dataset to generate a refined proteomic dataset, the technical variation arising from a predetermined non-biological factor, by training a neural network to decrease a loss function configured to:
i. increase a similarity between a first set of latent embeddings that are based on a first subset of polyamino acid descriptors in the proteomic dataset, wherein the first subset of polyamino acid descriptors are obtained from the same sample; and
ii. decrease the similarity between a second set of latent embeddings that are based on a second subset of polyamino acid descriptors in the proteomic dataset, wherein the second subset of polyamino acid descriptors are obtained from different samples; and
(b) identifying the polyamino acid descriptor that is associated with the biological state from the refined proteomic dataset.

2. The method of claim 1, wherein the proteomic dataset comprises a plurality of polyamino acid descriptors.

3. The method of claim 2, wherein the plurality of polyamino acid descriptors comprises a plurality of polyamino acid intensities.

4. The method of claim 3, wherein the plurality of polyamino acid intensities is based on a plurality of polyamino acid identifications, a plurality of surface types, or both.

5. The method of claim 4, wherein the polyamino acid descriptor associated with the biological state comprises a polyamino acid identification.

6. The method of claim 5, wherein the polyamino acid identification comprises a proteoform identification.

7. The method of claim 1, wherein the similarity is quantified using a similarity function comprising a distance-based similarity function, an angle-based similarity function, a set-based similarity function, or any combination thereof.

8. The method of claim 1, wherein a local inverse Simpson's index (LISI) score of the refined proteomic dataset is greater than 1, 1.1, 1.2, 1.3, 1.4, 1.5, 1.6, 1.7, 1.8, 1.9, or 2 for a biological factor.

9. The method of claim 8, wherein the biological factor comprises a biological sample type, a surface type, or both.

10. (canceled)

11. The method of claim 1, wherein a local inverse Simpson's index (LISI) score of the refined proteomic dataset is less than 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, or 4.0 for the predetermined non-biological factor.

12. The method of claim 1, wherein the predetermined non-biological factor comprises collecting samples from a different location, collecting or processing samples by a different user, processing samples using different devices, transporting samples using a different condition, or any combination thereof.

13. The method of claim 1, further comprising receiving the plurality of polyamino acid descriptors measured from samples collected or processed by different users.

14. The method of claim 1, wherein the predetermined non-biological factor comprises using a different machine, using a different chromatography column, measuring at a different location, measuring at a different time, measuring by a different user, or any combination thereof.

15. The method of claim 1, further comprising receiving the plurality of polyamino acid descriptors measured at different locations.

16. (canceled)

17. The method of claim 15, further comprising:
(a) obtaining a plurality of mass spectrometry datasets obtained from a plurality of samples; and
(b) normalizing, using a plurality of computing nodes, across the plurality of mass spectrometry datasets to generate a plurality of normalized mass spectrometry datasets, wherein the proteomic dataset comprises the plurality of normalized mass spectrometry datasets.

18. The method of claim 17, wherein the normalizing generates a set of aligned precursors for each mass spectrometry dataset in the plurality of mass spectrometry datasets.

19. The method of claim 17, wherein the normalizing generates a set of relative abundances for each mass spectrometry dataset in the plurality of mass spectrometry datasets.

20. The method of claim 1, further comprising:
(a) generating, based at least in part on a genomic dataset, a set of expressible proteoforms that can be expressed from a set of nucleic acids in the genomic dataset; and
(b) mapping the refined proteomic dataset to the set of expressible proteoforms, thereby determining a set of expressed proteoforms in the biological sample, wherein the polyamino acid descriptor is a proteoform in the set of expressed proteoforms.

21. A method of correcting batch effects in proteomic data, comprising:
(a) providing a neural network comprising:
i. an input layer configured to receive at least one polyamino acid descriptor;
ii. a latent layer configured to output at least a latent descriptor, wherein the latent layer is operably connected to the input layer; and
iii. an output layer operably connected to the latent layer;
wherein the latent layer and the output layer comprises one or more parameters;
(b) providing training data comprising a plurality of the at least one polyamino acid descriptors, wherein the plurality of polyamino acid descriptors comprises at least one value for a measured intensity of a given polyamino acid;
(c) training the neural network, by (i) inputting at least the plurality of polyamino acid descriptors at the input layer of the neural network, (ii) outputting a plurality of latent descriptors at the latent layer and a plurality of outputs at the output layer, and (iii) optimizing a loss function that is configured to guide the neural network towards learning a latent space comprising a plurality of embeddings for the plurality of polyamino acid descriptors by updating the one or more parameters, wherein the plurality of embeddings is invariant with respect to a predetermined non-biological factor.

22. A method of correcting batch effects in omic data, comprising:
(a) providing a neural network comprising:
i. an input layer configured to receive at least one omic descriptor;
ii. a latent layer configured to output at least a latent descriptor, wherein the latent layer is operably connected to the input layer; and
iii. an output layer operably connected to the latent layer;
wherein the latent layer and the output layer comprises one or more parameters;
(b) providing training data comprising a plurality of the at least one omic descriptors wherein the plurality of omic descriptors comprises at least one value for a measured intensity of a given omic signal; and
(c) training the neural network, by (i) inputting at least the plurality of omic descriptors at the input layer of the neural network, (ii) outputting a plurality of latent descriptors at the latent layer and a plurality of outputs at the output layer, and (iii) optimizing a loss function that is configured to guide the neural network towards learning a latent space comprising a plurality of embeddings for the plurality of omic descriptors by updating the one or more parameters, wherein the plurality of embeddings is invariant with respect to a predetermined non-biological factor.

23. (canceled)
